(19) United States
(12) Patent Application Publication
(10) Pub. No.: US 2026/0023089 A1
(43) Pub. Date: Jan. 22, 2026

(54) GENETICALLY ENCODED BIOLUMINESCENT SENSORS

(71) Applicant: Central Michigan University, Mount Pleasant, MI (US)

(72) Inventor: Eric PETERSEN, Mount Pleasant, MI (US)

(21) Appl. No.: 19/145,138

(22) PCT Filed: Jan. 5, 2024
(86) PCT No.: PCT/US24/10425

§ 371 Date: Jul. 1, 2025

Related U.S. Application Data
(60) Provisional application No. 63/478,805, filed on Jan. 6, 2023.

(51) Int. Cl. G01N 33/94; C07K 14/705; C12N 9/02; C12N 15/62; C12N 15/86; C12Q 1/66; G01N 21/76; G01N 33/66

(52) U.S. Cl. CPC G01N 33/9426; C07K 14/70571; C12N 9/0069; C12N 15/62; C12N 15/86; C12Q 1/66; G01N 21/763; G01N 33/66; G01N 33/9413; G01N 33/942; G01N 33/944; C07K 2319/61; C12N 2750/14143; C12Y 113/12005; G01N 2333/70571; G01N 2333/90241

(57) ABSTRACT

Described herein are compositions and methods for bioluminescent analyte detection. In some embodiments, a recombinant bioluminescent polypeptide sensor is disclosed comprising a luminescent signaling domain, an analyte binding domain, and one or more peptide linkers, wherein the luminescent signaling domain is oriented in relation to the analyte binding domain such that binding of an analyte to the analyte binding domain induces a conformational change in the luminescent signaling domain to generate a luminescent signal.

### CROSS-REFERENCE TO RELATED APPLICATIONS

This application claims priority to U.S. Provisional Patent Application No. 63/478,805, filed on Jan. 6, 2023, which is incorporated by reference herein in its entirety.

### REFERENCE TO SEQUENCE LISTING

This application was filed with a Sequence Listing XML in ST.26 XML format accordance with 37 C.F.R. § 1.831. The Sequence Listing XML file submitted in the USPTO Patent Center, “026667-0003-WO01_sequence_listing_XML_26-DEC-2023.xml,” was created on Dec. 26, 2023, contains 26 sequences, has a file size of 89.1 Kbytes, and is incorporated by reference in its entirety into the specification.

### FIELD OF THE DISCLOSURE

This disclosure generally relates to bioluminescent analyte sensors.

### BACKGROUND

Optical biosensors have proven incredibly useful to researchers in a wide variety of fields for the study of neuronal and cellular activity. Genetically encoded optical sensors and advancements in microscopy instrumentation and techniques have revolutionized the scientific toolbox available for probing complex biological processes such as the release of specific neurotransmitters. These probes generate changes in light emission intensity and/or wavelength in response to physiological events such as calcium influx, membrane voltage changes, or the presence of a ligand such as a neurotransmitter.

Currently, nearly all available optical biosensors rely on fluorescence, which have been highly successful tools for single-cell imaging in superficial brain regions. However, there is much room for improvement and further development of new tools and approaches to better report neuronal changes in cellular dynamics in vivo within deeper brain structures without the need for hardware such as lenses or fibers that require implantation within the brain. One major improvement to such indicators would be to eliminate the need for an excitation light source, which is necessary to excite fluorescent reporters. This could be accomplished by replacing the fluorescent elements of existing biosensors with bioluminescent elements, which then eliminates the need of external light sources to illuminate the sensor and overcomes several drawbacks of fluorescence imaging such as limited light penetration depth, excitation scattering, and tissue heating, which are all associated with the external light needed for fluorescence imaging. Specifically, the illumination source used for fluorescent imaging is a major limiting factor for the depth at which cells can be imaged through tissue. This is due to the scattering of light traveling into the tissue, as well as heating as the incident photons are absorbed by endogenous molecules in the tissue.

Bioluminescence is produced by an enzyme (luciferase) that catalyzes oxidation of its specific substrate (i.e., a luciferin), resulting in the emission of light. Different forms of biological light production exist in multiple domains in nature, including beetles, worms, bacteria, and many marine organisms. Bioluminescence has been used for a variety of imaging applications, such as the quantification of gene expression over time and for imaging calcium dynamics to represent neuronal function or to track other calcium events within cells. Although bioluminescence has been used in research for decades, only recently have scientists been improving luciferases and synthetic luciferins. For example, a 1000-fold increase in luminescence was recently achieved by evolving firefly luciferase to use a synthetic substrate to create AkaLuc, which produces near infrared bioluminescence.

Imaging with bioluminescent probes eliminates the need for excitation light, circumventing the issues associated with fluorescence and enabling researchers to image deeper structures of the brain. Furthermore, autofluorescence produced by the illumination sources used for fluorescent imaging is not present when using bioluminescence, allowing for enhanced signal detection in deeper structures since the signal to noise ratio can be higher. These fundamental limitations to fluorescence imaging and corresponding benefits of bioluminescence imaging dovetail with an urgent need in the field of neuroscience for tools that allow recording and modulation of entire neuronal populations that are both non-invasive and do not require implanted hardware. These attributes also enable higher signal to noise ratios for in vitro assays such as recording cellular responses in drug screening assays.

Thus, what is needed are new and improved bioluminescent sensors and methods for detecting and reporting neuronal activity in vivo. These bioluminescent sensors and methods would be useful in various research and clinical applications for in vivo detection of analytes such as neurotransmitters, ultimately allowing neuroscientists to monitor activity associated with a specific neurotransmitter as it relates to behavior in a variety of neuronal and psychiatric disorders, among many other applications.

### SUMMARY

One embodiment described herein is a recombinant bioluminescent polypeptide sensor comprising: (a) a luminescent signaling domain; (b) an analyte binding domain; and (c) one or more peptide linkers; wherein the luminescent signaling domain is oriented in relation to the analyte binding domain such that binding of an analyte to the analyte binding domain induces a conformational change in the luminescent signaling domain to generate a luminescent signal. In one aspect, the sensor may further comprise (d) one or more cellular trafficking peptides such as membrane trafficking peptides. In another aspect, the luminescent signaling domain is allosterically regulated by the analyte binding domain such that signaling from the luminescent signaling domain is altered upon interaction of the analyte binding domain with the analyte. In another aspect, signaling by the luminescent signaling domain is proportional to the level of interaction between the analyte binding domain and the analyte. In another aspect, the luminescent signaling domain comprises a luciferase polypeptide. In another aspect, the luciferase polypeptide is split into two luciferase polypeptide domains, and wherein the analyte binding domain is present between the two luciferase polypeptide domains. In another aspect, the luciferase polypeptide emits luminescence at a wavelength ranging from about 450 nm to about 540 nm. In another aspect, the analyte binding domain comprises a neurotransmitter binding domain. In another aspect, the neurotransmitter binding domain comprises one or more glutamate binding domains, or functional variants, mutants, or fragments thereof. In another aspect, the neurotransmitter binding domain binds specifically to the neurotransmitter glutamate. In another aspect, the sensor is encoded by a polynucleotide sequence having at least 90-99% identity to any one of SEQ ID NO: 1, 3, 5, or 7. In another aspect, the sensor is encoded by a polynucleotide sequence selected from any one of SEQ ID NO: 1, 3, 5, or 7. In another aspect, the sensor has an amino acid sequence having at least 90-99% identity to any one of SEQ ID NO: 2, 4, 6, or 8. In another aspect, the sensor has an amino acid sequence selected from any one of SEQ ID NO: 2, 4, 6, or 8. In another aspect, the neurotransmitter binding domain comprises one or more dopamine binding domains, or functional variants, mutants, or fragments thereof. In another aspect, the neurotransmitter binding domain binds specifically to the neurotransmitter dopamine. In another aspect, the sensor is encoded by a polynucleotide sequence having at least 90-99% identity to SEQ ID NO: 9. In another aspect, the sensor is encoded by a polynucleotide sequence selected from SEQ ID NO: 9. In another aspect, the sensor has an amino acid sequence having at least 90-99% identity to SEQ ID NO: 10. In another aspect, the sensor has an amino acid sequence selected from SEQ ID NO: 10. In another aspect, the neurotransmitter binding domain comprises one or more acetylcholine binding domains, or functional variants, mutants, or fragments thereof. In another aspect, the neurotransmitter binding domain binds specifically to the neurotransmitter acetylcholine. In another aspect, the sensor is encoded by a polynucleotide sequence having at least 90-99% identity to any one of SEQ ID NO: 11, 13, or 15. In another aspect, the sensor is encoded by a polynucleotide sequence selected from any one of SEQ ID NO: 11, 13, or 15. In another aspect, the sensor has an amino acid sequence having at least 90-99% identity to any one of SEQ ID NO: 12, 14, or 16. In another aspect, the sensor has an amino acid sequence selected from any one of SEQ ID NO: 12, 14, or 16. In another aspect, the neurotransmitter binding domain comprises one or more GABA binding domains, or functional variants, mutants, or fragments thereof. In another aspect, the neurotransmitter binding domain binds specifically to the neurotransmitter GABA. In another aspect, the sensor is encoded by a polynucleotide sequence having at least 90-99% identity to any one of SEQ ID NO: 17, 19, or 21. In another aspect, the sensor is encoded by a polynucleotide sequence selected from any one of SEQ ID NO: 17, 19, or 21. In another aspect, the sensor has an amino acid sequence having at least 90-99% identity to any one of SEQ ID NO: 18, 20, or 22. In another aspect, the sensor has an amino acid sequence selected from any one of SEQ ID NO: 18, 20, or 22. In another aspect, the neurotransmitter binding domain comprises one or more serotonin binding domains, or functional variants, mutants, or fragments thereof. In another aspect, the neurotransmitter binding domain binds specifically to the neurotransmitter serotonin. In another aspect, the sensor is encoded by a polynucleotide sequence having at least 90-99% identity to SEQ ID NO: 23. In another aspect, the sensor is encoded by a polynucleotide sequence selected from SEQ ID NO: 23. In another aspect, the sensor has an amino acid sequence having at least 90-99% identity to SEQ ID NO: 24. In another aspect, the sensor has an amino acid sequence selected from SEQ ID NO: 24. In another aspect, the analyte binding domain comprises one or more glucose binding domains, or functional variants, mutants, or fragments thereof. In another aspect, the analyte binding domain binds specifically to glucose. In another aspect, the sensor is encoded by a polynucleotide sequence having at least 90-99% identity to SEQ ID NO: 25. In another aspect, the sensor is encoded by a polynucleotide sequence selected from SEQ ID NO: 25. In another aspect, the sensor has an amino acid sequence having at least 90-99% identity to SEQ ID NO: 26. In another aspect, the sensor has an amino acid sequence selected from SEQ ID NO: 26.

Another embodiment described herein is a vector comprising a polynucleotide sequence encoding any one of the recombinant bioluminescent polypeptide sensors disclosed herein. In one aspect, the vector is selected from a viral vector, a plasmid expression vector, an adeno-associated virus (AAV) vector, a recombinant AAV (rAAV) vector, a single-stranded AAV vector, a double-stranded AAV vector, or a self-complementary AAV (scAAV) vector. In another aspect, the vector is an AAV vector of a serotype selected from AAV1, AAV2, AAV3, AAV4, AAV5, AAV6, AAV7, AAV8, AAV9, AAV10, AAV11, AAV12, or a hybrid serotype thereof. In another aspect, the vector is a pcDNA3.1 plasmid expression vector.

Another embodiment described herein is a cell comprising any one of the vectors disclosed herein. In one aspect, the cell is a mammalian cell from a human, mouse, rat, dog, cat, pig, goat, rabbit, cow, horse, or other non-human primate.

Another embodiment described herein is a method for detecting one or more analytes in a subject, the method comprising measuring a level of luminescence emitted by any one of the recombinant bioluminescent polypeptide sensors disclosed herein and correlating the measured level of luminescence with the presence of the one or more analytes in the subject. In one aspect, the recombinant bioluminescent polypeptide sensor is encoded and expressed from a polynucleotide sequence that is administered to the subject. In another aspect, the polynucleotide sequence has at least 90-99% identity to any one of SEQ ID NO: 1, 3, 5, 7, 9, 11, 13, 15, 17, 19, 21, 23, or 25. In another aspect, the polynucleotide sequence is selected from any one of SEQ ID NO: 1, 3, 5, 7, 9, 11, 13, 15, 17, 19, 21, 23, or 25. In another aspect, the recombinant bioluminescent polypeptide sensor has an amino acid sequence having at least 90-99% identity to any one of SEQ ID NO: 2, 4, 6, 8, 10, 12, 14, 16, 18, 20, 22, 24, or 26. In another aspect, the recombinant bioluminescent polypeptide sensor has an amino acid sequence selected from any one of SEQ ID NO: 2, 4, 6, 8, 10, 12, 14, 16, 18, 20, 22, 24, or 26. In another aspect, the subject is a human. In another aspect, the subject is an animal. In another aspect, the subject is a non-human mammal.

Another embodiment described herein is the use of any one of the recombinant bioluminescent polypeptide sensors disclosed herein to detect one or more analytes in a subject.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A shows a schematic of a bioluminescent indicator/sensor for various analytes including neurotransmitters (e.g., acetylcholine, GABA, glutamate, dopamine, serotonin, norepinephrine) and other analytes (e.g., glucose) using split luciferase domains and an analyte sensing/binding domain displayed on the cell surface. FIG. 1B shows a comparison of bioluminescence imaging (right) and fluorescence imaging (left) for superficial and deep tissue targets in a rodent brain. Fluorescence imaging requires an excitation light which scatters as it interacts with the tissue, decreasing the ability to efficiently excite indicators as depth is increased. In comparison, bioluminescence is produced within the tissue through an enzymatic reaction of a luciferase (Luc) with its substrate (e.g., coelenterazine (CTZ) or furimazine, etc.) to generate light and an oxidized by-product (e.g., coelenteramide (CTD) or furimamide, etc.), which allows for increased imaging depth as an excitation light source is not the limiting factor.

FIG. 2A shows protein maps of three initial Bioluminescent Indicator of the Neurotransmitter Glutamate (“BLING”) designs that were tested. Shown from top to bottom are as follows: BLING 0.1-slow burn (sb) Gaussia luciferase split at 105-106 including the native Gaussia secretion peptide on the N terminal instead of the IGK leader secretion sequence; BLING 0.2-Nanoluc split at 66-67; and BLING 0.3-Nanoluc split at 159-160 (all three constructs with the Glt1 glutamate binding protein and the PDGFRβ transmembrane domain to anchor the sensor to the extracellular side of the membrane). FIG. 2B shows a protein map of BLING 1.0 with variable linkers. FIG. 2C shows results from the initial BLING constructs in response to 1 mM glutamate (n=4). FIG. 2D shows responses from the BLING linker library of ˜400 variants of BLING 0.2 with variable linkers that were tested.

FIG. 3A shows example luminescent responses of BLING 1.0 to 1 mM glutamate addition recorded with a plate reader. FIG. 3B shows responses of the parental construct (BLING 0.2) compared to optimized BLING (BLING 1.0) and the fluorescent glutamate indicator iGluSnFr to 1 mM glutamate addition and GCaMP6m to 5 μM ionomycin taken as bulk measurements of the cell cultures with plate readers (Tecan Spark for BLING and Biotek Cytation 5 for fluorescent readings; n=2 for BLING, n =3 for iGluSnFr, n=12 for GCaMP). FIG. 3C shows dose-dependent responses of BLING when using a plate reader (n =3). *=p<0.05, **=p<0.01, ***=p<0.001, ****=p<0.0001.

FIG. 4A shows an example trace of a single cell region of interest (ROI) showing the perfusion of Furimazine starting at 60 sec and the infusion of 1 mM glutamate at 120 sec. FIG. 4B shows dose responses of parental BLING 0.2 and the improved variant BLING 1.0 (n=4 for BLING 0.2, n =8 for BLING 1.0). FIG. 4C shows an image of background bioluminescence of BLING 1.0. FIG. 4D shows an image of bioluminescence of BLING 1.0 with 1 mM glutamate. *=p<0.05.

FIG. 5A shows a schematic of the experimental design for expression of BLING 1.0 at 2mm deep within the sensory cortex, with bicuculline injected locally to induce an acute seizure. FIG. 5B shows example images of bioluminescence before and after seizure induction. FIG. 5C shows an example trace of bioluminescent intensity in response to acute seizure induction.

FIG. 6A shows a schematic of a bioluminescent indicator variant for the neurotransmitter dopamine using a split luciferase and a neurotransmitter sensing/binding domain displayed on the cell surface. A library was developed with circularly permutated luciferases with varied linker lengths and luciferases inserted between the S5 and S6 transmembrane domains of the human dopamine receptor previously used to create Dlight creating luminescent dopamine indicators (DLume). FIG. 6B shows responses from the dopamine bioluminescent sensor library of linker mutants that were tested. FIG. 6C shows responses of DLume variants, where an improved DLume 3.2 with linker mutations had an approximately 20% increase in luminescence and was the brightest DLume variant.

FIG. 7A-B show protein schematics of exemplary bioluminescent BLIN variants specific to the neurotransmitter acetylcholine (Ach). FIG. 7C-D show protein schematics of exemplary bioluminescent BLIN variants specific to the neurotransmitter GABA.

FIG. 8 shows the measured bioluminescence response amplitude for the exemplary Ach and GABA sensors depicted in FIG. 7A-D in response to the respective neurotransmitters.

FIG. 9A-B show the design and luminescent responses of exemplary GABA and Ach sensors. FIG. 9A shows the design and luminescent response of an exemplary GABA sensor (BLIN-GABA 4.11) to GABA addition recorded with a plate reader. GABA BP=GABA Binding Protein. FIG. 9B shows the design and luminescent response of an exemplary Ach sensor (BLIN-Ach 4.10) to Ach addition recorded with a plate reader. Ach BP=Ach Binding Protein.

FIG. 10A-B show the design and luminescent response of exemplary Ach sensors. FIG. 10A shows the design of an exemplary BLIN-Ach sensor. Ach BP=Ach Binding Protein. FIG. 10B shows the luminescent response vs. background luminescence (i.e., noise) of an Ach sensor library. The arrow indicates the top Ach sensor variant from the library that exhibits the best signal to noise ratio.

FIG. 11 shows a schematic of a bioluminescent BLIN specific to the neurotransmitter serotonin (BLIN-Serotonin).

FIG. 12 shows a schematic of a bioluminescent sensor specific to glucose.

### DETAILED DESCRIPTION

While the disclosure has been described in connection with certain embodiments, it is to be understood that the disclosure is not to be limited to the disclosed embodiments and is intended to cover various modifications and equivalent arrangements included within the scope of the appended claims, which scope is to be accorded the broadest interpretation to encompass all such modifications and equivalent structures as is permitted under the law.

Unless otherwise defined, all technical and scientific terms used herein have the same meaning as commonly understood by one of ordinary skill in the art. For example, any nomenclatures used in connection with, and techniques of, cell and tissue culture, molecular biology, immunology, microbiology, genetics, and protein and nucleic acid chemistry and hybridization described herein are well known and commonly used in the art. In case of conflict, the present disclosure, including definitions, will control. Exemplary methods and materials are described below, although methods and materials similar or equivalent to those described herein can be used in practice or testing of the embodiments and aspects described herein.

As used herein, the terms “amino acid,” “nucleotide,” “nucleic acid,” “ribonucleic acid,” “deoxyribonucleic acid,” “polynucleotide,” “vector,” “polypeptide,” and “protein” have their common meanings as would be understood by a biochemist of ordinary skill in the art. Standard single letter nucleotides (A, C, G, T, U) and standard single letter amino acids (A, C, D, E, F, G, H, I, K, L, M, N, P, Q, R, S, T, V, W, or Y) are used herein. Nucleic acids may be single stranded or double stranded or may contain portions of both double stranded and single stranded sequence.

The nucleic acid may be DNA, both genomic and cDNA, RNA, or a hybrid thereof, where the nucleic acid may contain combinations of deoxyribo- and ribo-nucleotides, and combinations of bases including uracil, adenine, thymine, cytosine, guanine, inosine, xanthine, hypoxanthine, isocytosine and isoguanine. Nucleic acids may be obtained by chemical synthesis methods or by recombinant methods.

As used herein, “variants” can include, but are not limited to, those that include conservative amino acid (AA) substitution, SNP variants, degenerate variants, and biologically active portions of a gene. A “degenerate variant” as used herein refers to a variant that has a mutated nucleotide sequence, but still encodes the same polypeptide due to the redundancy of the genetic code. There are 20 naturally occurring amino acids; however, some of these share similar characteristics. For example, leucine and isoleucine are both aliphatic, branched, and hydrophobic. Similarly, aspartic acid and glutamic acid are both small and negatively charged. Conservative substitutions in proteins often have a smaller effect on function than non-conservative mutations. Although there are many ways to classify amino acids, they are often sorted into six main groups on the basis of their structure and the general chemical characteristics of their R groups. A mutation among the same class of amino acids is considered a conservative amino acid substitution.

The term “functional” when used in conjunction with “variant” or “fragment” refers to an entity or molecule which possess a biological activity that is substantially similar to a biological activity of the entity or molecule of which it is a variant or fragment thereof. In accordance with the present invention, a polynucleotide sequence encoding a recombinant bioluminescent polypeptide sensor may be modified, for example, to facilitate or improve identification, expression, isolation, storage and/or administration, so long as such modifications do not reduce its function to an unacceptable level.

As used herein, “substantial identity” of polynucleotide sequences means that a polynucleotide comprises a sequence that has at least 25% sequence identity compared to a reference sequence as determined using programs known in the art (e.g., Basic Local Alignment Search Tool (BLAST)). In preferred embodiments, percent identity can be any integer from 25% to 100%. More preferred embodiments include polynucleotide sequences that have at least about: 25%, 30%, 35%, 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, or 99% sequence identity compared to a reference sequence. These values can be appropriately adjusted to determine corresponding identity of proteins encoded by two nucleotide sequences by taking into account codon degeneracy, amino acid similarity, reading frame positioning, and the like. Accordingly, polynucleotides of the present invention encoding a protein or polypeptide of the present invention include nucleic acid sequences that have substantial identity to the nucleic acid sequences that encode the proteins or polypeptides of the present invention. Polynucleotides encoding a polypeptide comprising an amino acid sequence that has at least about: 25%, 30%, 35%, 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, or 99% sequence identity compared to a reference polypeptide sequence are also preferred.

As used herein, “substantial identity” of amino acid sequences (and of polypeptides having these amino acid sequences) means that an amino acid sequence comprises a sequence that has at least 25% sequence identity compared to a reference sequence as determined using programs known in the art (e.g., BLAST). In preferred embodiments, percent identity can be any integer from 25% to 100%. More preferred embodiments include amino acid or polypeptide sequences that have at least about: 25%, 30%, 35%, 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, or 99% sequence identity compared to a reference sequence. Polypeptides that are “substantially identical” share amino acid sequences except that residue positions which are not identical may differ by one or more conservative amino acid changes, as described above. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Exemplary conservative amino acid substitution groups include valine-leucine-isoleucine, phenylalanine-tyrosine, lysine-arginine, alanine-valine, aspartic acid-glutamic acid, and asparagine-glutamine. Accordingly, polypeptides or proteins, encoded by the polynucleotides of the present invention, include amino acid sequences that have substantial identity to the amino acid sequences of the reference polypeptide sequences.

As used herein, the terms such as “include,” “including,” “contain,” “containing,” “having,” and the like mean “comprising.” The present disclosure also contemplates other embodiments “comprising,” “consisting of,” and “consisting essentially of,” the embodiments or elements presented herein, whether explicitly set forth or not.

As used herein, the term “a,” “an,” “the” and similar terms used in the context of the disclosure (especially in the context of the claims) are to be construed to cover both the singular and plural unless otherwise indicated herein or clearly contradicted by the context. In addition, “a,” “an,” or “the” means “one or more” unless otherwise specified.

As used herein, the term “or” can be conjunctive or disjunctive.

As used herein, the term “substantially” means to a great or significant extent, but not completely.

As used herein, the term “about” or “approximately” as applied to one or more values of interest, refers to a value that is similar to a stated reference value, or within an acceptable error range for the particular value as determined by one of ordinary skill in the art, which will depend in part on how the value is measured or determined, such as the limitations of the measurement system. In one aspect, the term “about” refers to any values, including both integers and fractional components that are within a variation of up to ±10% of the value modified by the term “about.” Alternatively, “about” can mean within 3 or more standard deviations, per the practice in the art. Alternatively, such as with respect to biological systems or processes, the term “about” can mean within an order of magnitude, in some embodiments within 5-fold, and in some embodiments within 2-fold, of a value. As used herein, the symbol “˜” means “about” or “approximately.”

All ranges disclosed herein include both end points as discrete values as well as all integers and fractions specified within the range. For example, a range of 0.1-2.0 includes 0.1, 0.2, 0.3, 0.4 . . . 2.0. If the end points are modified by the term “about,” the range specified is expanded by a variation of up to ±10% of any value within the range or within 3 or more standard deviations, including the end points.

As used herein, the terms “active ingredient” or “active pharmaceutical ingredient” refer to a pharmaceutical agent, active ingredient, compound, or substance, compositions, or mixtures thereof, that provide a pharmacological, therapeutic, often beneficial, effect. In some embodiments, disclosed compositions may further comprise one or more pharmaceutically acceptable carriers or excipients. Example carriers may include, but are not limited to, liposomes, polymeric micelles, microspheres, microparticles, dendrimers, and/or nanoparticles. Example excipients may include, but are not limited to, buffering agents, salts, detergents, surfactants, acidifying agents, alkalizing agents, antimicrobial preservatives, antioxidants, chelating agents, and/or solubilizing agents.

As used herein, the terms “control,” or “reference” are used herein interchangeably. A “reference” or “control” level may be a predetermined value or range, which is employed as a baseline or benchmark against which to assess a measured result. “Control” also refers to control experiments or control cells. As used herein, the term “dose” denotes any form of an active ingredient formulation or composition, including cells, that contains an amount sufficient to initiate or produce an effect with at least one or more administrations. “Formulation” and “composition” are used interchangeably herein. As used herein, the term “administering” refers to the placement of an agent or a composition as disclosed herein into a subject by a method or route which results in at least partial localization of the agents or composition at a desired site. “Route of administration” may refer to any administration pathway known in the art, including but not limited to oral, intravenous (IV), topical, aerosol, nasal, via inhalation, anal, intra-anal, peri-anal, transmucosal, transdermal, parenteral, enteral, or local. “Parenteral” refers to a route of administration that is generally associated with injection, including intracranial, intraventricular, intrathecal, epidural, intradural, intraorbital, infusion (e.g., cardiac catheter infusion), intracapsular, intracardiac, intradermal, intramuscular, intraperitoneal, intrapulmonary, intraspinal, intrasternal, intrathecal, intrauterine, intravascular, intravenous (IV), intraarterial, subarachnoid, subcapsular, subcutaneous, transmucosal, or transtracheal. Via the parenteral route, the agent or composition may be in the form of solutions or suspensions for IV infusion or IV injection, or as lyophilized powders. Via the enteral route, the agent or composition can be in the form of capsules, gel capsules, tablets, sugar-coated tablets, syrups, suspensions, solutions, powders, granules, emulsions, microspheres or nanospheres or lipid vesicles or polymer vesicles allowing controlled release. Via the topical route, the agent or composition can be in the form of aerosol, lotion, cream, gel, ointment, suspensions, solutions, or emulsions. In one embodiment, the agent or composition may be provided in a powder form and mixed with a liquid, such as water, to form a beverage. In accordance with the present invention, “administering” can be self-administering. For example, it is considered “administering” when a subject consumes a composition as disclosed herein.

As used herein, the term “subject” refers to an animal. Typically, the subject is a mammal. A subject also refers to primates (e.g., humans, male or female; infant, adolescent, or adult), non-human primates, rats, mice, rabbits, pigs, cows, sheep, goats, horses, dogs, cats, fish, birds, and the like. In one embodiment, the subject is an animal. In another embodiment, the subject is a primate. In another embodiment, the subject is a human. In another embodiment, the subject is a non-human mammal.

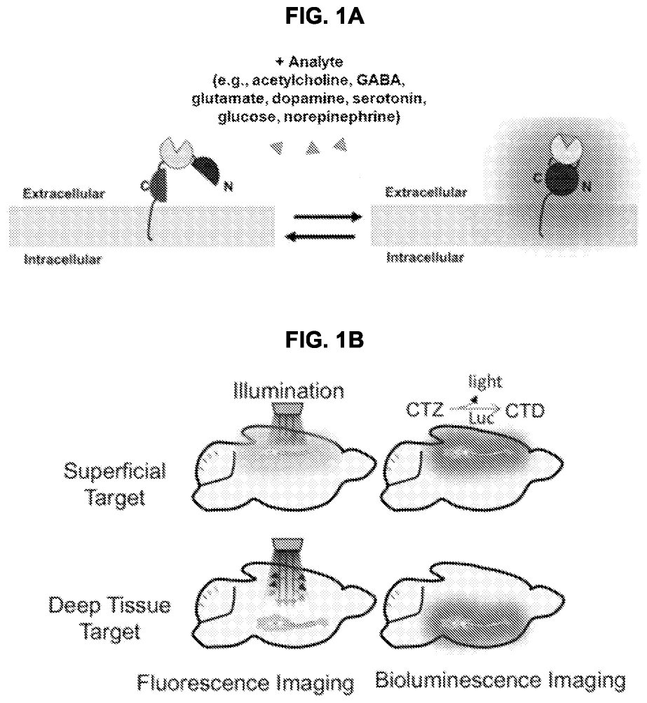

Described herein are different types of bioluminescent sensors/indicators for detecting various neurotransmitters and changes in membrane voltage to measure neuronal activity in vivo. In some embodiments, a series of bioluminescent Genetically Encoded Neurotransmitter Indicators (bGENIs) are described that may provide neuroscientists with a set of tools that can be used in lieu of physical collection and fluorescence detection approaches. These bGENIs are engineered as split luciferases having an analyte binding protein, such as a specific neurotransmitter binding protein, inserted in the middle of the split luciferase to form a single polypeptide chain. Upon binding of a neurotransmitter, the separate halves of the bioluminescent luciferase protein are brought together to create a functional bioluminescent protein which produces light upon exposure to its substrate, such as coelenterazine or furimazine (FIG. 1A-B). Voltage-sensitive variants may include bioluminescent proteins which are split in half and brought together upon a particular voltage change from a hyperpolarization state to a depolarization state.

In some embodiments, neurotransmitter-detecting bGENIs are defined as BioLuminescent Indicators of Neurotransmitter (BLIN) variants, based on an original BioLuminescent Indicator of the Neurotransmitter Glutamate (BLING) construct and its specific glutamate-binding variants. Glutamatergic neurotransmission is directly implicated in behavior, movement, mental health, pain perception, and addiction, making the neurotransmitter glutamate an attractive target for the development of bGENIs. The BLING sensors and subsequent BLIN variants specific to other neurotransmitters can be further adapted and engineered to create novel therapeutic agents for a variety of neurological disorders by acting on light-sensitive proteins (optogenetic actuators). Light-sensing proteins can control a variety of cellular processes, including membrane potential and gene expression. By pairing neurotransmitter light-emitting proteins to various light-sensing proteins, these cellular processes can be controlled in a more highly specific manner, dependent on neuronal activity.

The present disclosure provides, inter alia, genetically encoded recombinant bioluminescent polypeptide sensors comprising analyte-binding framework portions operably linked to signaling portions, wherein the signaling portions are oriented in relation to the analyte-binding framework portions at sites or amino acid positions that undergo a conformational change upon interaction of the framework portion with an analyte. In some embodiments, the analyte-binding framework portions comprise neurotransmitter binding domains, and the analyte is a neurotransmitter (e.g., acetylcholine, GABA, glutamate, dopamine, serotonin). In some embodiments, the signaling portions comprise a luminescent signaling domain (e.g., a luciferase polypeptide), where the luciferase polypeptide is split into two luciferase polypeptide domains, and wherein the analyte binding domain is present between the two luciferase polypeptide domains. Non-limiting exemplary luciferases may include Gaussia Luc, Renilla Luc, EkL9h, and alternate split sites in NanoLuc, as well as fusion combinations with mNeonGreen to shift the emission wavelength from blue to green. The NanoLuc luciferase is described by Dixon et al., ACS Chem. Biol., 11(2): 400-408 (2016), the entire contents of which are fully incorporated herein by reference. These luciferase variants emit luminescence at different wavelengths and may be used in combination with sensor/binding domains that are specific to any type of neurotransmitter.

One embodiment described herein is a recombinant bioluminescent polypeptide sensor comprising: (a) a luminescent signaling domain; (b) an analyte binding domain; and (c) one or more peptide linkers; wherein the luminescent signaling domain is oriented in relation to the analyte binding domain such that binding of an analyte to the analyte binding domain induces a conformational change in the luminescent signaling domain to generate a luminescent signal. In one aspect, the sensor may further comprise (d) one or more cellular trafficking peptides such as membrane trafficking peptides. In another aspect, the luminescent signaling domain is allosterically regulated by the analyte binding domain such that signaling from the luminescent signaling domain is altered upon interaction of the analyte binding domain with the analyte. In another aspect, signaling by the luminescent signaling domain is proportional to the level of interaction between the analyte binding domain and the analyte. In another aspect, the luminescent signaling domain comprises a luciferase polypeptide. In another aspect, the luciferase polypeptide is split into two luciferase polypeptide domains, and wherein the analyte binding domain is present between the two luciferase polypeptide domains. In another aspect, the luciferase polypeptide emits luminescence at a wavelength ranging from about 450 nm to about 540 nm. In another aspect, the analyte binding domain comprises a neurotransmitter binding domain. In another aspect, the neurotransmitter binding domain comprises one or more glutamate binding domains, or functional variants, mutants, or fragments thereof. In another aspect, the neurotransmitter binding domain binds specifically to the neurotransmitter glutamate. In another aspect, the sensor is encoded by a polynucleotide sequence having at least 90-99% identity to any one of SEQ ID NO: 1, 3, 5, or 7. In another aspect, the sensor is encoded by a polynucleotide sequence selected from any one of SEQ ID NO: 1, 3, 5, or 7. In another aspect, the sensor has an amino acid sequence having at least 90-99% identity to any one of SEQ ID NO: 2, 4, 6, or 8. In another aspect, the sensor has an amino acid sequence selected from any one of SEQ ID NO: 2, 4, 6, or 8. In another aspect, the neurotransmitter binding domain comprises one or more dopamine binding domains, or functional variants, mutants, or fragments thereof. In another aspect, the neurotransmitter binding domain binds specifically to the neurotransmitter dopamine. In another aspect, the sensor is encoded by a polynucleotide sequence having at least 90-99% identity to SEQ ID NO: 9. In another aspect, the sensor is encoded by a polynucleotide sequence selected from SEQ ID NO: 9. In another aspect, the sensor has an amino acid sequence having at least 90-99% identity to SEQ ID NO: 10. In another aspect, the sensor has an amino acid sequence selected from SEQ ID NO: 10. In another aspect, the neurotransmitter binding domain comprises one or more acetylcholine binding domains, or functional variants, mutants, or fragments thereof. In another aspect, the neurotransmitter binding domain binds specifically to the neurotransmitter acetylcholine. In another aspect, the sensor is encoded by a polynucleotide sequence having at least 90-99% identity to any one of SEQ ID NO: 11, 13, or 15. In another aspect, the sensor is encoded by a polynucleotide sequence selected from any one of SEQ ID NO: 11, 13, or 15. In another aspect, the sensor has an amino acid sequence having at least 90-99% identity to any one of SEQ ID NO: 12, 14, or 16. In another aspect, the sensor has an amino acid sequence selected from any one of SEQ ID NO: 12, 14, or 16. In another aspect, the neurotransmitter binding domain comprises one or more GABA binding domains, or functional variants, mutants, or fragments thereof. In another aspect, the neurotransmitter binding domain binds specifically to the neurotransmitter GABA. In another aspect, the sensor is encoded by a polynucleotide sequence having at least 90-99% identity to any one of SEQ ID NO: 17, 19, or 21. In another aspect, the sensor is encoded by a polynucleotide sequence selected from any one of SEQ ID NO: 17, 19, or 21. In another aspect, the sensor has an amino acid sequence having at least 90-99% identity to any one of SEQ ID NO: 18, 20, or 22. In another aspect, the sensor has an amino acid sequence selected from any one of SEQ ID NO: 18, 20, or 22. In another aspect, the neurotransmitter binding domain comprises one or more serotonin binding domains, or functional variants, mutants, or fragments thereof. In another aspect, the neurotransmitter binding domain binds specifically to the neurotransmitter serotonin. In another aspect, the sensor is encoded by a polynucleotide sequence having at least 90-99% identity to SEQ ID NO: 23. In another aspect, the sensor is encoded by a polynucleotide sequence selected from SEQ ID NO: 23. In another aspect, the sensor has an amino acid sequence having at least 90-99% identity to SEQ ID NO: 24. In another aspect, the sensor has an amino acid sequence selected from SEQ ID NO: 24. In another aspect, the analyte binding domain comprises one or more glucose binding domains, or functional variants, mutants, or fragments thereof. In another aspect, the analyte binding domain binds specifically to glucose. In another aspect, the sensor is encoded by a polynucleotide sequence having at least 90-99% identity to SEQ ID NO: 25. In another aspect, the sensor is encoded by a polynucleotide sequence selected from SEQ ID NO: 25. In another aspect, the sensor has an amino acid sequence having at least 90-99% identity to SEQ ID NO: 26. In another aspect, the sensor has an amino acid sequence selected from SEQ ID NO: 26.

Another embodiment described herein is a vector comprising a polynucleotide sequence encoding any one of the recombinant bioluminescent polypeptide sensors disclosed herein. In one aspect, the vector is selected from a viral vector, a plasmid expression vector, an adeno-associated virus (AAV) vector, a recombinant AAV (rAAV) vector, a single-stranded AAV vector, a double-stranded AAV vector, or a self-complementary AAV (scAAV) vector. In another aspect, the vector is an AAV vector of a serotype selected from AAV1, AAV2, AAV3, AAV4, AAV5, AAV6, AAV7, AAV8, AAV9, AAV10, AAV11, AAV12, or a hybrid serotype thereof. In another aspect, the vector is a pcDNA3.1 plasmid expression vector.

Another embodiment described herein is a cell comprising any one of the vectors disclosed herein. In one aspect, the cell is a mammalian cell from a human, mouse, rat, dog, cat, pig, goat, rabbit, cow, horse, or other non-human primate.

Another embodiment described herein is a method for detecting one or more analytes in a subject, the method comprising measuring a level of luminescence emitted by any one of the recombinant bioluminescent polypeptide sensors disclosed herein and correlating the measured level of luminescence with the presence of the one or more analytes in the subject. In one aspect, the recombinant bioluminescent polypeptide sensor is encoded and expressed from a polynucleotide sequence that is administered to the subject. In another aspect, the polynucleotide sequence has at least 90-99% identity to any one of SEQ ID NO: 1, 3, 5, 7, 9, 11, 13, 15, 17, 19, 21, 23, or 25. In another aspect, the polynucleotide sequence is selected from any one of SEQ ID NO: 1, 3, 5, 7, 9, 11, 13, 15, 17, 19, 21, 23, or 25. In another aspect, the recombinant bioluminescent polypeptide sensor has an amino acid sequence having at least 90-99% identity to any one of SEQ ID NO: 2, 4, 6, 8, 10, 12, 14, 16, 18, 20, 22, 24, or 26. In another aspect, the recombinant bioluminescent polypeptide sensor has an amino acid sequence selected from any one of SEQ ID NO: 2, 4, 6, 8, 10, 12, 14, 16, 18, 20, 22, 24, or 26. In another aspect, the subject is a human. In another aspect, the subject is an animal. In another aspect, the subject is a non-human mammal.

Another embodiment described herein is the use of any one of the recombinant bioluminescent polypeptide sensors disclosed herein to detect one or more analytes in a subject. Another embodiment described herein is a means or process for manufacturing one or more of the polynucleotide sequences described herein or the recombinant bioluminescent polypeptide sensors encoded by the polynucleotide sequences described herein, the process comprising: transforming or transfecting a cell with a nucleic acid comprising a polynucleotide sequence described herein; growing the cells; optionally isolating additional quantities of the polynucleotide sequence described herein; inducing expression of a polypeptide encoded by the polynucleotide sequence described herein; and isolating the polypeptide encoded by the polynucleotide sequence described herein.

Another embodiment described herein is a polynucleotide sequence or a polypeptide encoded by the polynucleotide sequence produced by the methods or the means described herein.

Another embodiment described herein is the use of an effective amount of a polypeptide encoded by one or more of the polynucleotide sequences described herein.

Another embodiment described herein is a polynucleotide vector comprising one or more polynucleotide sequences described herein.

Another embodiment described herein is a cell comprising one or more polynucleotide sequences described herein or a polynucleotide vector described herein.

Another embodiment described herein is a research tool comprising the polynucleotide sequences described herein.

Another embodiment described herein is a research tool comprising a polypeptide encoded by a polynucleotide sequence described herein.

Another embodiment described herein is a reagent comprising the polynucleotide sequences described herein.

Another embodiment described herein is a reagent comprising a polypeptide encoded by a polynucleotide sequence described herein.

Further embodiments described herein include nucleic acid molecules comprising polynucleotides having nucleotide sequences about 50%, 55%, 60%, 65%, 70%, 75%, 80%, 85%, 90%, 91%, 92%, 93%, 94%, 95%, 96%, 97%, 98%, or 99% identical, and more preferably at least about 90-99% or 100% identical to (a) nucleotide sequences, or degenerate, homologous, or codon-optimized variants thereof, having the polynucleotide sequences in SEQ ID NO: 1, 3, 5, 7, 9, 11, 13, 15, 17, 19, 21, 23, or 25; or (b) nucleotide sequences capable of hybridizing to the complement of any of the nucleotide sequences in (a).

By a polynucleotide having a nucleotide sequence at least, for example, 90-99% “identical” to a reference nucleotide sequence intended that the nucleotide sequence of the polynucleotide be identical to the reference sequence except that the polynucleotide sequence can include up to about 10 to 1 point mutations, additions, or deletions per each 100 nucleotides of the reference nucleotide sequence.

In other words, to obtain a polynucleotide having a nucleotide sequence about at least 90-99% identical to a reference nucleotide sequence, up to 10% of the nucleotides in the reference sequence can be deleted, added, or substituted, with another nucleotide, or a number of nucleotides up to 10% of the total nucleotides in the reference sequence can be inserted into the reference sequence. These mutations of the reference sequence can occur at the 5′- or 3′-terminal positions of the reference nucleotide sequence or anywhere between those terminal positions, interspersed either individually among nucleotides in the reference sequence or in one or more contiguous groups within the reference sequence. The same is applicable to polypeptide sequences about at least 90-99% identical to a reference polypeptide sequence.

As noted above, two or more polynucleotide sequences can be compared by determining their percent identity. Two or more amino acid sequences likewise can be compared by determining their percent identity. The percent identity of two sequences, whether nucleic acid or peptide sequences, is generally described as the number of exact matches between two aligned sequences divided by the length of the shorter sequence and multiplied by 100. An approximate alignment for nucleic acid sequences is provided by the local homology algorithm of Smith and Waterman, Advances in Applied Mathematics 2: 4 82-489 (1981). This algorithm can be extended to use with peptide sequences using the scoring matrix developed by Dayhoff, Atlas of Protein Sequences and Structure, M. O. Dayhoff ed., 5 suppl. 3:353-358, National Biomedical Research Foundation, Washington, D.C., USA, and normalized by Gribskov, Nucl. Acids Res. 14 (6): 6745-6763 (1986).

The polynucleotides described herein may include variants having substitutions, deletions, and/or additions that can involve one or more nucleotides. The variants can be altered in coding regions, non-coding regions, or both. Alterations in the coding regions can produce conservative or non-conservative amino acid substitutions, deletions, or additions. Especially preferred among these are silent substitutions, additions, and deletions, which do not alter the properties and activities of binding.

The polynucleotides described herein include those encoding any mutations, variations, substitutions, additions, deletions, and particular examples of the polypeptides described herein. For example, guidance concerning how to make phenotypically silent amino acid substitutions is provided in Bowie, J. U. et al., “Deciphering the Message in Protein Sequences: Tolerance to Amino Acid Substitutions,” Science 247:1306-1310 (1990), wherein the authors indicate that proteins are surprisingly tolerant of amino acid substitutions.

As described herein, in many cases the amino acid substitutions, mutations, additions, or deletions of the disclosed polypeptides are preferably of a minor nature, such as conservative amino acid substitutions that do not significantly affect the folding or functional activity of the protein or additions or deletions to the N-or C-termini. Of course, the number of amino acid substitutions, additions, or deletions a skilled artisan would make depends on many factors, including those described herein. Generally, the number of substitutions, additions, or deletions for any given polypeptide will not be more than about 100, 90, 80, 70, 60, 50, 40, 30, 25, 20, 19, 18, 17, 16, 15, 14, 13, 12, 11, 10, 9, 8, 7, 5, 6, 4, 3, 2, or 1.

It will be apparent to one of ordinary skill in the relevant art that suitable modifications and adaptations to the compositions, formulations, methods, processes, and applications described herein can be made without departing from the scope of any embodiments or aspects thereof. The compositions and methods provided are exemplary and are not intended to limit the scope of any of the specified embodiments. All of the various embodiments, aspects, and options disclosed herein can be combined in any variations or iterations. The scope of the compositions, formulations, methods, and processes described herein include all actual or potential combinations of embodiments, aspects, options, examples, and preferences herein described. The exemplary compositions and formulations described herein may omit any component, substitute any component disclosed herein, or include any component disclosed elsewhere herein. The ratios of the mass of any component of any of the compositions or formulations disclosed herein to the mass of any other component in the formulation or to the total mass of the other components in the formulation are hereby disclosed as if they were expressly disclosed.

Should the meaning of any terms in any of the patents or publications incorporated by reference conflict with the meaning of the terms used in this disclosure, the meanings of the terms or phrases in this disclosure are controlling. Furthermore, the foregoing discussion discloses and describes merely exemplary embodiments. All patents and publications cited herein are incorporated by reference herein for the specific teachings thereof.

Various embodiments and aspects of the inventions described herein are summarized by the following clauses:

Clause 1. A recombinant bioluminescent polypeptide sensor comprising:
(a) a luminescent signaling domain;
(b) an analyte binding domain; and
(c) one or more peptide linkers;
wherein the luminescent signaling domain is oriented in relation to the analyte binding domain such that binding of an analyte to the analyte binding domain induces a conformational change in the luminescent signaling domain to generate a luminescent signal.

Clause 2. The sensor of clause 1, further comprising (d) one or more cellular trafficking peptides comprising membrane trafficking peptides.

Clause 3. The sensor of clause 1 or 2, wherein the luminescent signaling domain is allosterically regulated by the analyte binding domain such that signaling from the luminescent signaling domain is altered upon interaction of the analyte binding domain with the analyte.

Clause 4. The sensor of any one of clauses 1-3, wherein signaling by the luminescent signaling domain is proportional to the level of interaction between the analyte binding domain and the analyte.

Clause 5. The sensor of any one of clauses 1-4, wherein the luminescent signaling domain comprises a luciferase polypeptide.

Clause 6. The sensor of any one of clauses 1-5, wherein the luciferase polypeptide is split into two luciferase polypeptide domains, and wherein the analyte binding domain is present between the two luciferase polypeptide domains.

Clause 7. The sensor of any one of clauses 1-6, wherein the luciferase polypeptide emits luminescence at a wavelength ranging from about 450 nm to about 540 nm.

Clause 8. The sensor of any one of clauses 1-7, wherein the analyte binding domain comprises a neurotransmitter binding domain.

Clause 9. The sensor of any one of clauses 1-8, wherein the neurotransmitter binding domain comprises one or more glutamate binding domains, or functional variants, mutants, or fragments thereof.

Clause 10. The sensor of any one of clauses 1-9, wherein the neurotransmitter binding domain binds specifically to the neurotransmitter glutamate.

Clause 11. The sensor of any one of clauses 1-10, wherein the sensor is encoded by a polynucleotide sequence having at least 90-99% identity to any one of SEQ ID NO: 1, 3, 5, or 7.

Clause 12. The sensor of any one of clauses 1-11, wherein the sensor is encoded by a polynucleotide sequence selected from any one of SEQ ID NO: 1, 3, 5, or 7.

Clause 13. The sensor of any one of clauses 1-12, wherein the sensor has an amino acid sequence having at least 90-99% identity to any one of SEQ ID NO: 2, 4, 6, or 8.

Clause 14. The sensor of any one of clauses 1-13, wherein the sensor has an amino acid sequence selected from any one of SEQ ID NO: 2, 4, 6, or 8.

Clause 15. The sensor of any one of clauses 1-14, wherein the neurotransmitter binding domain comprises one or more dopamine binding domains, or functional variants, mutants, or fragments thereof.

Clause 16. The sensor of any one of clauses 1-15, wherein the neurotransmitter binding domain binds specifically to the neurotransmitter dopamine.

Clause 17. The sensor of any one of clauses 1-16, wherein the sensor is encoded by a polynucleotide sequence having at least 90-99% identity to SEQ ID NO: 9.

Clause 18. The sensor of any one of clauses 1-17, wherein the sensor is encoded by a polynucleotide sequence selected from SEQ ID NO: 9.

Clause 19. The sensor of any one of clauses 1-18, wherein the sensor has an amino acid sequence having at least 90-99% identity to SEQ ID NO: 10.

Clause 20. The sensor of any one of clauses 1-19, wherein the sensor has an amino acid sequence selected from SEQ ID NO: 10.

Clause 21. The sensor of any one of clauses 1-20, wherein the neurotransmitter binding domain comprises one or more acetylcholine binding domains, or functional variants, mutants, or fragments thereof.

Clause 22. The sensor of any one of clauses 1-21, wherein the neurotransmitter binding domain binds specifically to the neurotransmitter acetylcholine.

Clause 23. The sensor of any one of clauses 1-22, wherein the sensor is encoded by a polynucleotide sequence having at least 90-99% identity to any one of SEQ ID NO: 11, 13, or 15.

Clause 24. The sensor of any one of clauses 1-23, wherein the sensor is encoded by a polynucleotide sequence selected from any one of SEQ ID NO: 11, 13, or 15.

Clause 25. The sensor of any one of clauses 1-24, wherein the sensor has an amino acid sequence having at least 90-99% identity to any one of SEQ ID NO: 12, 14, or 16.

Clause 26. The sensor of any one of clauses 1-25, wherein the sensor has an amino acid sequence selected from any one of SEQ ID NO: 12, 14, or 16.

Clause 27. The sensor of any one of clauses 1-26, wherein the neurotransmitter binding domain comprises one or more GABA binding domains, or functional variants, mutants, or fragments thereof.

Clause 28. The sensor of any one of clauses 1-27, wherein the neurotransmitter binding domain binds specifically to the neurotransmitter GABA.

Clause 29. The sensor of any one of clauses 1-28, wherein the sensor is encoded by a polynucleotide sequence having at least 90-99% identity to any one of SEQ ID NO: 17, 19, or 21.

Clause 30. The sensor of any one of clauses 1-29, wherein the sensor is encoded by a polynucleotide sequence selected from any one of SEQ ID NO: 17, 19, or 21.

Clause 31. The sensor of any one of clauses 1-30, wherein the sensor has an amino acid sequence having at least 90-99% identity to any one of SEQ ID NO: 18, 20, or 22.

Clause 32. The sensor of any one of clauses 1-31, wherein the sensor has an amino acid sequence selected from any one of SEQ ID NO: 18, 20, or 22.

Clause 33. The sensor of any one of clauses 1-32, wherein the neurotransmitter binding domain comprises one or more serotonin binding domains, or functional variants, mutants, or fragments thereof.

Clause 34. The sensor of any one of clauses 1-33, wherein the neurotransmitter binding domain binds specifically to the neurotransmitter serotonin.

Clause 35. The sensor of any one of clauses 1-34, wherein the sensor is encoded by a polynucleotide sequence having at least 90-99% identity to SEQ ID NO: 23.

Clause 36. The sensor of any one of clauses 1-35, wherein the sensor is encoded by a polynucleotide sequence selected from SEQ ID NO: 23.

Clause 37. The sensor of any one of clauses 1-36, wherein the sensor has an amino acid sequence having at least 90-99% identity to SEQ ID NO: 24.

Clause 38. The sensor of any one of clauses 1-37, wherein the sensor has an amino acid sequence selected from SEQ ID NO: 24.

Clause 39. The sensor of any one of clauses 1-38, wherein the analyte binding domain comprises one or more glucose binding domains, or functional variants, mutants, or fragments thereof.

Clause 40. The sensor of any one of clauses 1-39, wherein the analyte binding domain binds specifically to glucose.

Clause 41. The sensor of any one of clauses 1-40, wherein the sensor is encoded by a polynucleotide sequence having at least 90-99% identity to SEQ ID NO: 25.

Clause 42. The sensor of any one of clauses 1-41, wherein the sensor is encoded by a polynucleotide sequence selected from SEQ ID NO: 25.

Clause 43. The sensor of any one of clauses 1-42, wherein the sensor has an amino acid sequence having at least 90-99% identity to SEQ ID NO: 26.

Clause 44. The sensor of any one of clauses 1-43, wherein the sensor has an amino acid sequence selected from SEQ ID NO: 26.

Clause 45. A vector comprising a polynucleotide sequence encoding the recombinant bioluminescent polypeptide sensor of any one of clauses 1-44.

Clause 46. The vector of clause 45, wherein the vector is selected from a viral vector, a plasmid expression vector, an adeno-associated virus (AAV) vector, a recombinant AAV (rAAV) vector, a single-stranded AAV vector, a double-stranded AAV vector, or a self-complementary AAV (scAAV) vector.

Clause 47. The vector of clause 45 or 46, wherein the vector is an AAV vector of a serotype selected from AAV1, AAV2, AAV3, AAV4, AAV5, AAV6, AAV7, AAV8, AAV9, AAV10,AAV11, AAV12, or a hybrid serotype thereof.

Clause 48. The vector of any one of clauses 45-47, wherein the vector is a pcDNA3.1 plasmid expression vector.

Clause 49. A cell comprising the vector of any one of clauses 45-48.

Clause 50. The cell of clause 49, wherein the cell is a mammalian cell from a human, mouse, rat, dog, cat, pig, goat, rabbit, cow, horse, or other non-human primate.

Clause 51. A method for detecting one or more analytes in a subject, the method comprising measuring a level of luminescence emitted by the recombinant bioluminescent polypeptide sensor of any one of clauses 1-44 and correlating the measured level of luminescence with the presence of the one or more analytes in the subject.

Clause 52. The method of clause 51, wherein the recombinant bioluminescent polypeptide sensor is encoded and expressed from a polynucleotide sequence that is administered to the subject.

Clause 53. The method of clause 51 or 52, wherein the polynucleotide sequence has at least 90-99% identity to any one of SEQ ID NO: 1, 3, 5, 7, 9, 11, 13, 15, 17, 19, 21, 23, or 25.

Clause 54. The method of any one of clauses 51-53, wherein the polynucleotide sequence is selected from any one of SEQ ID NO: 1, 3, 5, 7, 9, 11, 13, 15, 17, 19, 21, 23, or 25.

Clause 55. The method of any one of clauses 51-54, wherein the recombinant bioluminescent polypeptide sensor has an amino acid sequence having at least 90-99% identity to any one of SEQ ID NO: 2, 4, 6, 8, 10, 12, 14, 16, 18, 20, 22, 24, or 26.

Clause 56. The method of any one of clauses 51-55, wherein the recombinant bioluminescent polypeptide sensor has an amino acid sequence selected from any one of SEQ ID NO: 2, 4, 6, 8, 10, 12, 14, 16, 18, 20, 22, 24, or 26.

Clause 57. The method of any one of clauses 51-56, wherein the subject is a human.

Clause 58. The method of any one of clauses 51-57, wherein the subject is an animal.

Clause 59. The method of any one of clauses 51-58, wherein the subject is a non-human mammal.

Clause 60. Use of the recombinant bioluminescent polypeptide sensor of any one of clauses 1-44 to detect one or more analytes in a subject.

### EXAMPLES

### Example 1

### Materials and Methods

### Bioluminescent Sensor Design

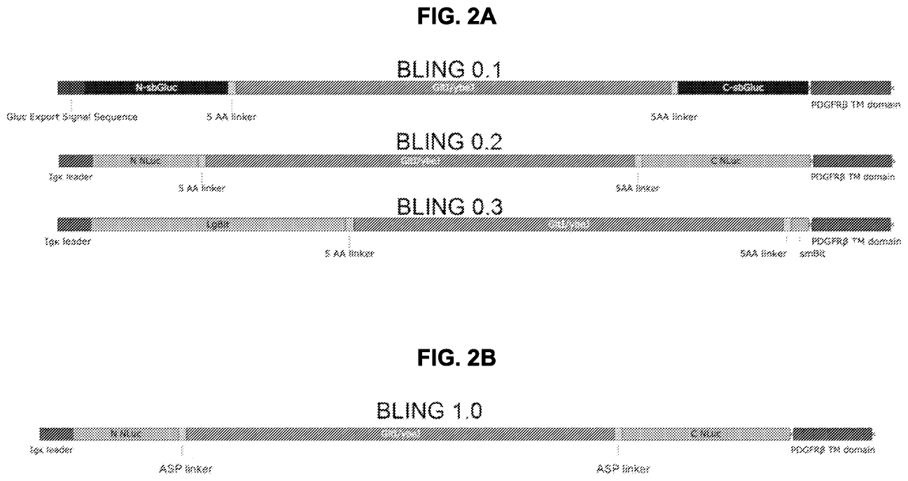

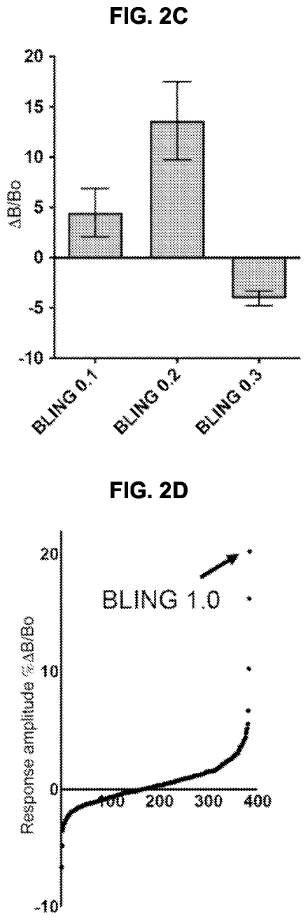

The initial three glutamate-specific BLING variants were constructed using Glt1, IgK leader, and PDFGRβ sequences (GenBank EU42295) synthesized as a Gblock or oligos used for PCR and assembled into a pcDNA 3.1 vector using Gibson Assembly (Neb HiFi) with their respective luciferase fragments. IgK leader and PDFGRβ sequences are used for membrane display on the cell surface. BLING 0.1 was generated with the sbGluc using the split site at amino acids 105-106, including the 17 amino acid native secretion signal. BLING 0.2 was generated with the NanoLuc split site at amino acids 66-67, with an Igk leader sequence for cell surface display. BLING 0.3 was generated with the NanoLuc large and small bits, split at site 159-160 (FIG. 2A-B). HEK cells plated on Poly-D-Lysine coated white 96 well plates and grown to 50-70% confluency were transfected with 0.5 uL Lipofectamine 2000 per mL Opti MEM and 100 ng DNA per well using 20 μL of the transfection mix per well. Initial testing was conducted using 5 UM hCTZ (Nanolight Technologies #301) in FluoroBrite media (Thermo), where the media was changed 15 minutes prior to measurement to allow the reaction to stabilize. Plates were read using a BioTek Cytation 5, where 10 μL of a 20 mM glutamate stock was injected into 190 μL media for a final concentration of 1 mM glutamate.

### Library Construction and Screening

Assembly products were digested with Dpnl to eliminate any plasmid template material, eliminating background colonies, and electroporated into Top10 cells (Thermo). Colonies were then grown in deep 96 well plates in 1.5 mL LB media overnight and miniprepped in 96 well format (Biobasic #B814152-0005). Each plasmid was transfected into HEK cells grown in Poly-D-Lysine coated white 384 well plates in quadruplicate to generate an average for each variant tested. A transfection master mix was prepared with 21 mL Opti MEM with 100 μL Lipofectamine 2000, distributed into four 96 well PCR plates at 50 μL per well, and 5 μL DNA from the 96 well mini preps was added to the transfection master mix with mini prep DNA yields ranging from 100-200 ng/μL. Testing was done two days later using 5 μM hCTZ in FluoroBrite media, where the media was changed 15 minutes prior to measurement to allow the reaction to stabilize. Plates were read using a BioTek Cytation 5, where 5 μL of a 20 mM glutamate stock was injected into 95 μL media for a final concentration of 1 mM glutamate.

### Characterization

The top BLING variant from the linker library was termed BLING 1.0 (Addgene plasmid: 171647) and further characterized and compared to the parental construct, BLING 0.2. HEK cells plated on white 96 well plates and grown to 50-70% confluency were transfected with 0.5 μL Lipofectamine 2000 per mL Opti MEM with 100 ng DNA per well using 20 μL of the transfection mix per well. Measurements were taken with 5 μM hCTZ in FluoroBrite media, where the media was changed 15 minutes prior to measurement on a Tecan spark for bioluminescence. GCaMP6 and iGluSnFr measurements were recorded with a Biotek Cytation 5 using 1 μM glutamate or 5 mM ionomycin, respectively. The concentration dependent experiment was conducted in HBSS with magnesium and calcium in 10 mM HEPES buffer and 1 mM hCTZ as it was found that these conditions provided less variability in measurements. Statistical analysis was done using a one-way ANOVA or two-way repeated measures with Bonferroni post-hoc (n=2-3 per group). The DNA and amino acid sequences of the different BLING glutamate constructs are shown in Table 1.

TABLE 1
BLING (Glutamate) Variant Polynucleotide and Amino Acid Sequences

| | |
|---|---|
| BLING | GACGGATCGGGAGATCTCCCGATCCCCTATGGTCGACTCTCAGTACAATCTGCTCTGATGCCGCA |
| 0.1 | TAGTTAAGCCAGTATCTGCTCCCTGCTTGTGTGTTGGAGGTCGCTGAGTAGTGCGCGAGCAAAAT |
| DNA | TTAAGCTACAACAAGGCAAGGCTTGACCGACAATTGCATGAAGAATCTGCTTAGGGTTAGGCGTT |
| (SEQ | TTGCGCTGCTTCGCGATGTACGGGCCAGATATACGCGTTGACATTGATTATTGACTAGTTATTAA |
| ID | TAGTAATCAATTACGGGGTCATTAGTTCATAGCCCATATATGGAGTTCCGCGTTACATAACTTAC |
| NO: | GGTAAATGGCCCGCCTGGCTGACCGCCCAACGACCCCCGCCCATTGACGTCAATAATGACGTATG |
| 1) | TTCCCATAGTAACGCCAATAGGGACTTTCCATTGACGTCAATGGGTGGACTATTTACGGTAAACT |
| | GCCCACTTGGCAGTACATCAAGTGTATCATATGCCAAGTACGCCCCCTATTGACGTCAATGACGG |
| | TAAATGGCCCGCCTGGCATTATGCCCAGTACATGACCTTATGGGACTTTCCTACTTGGCAGTACA |
| | TCTACGTATTAGTCATCGCTATTACCATGGTGATGCGGTTTTGGCAGTACATCAATGGGCGTGGA |
| | TAGCGGTTTGACTCACGGGGATTTCCAAGTCTCCACCCCATTGACGTCAATGGGAGTTTGTTTTG |
| | GCACCAAAATCAACGGGACTTTCCAAAATGTCGTAACAACTCCGCCCCATTGACGCAAATGGGCG |
| | GTAGGCGTGTACGGTGGGAGGTCTATATAAGCAGAGCTCTCTGGCTAACTAGAGAACCCACTGCT |
| | TACTGGCTTATCGAAATTAATACGACTCACTATAGGGAGACCCAAGCTGGCTAGTTAAGCTTGCC |
| | ATGGGAGTCAAAGTTCTGTTTGCCCTGATCTGCATCGCTGTGGCCGAGGCCAAGCCCACCGAGAA |
| | CAACGAAGACTTCAACATCGTGGCCGTGGCCAGCAACTTCGCGACCACGGATCTCGATGCTGACC |
| | GCGGGAAGTTGCCCGGCAAGAAGCTGCCGCTGGAGGTGCTCAAAGAGCTGGAAGCCAATGCCCGG |
| | AAAGCTGGCTGCACCAGGGGCTGTCTGATCTGCCTGTCCCACATCAAGTGCACGCCCAAGATGAA |
| | GAAGTTCATCCCAGGACGCTGCCACACCTACGAAGGCGACAAAGAGTCCGCACAGGGAGGAGGCG |
| | GTACCAGTACTCTGGACAAAATCGCCAAAAACGGTGTGATTGTCGTCGGTCACCGTGAATCTTCA |
| | GTGCCTTTCTCTTATTACGACAATCAGCAAAAAGTGGTGGGTTACTCGCAGGATTACTCCAACGC |
| | CATTGTTGAAGCAGTGAAAAAGAAACTCAACAAACCGGACTTGCAGGTAAAACTGATTCCGATTA |
| | CAACACAAAACCGTATTCCACTGCTGCAAAACGGCACTTTCGATTTTGAATGTGGTTCTACCACC |
| | AACAACGTCGAACGCCAAAAACAGGCGGCTTTCTCTGACACTATTTTCGTGGTCGGTACGCGCCT |
| | GTTGACCAAAAAGGGTGGCGATATCAAAGATTTTGCCAACCTGAAAGACAAAGCCGTAGTCGTCA |
| | CTTCCGGCACTACCTCTGAAGTTTTGCTCAACAAACTGAATGAAGAGCAAAAAATGAATATGCGC |
| | ATCATCAGCGCCAAAGATCACGGTGACTCTTTCCGCACCCTGGAAAGCGGTCGTGCCGTTGCCTT |
| | TATGATGGATGACGCTCTGCTGGCCGGTGAACGTGCGAAAGCGAAGAAACCAGACAACTGGGAAA |
| | TCGTCGGCAAGCCGCAGTCTCAGGAGGCCTACGGTTGTATGTTGCGTAAAGATGATCCGCAGTTC |
| | AAAAAGCTGATGGATGACACCATCGCTCAGGTGCAGACCTCCGGTGAAGCGGAAAAATGGTTTGA |
| | TAAGTGGTTCAAAAATCCAATTCCGCCGAAAAACCTGAACATGAATTTCGAACTGTCAGACGAAA |
| | TGAAAGCACTGTTCAAAGAACCGAATGACGGATCCGGAGGCGGCGGCGGCATAGGCGAGGCGATC |
| | GTCGACATTCCTGAGATTCCTGGGTTCAAGGACTTGGAGCCCCTGGAGCAGTTCATCGCACAGGT |
| | CGATCTGTGTGTGGACTGCACAACTGGCTGCCTCAAAGGGCTTGCCAACGTGCAGTGTTCTGACC |
| | TGCTCAAGAAGTGGCTGCCGCAACGCTGTGCGACCTTTGCCAGCAAGATCCAGGGCCAGGTGGAC |
| | AAGATCAAGGGGGCCGGTGGTGACCTCGAGGCTGTGGGCCAGGACACGCAGGAGGTCATCGTGGT |
| | GCCACACTCCTTGCCCTTTAAGGTGGTGGTGATCTCAGCCATCCTGGCCCTGGTGGTGCTCACCA |
| | TCATCTCCCTTATCATCCTCATCATGCTTTGGCAGAAGAAGCCACGTTAGTAACATATGCTCTAG |
| | AGGGCCCGCGGTTCGAAGGTAAGCCTATCCCTAACCCTCTCCTCGGTCTCGATTCTACGCGTACC |
| | GGTCATCATCACCATCACCATTGAGTTTAAACCCGCTGATCAGCCTCGACTGTGCCTTCTAGTTG |
| | CCAGCCATCTGTTGTTTGCCCCTCCCCCGTGCCTTCCTTGACCCTGGAAGGTGCCACTCCCACTG |
| | TCCTTTCCTAATAAAATGAGGAAATTGCATCGCATTGTCTGAGTAGGTGTCATTCTATTCTGGGG |
| | GGTGGGGTGGGGCAGGACAGCAAGGGGGAGGATTGGGAAGACAATAGCAGGCATGCTGGGGATGC |
| | GGTGGGCTCTATGGCTTCTGAGGCGGAAAGAACCAGCTGGGGCTCTAGGGGGTATCCCCACGCGC |
| | CCTGTAGCGGCGCATTAAGCGCGGCGGGTGTGGTGGTTACGCGCAGCGTGACCGCTACACTTGCC |
| | AGCGCCCTAGCGCCCGCTCCTTTCGCTTTCTTCCCTTCCTTTCTCGCCACGTTCGCCGGCTTTCC |
| | CCGTCAAGCTCTAAATCGGGGCATCCCTTTAGGGTTCCGATTTAGTGCTTTACGGCACCTCGACC |
| | CCAAAAAACTTGATTAGGGTGATGGTTCACGTAGTGGGCCATCGCCCTGATAGACGGTTTTTCGC |
| | CCTTTGACGTTGGAGTCCACGTTCTTTAATAGTGGACTCTTGTTCCAAACTGGAACAACACTCAA |
| | CCCTATCTCGGTCTATTCTTTTGATTTATAAGGGATTTTGGGGATTTCGGCCTATTGGTTAAAAA |
| | ATGAGCTGATTTAACAAAAATTTAACGCGAATTAATTCTGTGGAATGTGTGTCAGTTAGGGTGTG |
| | GAAAGTCCCCAGGCTCCCCAGGCAGGCAGAAGTATGCAAAGCATGCATCTCAATTAGTCAGCAAC |
| | CAGGTGTGGAAAGTCCCCAGGCTCCCCAGCAGGCAGAAGTATGCAAAGCATGCATCTCAATTAGT |
| | CAGCAACCATAGTCCCGCCCCTAACTCCGCCCATCCCGCCCCTAACTCCGCCCAGTTCCGCCCAT |
| | TCTCCGCCCCATGGCTGACTAATTTTTTTTATTTATGCAGAGGCCGAGGCCGCCTCTGCCTCTGA |
| | GCTATTCCAGAAGTAGTGAGGAGGCTTTTTTGGAGGCCTAGGCTTTTGCAAAAAGCTCCCGGGAG |
| | CTTGTATATCCATTTTCGGATCTGATCAAGAGACAGGATGAGGATCGTTTCGCATGATTGAACAA |
| | GATGGATTGCACGCAGGTTCTCCGGCCGCTTGGGTGGAGAGGCTATTCGGCTATGACTGGGCACA |
| | ACAGACAATCGGCTGCTCTGATGCCGCCGTGTTCCGGCTGTCAGCGCAGGGGCGCCCGGTTCTTT |
| | TTGTCAAGACCGACCTGTCCGGTGCCCTGAATGAACTGCAGGACGAGGCAGCGCGGCTATCGTGG |
| | CTGGCCACGACGGGCGTTCCTTGCGCAGCTGTGCTCGACGTTGTCACTGAAGCGGGAAGGGACTG |
| | GCTGCTATTGGGCGAAGTGCCGGGGCAGGATCTCCTGTCATCTCACCTTGCTCCTGCCGAGAAAG |
| | TATCCATCATGGCTGATGCAATGCGGCGGCTGCATACGCTTGATCCGGCTACCTGCCCATTCGAC |
| | CACCAAGCGAAACATCGCATCGAGCGAGCACGTACTCGGATGGAAGCCGGTCTTGTCGATCAGGA |
| | TGATCTGGACGAAGAGCATCAGGGGCTCGCGCCAGCCGAACTGTTCGCCAGGCTCAAGGCGCGCA |
| | TGCCCGACGGCGAGGATCTCGTCGTGACCCATGGCGATGCCTGCTTGCCGAATATCATGGTGGAA |
| | AATGGCCGCTTTTCTGGATTCATCGACTGTGGCCGGCTGGGTGTGGCGGACCGCTATCAGGACAT |
| | AGCGTTGGCTACCCGTGATATTGCTGAAGAGCTTGGCGGCGAATGGGCTGACCGCTTCCTCGTGC |
| | TTTACGGTATCGCCGCTCCCGATTCGCAGCGCATCGCCTTCTATCGCCTTCTTGACGAGTTCTTC |
| | TGAGCGGGACTCTGGGGTTCGCGAAATGACCGACCAAGCGACGCCCAACCTGCCATCACGAGATT |
| | TCGATTCCACCGCCGCCTTCTATGAAAGGTTGGGCTTCGGAATCGTTTTCCGGGACGCCGGCTGG |
| | ATGATCCTCCAGCGCGGGGATCTCATGCTGGAGTTCTTCGCCCACCCCAACTTGTTTATTGCAGC |
| | TTATAATGGTTACAAATAAAGCAATAGCATCACAAATTTCACAAATAAAGCATTTTTTTCACTGC |
| | ATTCTAGTTGTGGTTTGTCCAAACTCATCAATGTATCTTATCATGTCTGTATACCGTCGACCTCT |
| | AGCTAGAGCTTGGCGTAATCATGGTCATAGCTGTTTCCTGTGTGAAATTGTTATCCGCTCACAAT |
| | TCCACACAACATACGAGCCGGAAGCATAAAGTGTAAAGCCTGGGGTGCCTAATGAGTGAGCTAAC |
| | TCACATTAATTGCGTTGCGCTCACTGCCCGCTTTCCAGTCGGGAAACCTGTCGTGCCAGCTGCAT |
| | TAATGAATCGGCCAACGCGCGGGGAGAGGCGGTTTGCGTATTGGGCGCTCTTCCGCTTCCTCGCT |
| | CACTGACTCGCTGCGCTCGGTCGTTCGGCTGCGGCGAGCGGTATCAGCTCACTCAAAGGCGGTAA |
| | TACGGTTATCCACAGAATCAGGGGATAACGCAGGAAAGAACATGTGAGCAAAAGGCCAGCAAAAG |
| | GCCAGGAACCGTAAAAAGGCCGCGTTGCTGGCGTTTTTCCATAGGCTCCGCCCCCCTGACGAGCA |
| | TCACAAAAATCGACGCTCAAGTCAGAGGTGGCGAAACCCGACAGGACTATAAAGATACCAGGCGT |
| | TTCCCCCTGGAAGCTCCCTCGTGCGCTCTCCTGTTCCGACCCTGCCGCTTACCGGATACCTGTCC |
| | GCCTTTCTCCCTTCGGGAAGCGTGGCGCTTTCTCAATGCTCACGCTGTAGGTATCTCAGTTCGGT |
| | GTAGGTCGTTCGCTCCAAGCTGGGCTGTGTGCACGAACCCCCCGTTCAGCCCGACCGCTGCGCCT |
| | TATCCGGTAACTATCGTCTTGAGTCCAACCCGGTAAGACACGACTTATCGCCACTGGCAGCAGCC |
| | ACTGGTAACAGGATTAGCAGAGCGAGGTATGTAGGCGGTGCTACAGAGTTCTTGAAGTGGTGGCC |
| | TAACTACGGCTACACTAGAAGGACAGTATTTGGTATCTGCGCTCTGCTGAAGCCAGTTACCTTCG |
| | GAAAAAGAGTTGGTAGCTCTTGATCCGGCAAACAAACCACCGCTGGTAGCGGTGGTTTTTTTGTT |
| | TGCAAGCAGCAGATTACGCGCAGAAAAAAAGGATCTCAAGAAGATCCTTTGATCTTTTCTACGGG |
| | GTCTGACGCTCAGTGGAACGAAAACTCACGTTAAGGGATTTTGGTCATGAGATTATCAAAAAGGA |
| | TCTTCACCTAGATCCTTTTAAATTAAAAATGAAGTTTTAAATCAATCTAAAGTATATATGAGTAA |
| | ACTTGGTCTGACAGTTACCAATGCTTAATCAGTGAGGCACCTATCTCAGCGATCTGTCTATTTCG |
| | TTCATCCATAGTTGCCTGACTCCCCGTCGTGTAGATAACTACGATACGGGAGGGCTTACCATCTG |
| | GCCCCAGTGCTGCAATGATACCGCGAGACCCACGCTCACCGGCTCCAGATTTATCAGCAATAAAC |
| | CAGCCAGCCGGAAGGGCCGAGCGCAGAAGTGGTCCTGCAACTTTATCCGCCTCCATCCAGTCTAT |
| | TAATTGTTGCCGGGAAGCTAGAGTAAGTAGTTCGCCAGTTAATAGTTTGCGCAACGTTGTTGCCA |
| | TTGCTACAGGCATCGTGGTGTCACGCTCGTCGTTTGGTATGGCTTCATTCAGCTCCGGTTCCCAA |
| | CGATCAAGGCGAGTTACATGATCCCCCATGTTGTGCAAAAAAGCGGTTAGCTCCTTCGGTCCTCC |
| | GATCGTTGTCAGAAGTAAGTTGGCCGCAGTGTTATCACTCATGGTTATGGCAGCACTGCATAATT |
| | CTCTTACTGTCATGCCATCCGTAAGATGCTTTTCTGTGACTGGTGAGTACTCAACCAAGTCATTC |
| | TGAGAATAGTGTATGCGGCGACCGAGTTGCTCTTGCCCGGCGTCAATACGGGATAATACCGCGCC |
| | ACATAGCAGAACTTTAAAAGTGCTCATCATTGGAAAACGTTCTTCGGGGCGAAAACTCTCAAGGA |
| | TCTTACCGCTGTTGAGATCCAGTTCGATGTAACCCACTCGTGCACCCAACTGATCTTCAGCATCT |
| | TTTACTTTCACCAGCGTTTCTGGGTGAGCAAAAACAGGAAGGCAAAATGCCGCAAAAAAGGGAAT |
| | AAGGGCGACACGGAAATGTTGAATACTCATACTCTTCCTTTTTCAATATTATTGAAGCATTTATC |
| | AGGGTTATTGTCTCATGAGCGGATACATATTTGAATGTATTTAGAAAAATAAACAAATAGGGGTT |
| | CCGCGCACATTTCCCCGAAAAGTGCCACCTGACGTC |
| BLING | MGVKVLFALICIAVAEAKPTENNEDFNIVAVASNFATTDLDADRGKLPGKKLPLEVLKELEANAR |
| 0.1 | KAGCTRGCLICLSHIKCTPKMKKFIPGRCHTYEGDKESAQGGGGTSTLDKIAKNGVIVVGHRESS |
| AA | VPFSYYDNQQKVVGYSQDYSNAIVEAVKKKLNKPDLQVKLIPITTQNRIPLLQNGTFDFECGSTT |
| (SEQ | NNVERQKQAAFSDTIFVVGTRLLTKKGGDIKDFANLKDKAVVVTSGTTSEVLLNKLNEEQKMNMR |
| ID | IISAKDHGDSFRTLESGRAVAFMMDDALLAGERAKAKKPDNWEIVGKPQSQEAYGCMLRKDDPQF |
| NO: | KKLMDDTIAQVQTSGEAEKWFDKWFKNPIPPKNLNMNFELSDEMKALFKEPNDGSGGGGGIGEAI |
| 2) | VDIPEIPGFKDLEPLEQFIAQVDLCVDCTTGCLKGLANVQCSDLLKKWLPQRCATFASKIQGQVD |
| | KIKGAGGDLEAVGQDTQEVIVVPHSLPFKVVVISAILALVVLTIISLIILIMLWQKKPR |
| BLING | GACGGATCGGGAGATCTCCCGATCCCCTATGGTCGACTCTCAGTACAATCTGCTCTGATGCCGCA |
| 0.2 | TAGTTAAGCCAGTATCTGCTCCCTGCTTGTGTGTTGGAGGTCGCTGAGTAGTGCGCGAGCAAAAT |
| DNA | TTAAGCTACAACAAGGCAAGGCTTGACCGACAATTGCATGAAGAATCTGCTTAGGGTTAGGCGTT |
| (SEQ | TTGCGCTGCTTCGCGATGTACGGGCCAGATATACGCGTTGACATTGATTATTGACTAGTTATTAA |
| ID | TAGTAATCAATTACGGGGTCATTAGTTCATAGCCCATATATGGAGTTCCGCGTTACATAACTTAC |
| NO: | GGTAAATGGCCCGCCTGGCTGACCGCCCAACGACCCCCGCCCATTGACGTCAATAATGACGTATG |
| 3) | TTCCCATAGTAACGCCAATAGGGACTTTCCATTGACGTCAATGGGTGGACTATTTACGGTAAACT |
| | GCCCACTTGGCAGTACATCAAGTGTATCATATGCCAAGTACGCCCCCTATTGACGTCAATGACGG |
| | TAAATGGCCCGCCTGGCATTATGCCCAGTACATGACCTTATGGGACTTTCCTACTTGGCAGTACA |
| | TCTACGTATTAGTCATCGCTATTACCATGGTGATGCGGTTTTGGCAGTACATCAATGGGCGTGGA |
| | TAGCGGTTTGACTCACGGGGATTTCCAAGTCTCCACCCCATTGACGTCAATGGGAGTTTGTTTTG |
| | GCACCAAAATCAACGGGACTTTCCAAAATGTCGTAACAACTCCGCCCCATTGACGCAAATGGGCG |
| | GTAGGCGTGTACGGTGGGAGGTCTATATAAGCAGAGCTCTCTGGCTAACTAGAGAACCCACTGCT |
| | TACTGGCTTATCGAAATTAATACGACTCACTATAGGGAGACCCAAGCTGGCTAGTTAAGCTTGCC |
| | ATGGAGACAGACACACTCCTGCTATGGGTACTGCTGCTCTGGGTTCCAGGTTCCACTGGTGACAT |
| | GGTCTTCACACTCGAAGATTTCGTTGGGGACTGGCGACAGACAGCCGGCTACAACCTGGACCAAG |
| | TCCTTGAACAGGGAGGTGTGTCCAGTTTGTTTCAGAATCTCGGGGTGTCCGTAACTCCGATCCAA |
| | AGGATTGTCCTGAGCGGTGAAAATGGGCTGAAGATCGACATCCATGTCATCATCCCGTATGAAGG |
| | TGGAGGAGGCGGTACCAGTACTCTGGACAAAATCGCCAAAAACGGTGTGATTGTCGTCGGTCACC |
| | GTGAATCTTCAGTGCCTTTCTCTTATTACGACAATCAGCAAAAAGTGGTGGGTTACTCGCAGGAT |
| | TACTCCAACGCCATTGTTGAAGCAGTGAAAAAGAAACTCAACAAACCGGACTTGCAGGTAAAACT |
| | GATTCCGATTACAACACAAAACCGTATTCCACTGCTGCAAAACGGCACTTTCGATTTTGAATGTG |
| | GTTCTACCACCAACAACGTCGAACGCCAAAAACAGGCGGCTTTCTCTGACACTATTTTCGTGGTC |
| | GGTACGCGCCTGTTGACCAAAAAGGGTGGCGATATCAAAGATTTTGCCAACCTGAAAGACAAAGC |
| | CGTAGTCGTCACTTCCGGCACTACCTCTGAAGTTTTGCTCAACAAACTGAATGAAGAGCAAAAAA |
| | TGAATATGCGCATCATCAGCGCCAAAGATCACGGTGACTCTTTCCGCACCCTGGAAAGCGGTCGT |
| | GCCGTTGCCTTTATGATGGATGACGCTCTGCTGGCCGGTGAACGTGCGAAAGCGAAGAAACCAGA |
| | CAACTGGGAAATCGTCGGCAAGCCGCAGTCTCAGGAGGCCTACGGTTGTATGTTGCGTAAAGATG |
| | ATCCGCAGTTCAAAAAGCTGATGGATGACACCATCGCTCAGGTGCAGACCTCCGGTGAAGCGGAA |
| | AAATGGTTTGATAAGTGGTTCAAAAATCCAATTCCGCCGAAAAACCTGAACATGAATTTCGAACT |
| | GTCAGACGAAATGAAAGCACTGTTCAAAGAACCGAATGACGGATCCGGAGGCGGCCTGAGCGGCG |
| | ACCAAATGGGCCAGATCGAAAAAATTTTTAAGGTGGTGTACCCTGTGGATGATCATCACTTTAAG |
| | GTGATCCTGCACTATGGCACACTGGTAATCGACGGGGTTACGCCGAACATGATCGACTATTTCGG |
| | ACGGCCGTATGAAGGCATCGCCGTGTTCGACGGCAAAAAGATCACTGTAACAGGGACCCTGTGGA |
| | ACGGCAACAAAATTATCGACGAGCGCCTGATCAACCCCGACGGCTCCCTGCTGTTCCGAGTAACC |
| | ATCAACGGAGTGACCGGCTGGCGGCTGTGCGAACGCATTCTGGCGCTCGAGGCTGTGGGCCAGGA |
| | CACGCAGGAGGTCATCGTGGTGCCACACTCCTTGCCCTTTAAGGTGGTGGTGATCTCAGCCATCC |
| | TGGCCCTGGTGGTGCTCACCATCATCTCCCTTATCATCCTCATCATGCTTTGGCAGAAGAAGCCA |
| | CGTTAGTAACATATGCTCTAGAGGGCCCGCGGTTCGAAGGTAAGCCTATCCCTAACCCTCTCCTC |
| | GGTCTCGATTCTACGCGTACCGGTCATCATCACCATCACCATTGAGTTTAAACCCGCTGATCAGC |
| | CTCGACTGTGCCTTCTAGTTGCCAGCCATCTGTTGTTTGCCCCTCCCCCGTGCCTTCCTTGACCC |
| | TGGAAGGTGCCACTCCCACTGTCCTTTCCTAATAAAATGAGGAAATTGCATCGCATTGTCTGAGT |
| | AGGTGTCATTCTATTCTGGGGGGTGGGGTGGGGCAGGACAGCAAGGGGGAGGATTGGGAAGACAA |
| | TAGCAGGCATGCTGGGGATGCGGTGGGCTCTATGGCTTCTGAGGCGGAAAGAACCAGCTGGGGCT |
| | CTAGGGGGTATCCCCACGCGCCCTGTAGCGGCGCATTAAGCGCGGCGGGTGTGGTGGTTACGCGC |
| | AGCGTGACCGCTACACTTGCCAGCGCCCTAGCGCCCGCTCCTTTCGCTTTCTTCCCTTCCTTTCT |
| | CGCCACGTTCGCCGGCTTTCCCCGTCAAGCTCTAAATCGGGGCATCCCTTTAGGGTTCCGATTTA |
| | GTGCTTTACGGCACCTCGACCCCAAAAAACTTGATTAGGGTGATGGTTCACGTAGTGGGCCATCG |
| | CCCTGATAGACGGTTTTTCGCCCTTTGACGTTGGAGTCCACGTTCTTTAATAGTGGACTCTTGTT |
| | CCAAACTGGAACAACACTCAACCCTATCTCGGTCTATTCTTTTGATTTATAAGGGATTTTGGGGA |
| | TTTCGGCCTATTGGTTAAAAAATGAGCTGATTTAACAAAAATTTAACGCGAATTAATTCTGTGGA |
| | ATGTGTGTCAGTTAGGGTGTGGAAAGTCCCCAGGCTCCCCAGGCAGGCAGAAGTATGCAAAGCAT |
| | GCATCTCAATTAGTCAGCAACCAGGTGTGGAAAGTCCCCAGGCTCCCCAGCAGGCAGAAGTATGC |
| | AAAGCATGCATCTCAATTAGTCAGCAACCATAGTCCCGCCCCTAACTCCGCCCATCCCGCCCCTA |
| | ACTCCGCCCAGTTCCGCCCATTCTCCGCCCCATGGCTGACTAATTTTTTTTATTTATGCAGAGGC |
| | CGAGGCCGCCTCTGCCTCTGAGCTATTCCAGAAGTAGTGAGGAGGCTTTTTTGGAGGCCTAGGCT |
| | TTTGCAAAAAGCTCCCGGGAGCTTGTATATCCATTTTCGGATCTGATCAAGAGACAGGATGAGGA |
| | TCGTTTCGCATGATTGAACAAGATGGATTGCACGCAGGTTCTCCGGCCGCTTGGGTGGAGAGGCT |
| | ATTCGGCTATGACTGGGCACAACAGACAATCGGCTGCTCTGATGCCGCCGTGTTCCGGCTGTCAG |
| | CGCAGGGGCGCCCGGTTCTTTTTGTCAAGACCGACCTGTCCGGTGCCCTGAATGAACTGCAGGAC |
| | GAGGCAGCGCGGCTATCGTGGCTGGCCACGACGGGCGTTCCTTGCGCAGCTGTGCTCGACGTTGT |
| | CACTGAAGCGGGAAGGGACTGGCTGCTATTGGGCGAAGTGCCGGGGCAGGATCTCCTGTCATCTC |
| | ACCTTGCTCCTGCCGAGAAAGTATCCATCATGGCTGATGCAATGCGGCGGCTGCATACGCTTGAT |
| | CCGGCTACCTGCCCATTCGACCACCAAGCGAAACATCGCATCGAGCGAGCACGTACTCGGATGGA |
| | AGCCGGTCTTGTCGATCAGGATGATCTGGACGAAGAGCATCAGGGGCTCGCGCCAGCCGAACTGT |
| | TCGCCAGGCTCAAGGCGCGCATGCCCGACGGCGAGGATCTCGTCGTGACCCATGGCGATGCCTGC |
| | TTGCCGAATATCATGGTGGAAAATGGCCGCTTTTCTGGATTCATCGACTGTGGCCGGCTGGGTGT |
| | GGCGGACCGCTATCAGGACATAGCGTTGGCTACCCGTGATATTGCTGAAGAGCTTGGCGGCGAAT |
| | GGGCTGACCGCTTCCTCGTGCTTTACGGTATCGCCGCTCCCGATTCGCAGCGCATCGCCTTCTAT |
| | CGCCTTCTTGACGAGTTCTTCTGAGCGGGACTCTGGGGTTCGCGAAATGACCGACCAAGCGACGC |
| | CCAACCTGCCATCACGAGATTTCGATTCCACCGCCGCCTTCTATGAAAGGTTGGGCTTCGGAATC |
| | GTTTTCCGGGACGCCGGCTGGATGATCCTCCAGCGCGGGGATCTCATGCTGGAGTTCTTCGCCCA |
| | CCCCAACTTGTTTATTGCAGCTTATAATGGTTACAAATAAAGCAATAGCATCACAAATTTCACAA |
| | ATAAAGCATTTTTTTCACTGCATTCTAGTTGTGGTTTGTCCAAACTCATCAATGTATCTTATCAT |
| | GTCTGTATACCGTCGACCTCTAGCTAGAGCTTGGCGTAATCATGGTCATAGCTGTTTCCTGTGTG |
| | AAATTGTTATCCGCTCACAATTCCACACAACATACGAGCCGGAAGCATAAAGTGTAAAGCCTGGG |
| | GTGCCTAATGAGTGAGCTAACTCACATTAATTGCGTTGCGCTCACTGCCCGCTTTCCAGTCGGGA |
| | AACCTGTCGTGCCAGCTGCATTAATGAATCGGCCAACGCGCGGGGAGAGGCGGTTTGCGTATTGG |
| | GCGCTCTTCCGCTTCCTCGCTCACTGACTCGCTGCGCTCGGTCGTTCGGCTGCGGCGAGCGGTAT |
| | CAGCTCACTCAAAGGCGGTAATACGGTTATCCACAGAATCAGGGGATAACGCAGGAAAGAACATG |
| | TGAGCAAAAGGCCAGCAAAAGGCCAGGAACCGTAAAAAGGCCGCGTTGCTGGCGTTTTTCCATAG |
| | GCTCCGCCCCCCTGACGAGCATCACAAAAATCGACGCTCAAGTCAGAGGTGGCGAAACCCGACAG |
| | GACTATAAAGATACCAGGCGTTTCCCCCTGGAAGCTCCCTCGTGCGCTCTCCTGTTCCGACCCTG |
| | CCGCTTACCGGATACCTGTCCGCCTTTCTCCCTTCGGGAAGCGTGGCGCTTTCTCAATGCTCACG |
| | CTGTAGGTATCTCAGTTCGGTGTAGGTCGTTCGCTCCAAGCTGGGCTGTGTGCACGAACCCCCCG |
| | TTCAGCCCGACCGCTGCGCCTTATCCGGTAACTATCGTCTTGAGTCCAACCCGGTAAGACACGAC |
| | TTATCGCCACTGGCAGCAGCCACTGGTAACAGGATTAGCAGAGCGAGGTATGTAGGCGGTGCTAC |
| | AGAGTTCTTGAAGTGGTGGCCTAACTACGGCTACACTAGAAGGACAGTATTTGGTATCTGCGCTC |
| | TGCTGAAGCCAGTTACCTTCGGAAAAAGAGTTGGTAGCTCTTGATCCGGCAAACAAACCACCGCT |
| | GGTAGCGGTGGTTTTTTTGTTTGCAAGCAGCAGATTACGCGCAGAAAAAAAGGATCTCAAGAAGA |
| | TCCTTTGATCTTTTCTACGGGGTCTGACGCTCAGTGGAACGAAAACTCACGTTAAGGGATTTTGG |
| | TCATGAGATTATCAAAAAGGATCTTCACCTAGATCCTTTTAAATTAAAAATGAAGTTTTAAATCA |
| | ATCTAAAGTATATATGAGTAAACTTGGTCTGACAGTTACCAATGCTTAATCAGTGAGGCACCTAT |
| | CTCAGCGATCTGTCTATTTCGTTCATCCATAGTTGCCTGACTCCCCGTCGTGTAGATAACTACGA |
| | TACGGGAGGGCTTACCATCTGGCCCCAGTGCTGCAATGATACCGCGAGACCCACGCTCACCGGCT |
| | CCAGATTTATCAGCAATAAACCAGCCAGCCGGAAGGGCCGAGCGCAGAAGTGGTCCTGCAACTTT |
| | ATCCGCCTCCATCCAGTCTATTAATTGTTGCCGGGAAGCTAGAGTAAGTAGTTCGCCAGTTAATA |
| | GTTTGCGCAACGTTGTTGCCATTGCTACAGGCATCGTGGTGTCACGCTCGTCGTTTGGTATGGCT |
| | TCATTCAGCTCCGGTTCCCAACGATCAAGGCGAGTTACATGATCCCCCATGTTGTGCAAAAAAGC |
| | GGTTAGCTCCTTCGGTCCTCCGATCGTTGTCAGAAGTAAGTTGGCCGCAGTGTTATCACTCATGG |
| | TTATGGCAGCACTGCATAATTCTCTTACTGTCATGCCATCCGTAAGATGCTTTTCTGTGACTGGT |
| | GAGTACTCAACCAAGTCATTCTGAGAATAGTGTATGCGGCGACCGAGTTGCTCTTGCCCGGCGTC |
| | AATACGGGATAATACCGCGCCACATAGCAGAACTTTAAAAGTGCTCATCATTGGAAAACGTTCTT |
| | CGGGGCGAAAACTCTCAAGGATCTTACCGCTGTTGAGATCCAGTTCGATGTAACCCACTCGTGCA |
| | CCCAACTGATCTTCAGCATCTTTTACTTTCACCAGCGTTTCTGGGTGAGCAAAAACAGGAAGGCA |
| | AAATGCCGCAAAAAAGGGAATAAGGGCGACACGGAAATGTTGAATACTCATACTCTTCCTTTTTC |
| | AATATTATTGAAGCATTTATCAGGGTTATTGTCTCATGAGCGGATACATATTTGAATGTATTTAG |
| | AAAAATAAACAAATAGGGGTTCCGCGCACATTTCCCCGAAAAGTGCCACCTGACGTC |
| BLING | METDTLLLWVLLLWVPGSTGDMVFTLEDFVGDWRQTAGYNLDQVLEQGGVSSLFQNLGVSVTPIQ |
| 0.2 | RIVLSGENGLKIDIHVIIPYEGGGGGTSTLDKIAKNGVIVVGHRESSVPFSYYDNQQKVVGYSQD |
| AA | YSNAIVEAVKKKLNKPDLQVKLIPITTQNRIPLLQNGTFDFECGSTTNNVERQKQAAFSDTIFVV |
| (SEQ | GTRLLTKKGGDIKDFANLKDKAVVVTSGTTSEVLLNKLNEEQKMNMRIISAKDHGDSFRTLESGR |
| ID | AVAFMMDDALLAGERAKAKKPDNWEIVGKPQSQEAYGCMLRKDDPQFKKLMDDTIAQVQTSGEAE |
| NO: | KWFDKWFKNPIPPKNLNMNFELSDEMKALFKEPNDGSGGGLSGDQMGQIEKIFKVVYPVDDHHFK |
| 4) | VILHYGTLVIDGVTPNMIDYFGRPYEGIAVFDGKKITVTGTLWNGNKIIDERLINPDGSLLFRVT |
| | INGVTGWRLCERILALEAVGQDTQEVIVVPHSLPFKVVVISAILALVVLTIISLIILIMLWQKKP |
| | R |
| BLING | GACGGATCGGGAGATCTCCCGATCCCCTATGGTCGACTCTCAGTACAATCTGCTCTGATGCCGCA |
| 0.3 | TAGTTAAGCCAGTATCTGCTCCCTGCTTGTGTGTTGGAGGTCGCTGAGTAGTGCGCGAGCAAAAT |
| DNA | TTAAGCTACAACAAGGCAAGGCTTGACCGACAATTGCATGAAGAATCTGCTTAGGGTTAGGCGTT |
| (SEQ | TTGCGCTGCTTCGCGATGTACGGGCCAGATATACGCGTTGACATTGATTATTGACTAGTTATTAA |
| ID | TAGTAATCAATTACGGGGTCATTAGTTCATAGCCCATATATGGAGTTCCGCGTTACATAACTTAC |
| NO: | GGTAAATGGCCCGCCTGGCTGACCGCCCAACGACCCCCGCCCATTGACGTCAATAATGACGTATG |
| 5) | TTCCCATAGTAACGCCAATAGGGACTTTCCATTGACGTCAATGGGTGGACTATTTACGGTAAACT |
| | GCCCACTTGGCAGTACATCAAGTGTATCATATGCCAAGTACGCCCCCTATTGACGTCAATGACGG |
| | TAAATGGCCCGCCTGGCATTATGCCCAGTACATGACCTTATGGGACTTTCCTACTTGGCAGTACA |
| | TCTACGTATTAGTCATCGCTATTACCATGGTGATGCGGTTTTGGCAGTACATCAATGGGCGTGGA |
| | TAGCGGTTTGACTCACGGGGATTTCCAAGTCTCCACCCCATTGACGTCAATGGGAGTTTGTTTTG |
| | GCACCAAAATCAACGGGACTTTCCAAAATGTCGTAACAACTCCGCCCCATTGACGCAAATGGGCG |
| | GTAGGCGTGTACGGTGGGAGGTCTATATAAGCAGAGCTCTCTGGCTAACTAGAGAACCCACTGCT |
| | TACTGGCTTATCGAAATTAATACGACTCACTATAGGGAGACCCAAGCTGGCTAGTTAAGCTTGCC |
| | ATGGAGACAGACACACTCCTGCTATGGGTACTGCTGCTCTGGGTTCCAGGTTCCACTGGTGACAT |
| | GGTCTTCACACTCGAAGATTTCGTTGGGGACTGGCGACAGACAGCCGGCTACAACCTGGACCAAG |
| | TCCTTGAACAGGGAGGTGTGTCCAGTTTGTTTCAGAATCTCGGGGTGTCCGTAACTCCGATCCAA |
| | AGGATTGTCCTGAGCGGTGAAAATGGGCTGAAGATCGACATCCATGTCATCATCCCGTATGAAGG |
| | TCTGAGCGGCGACCAAATGGGCCAGATCGAAAAAATTTTTAAGGTGGTGTACCCTGTGGATGATC |
| | ATCACTTTAAGGTGATCCTGCACTATGGCACACTGGTAATCGACGGGGTTACGCCGAACATGATC |
| | GACTATTTCGGACGGCCGTATGAAGGCATCGCCGTGTTCGACGGCAAAAAGATCACTGTAACAGG |
| | GACCCTGTGGAACGGCAACAAAATTATCGACGAGCGCCTGATCAACCCCGACGGCTCCCTGCTGT |
| | TCCGAGTAACCATCAACGGAGGAGGAGGCGGTACCAGTACTCTGGACAAAATCGCCAAAAACGGT |
| | GTGATTGTCGTCGGTCACCGTGAATCTTCAGTGCCTTTCTCTTATTACGACAATCAGCAAAAAGT |
| | GGTGGGTTACTCGCAGGATTACTCCAACGCCATTGTTGAAGCAGTGAAAAAGAAACTCAACAAAC |
| | CGGACTTGCAGGTAAAACTGATTCCGATTACAACACAAAACCGTATTCCACTGCTGCAAAACGGC |
| | ACTTTCGATTTTGAATGTGGTTCTACCACCAACAACGTCGAACGCCAAAAACAGGCGGCTTTCTC |
| | TGACACTATTTTCGTGGTCGGTACGCGCCTGTTGACCAAAAAGGGTGGCGATATCAAAGATTTTG |
| | CCAACCTGAAAGACAAAGCCGTAGTCGTCACTTCCGGCACTACCTCTGAAGTTTTGCTCAACAAA |
| | CTGAATGAAGAGCAAAAAATGAATATGCGCATCATCAGCGCCAAAGATCACGGTGACTCTTTCCG |
| | CACCCTGGAAAGCGGTCGTGCCGTTGCCTTTATGATGGATGACGCTCTGCTGGCCGGTGAACGTG |
| | CGAAAGCGAAGAAACCAGACAACTGGGAAATCGTCGGCAAGCCGCAGTCTCAGGAGGCCTACGGT |
| | TGTATGTTGCGTAAAGATGATCCGCAGTTCAAAAAGCTGATGGATGACACCATCGCTCAGGTGCA |
| | GACCTCCGGTGAAGCGGAAAAATGGTTTGATAAGTGGTTCAAAAATCCAATTCCGCCGAAAAACC |
| | TGAACATGAATTTCGAACTGTCAGACGAAATGAAAGCACTGTTCAAAGAACCGAATGACGGATCC |
| | GGAGGCGGCGTGACCGGCTGGCGGCTGTGCGAACGCATTCTGCTCGAGGCTGTGGGCCAGGACAC |
| | GCAGGAGGTCATCGTGGTGCCACACTCCTTGCCCTTTAAGGTGGTGGTGATCTCAGCCATCCTGG |
| | CCCTGGTGGTGCTCACCATCATCTCCCTTATCATCCTCATCATGCTTTGGCAGAAGAAGCCACGT |
| | TAGTAACATATGCTCTAGAGGGCCCGCGGTTCGAAGGTAAGCCTATCCCTAACCCTCTCCTCGGT |
| | CTCGATTCTACGCGTACCGGTCATCATCACCATCACCATTGAGTTTAAACCCGCTGATCAGCCTC |
| | GACTGTGCCTTCTAGTTGCCAGCCATCTGTTGTTTGCCCCTCCCCCGTGCCTTCCTTGACCCTGG |
| | AAGGTGCCACTCCCACTGTCCTTTCCTAATAAAATGAGGAAATTGCATCGCATTGTCTGAGTAGG |
| | TGTCATTCTATTCTGGGGGGTGGGGTGGGGCAGGACAGCAAGGGGGAGGATTGGGAAGACAATAG |
| | CAGGCATGCTGGGGATGCGGTGGGCTCTATGGCTTCTGAGGCGGAAAGAACCAGCTGGGGCTCTA |
| | GGGGGTATCCCCACGCGCCCTGTAGCGGCGCATTAAGCGCGGCGGGTGTGGTGGTTACGCGCAGC |
| | GTGACCGCTACACTTGCCAGCGCCCTAGCGCCCGCTCCTTTCGCTTTCTTCCCTTCCTTTCTCGC |
| | CACGTTCGCCGGCTTTCCCCGTCAAGCTCTAAATCGGGGCATCCCTTTAGGGTTCCGATTTAGTG |
| | CTTTACGGCACCTCGACCCCAAAAAACTTGATTAGGGTGATGGTTCACGTAGTGGGCCATCGCCC |
| | TGATAGACGGTTTTTCGCCCTTTGACGTTGGAGTCCACGTTCTTTAATAGTGGACTCTTGTTCCA |
| | AACTGGAACAACACTCAACCCTATCTCGGTCTATTCTTTTGATTTATAAGGGATTTTGGGGATTT |
| | CGGCCTATTGGTTAAAAAATGAGCTGATTTAACAAAAATTTAACGCGAATTAATTCTGTGGAATG |
| | TGTGTCAGTTAGGGTGTGGAAAGTCCCCAGGCTCCCCAGGCAGGCAGAAGTATGCAAAGCATGCA |
| | TCTCAATTAGTCAGCAACCAGGTGTGGAAAGTCCCCAGGCTCCCCAGCAGGCAGAAGTATGCAAA |
| | GCATGCATCTCAATTAGTCAGCAACCATAGTCCCGCCCCTAACTCCGCCCATCCCGCCCCTAACT |
| | CCGCCCAGTTCCGCCCATTCTCCGCCCCATGGCTGACTAATTTTTTTTATTTATGCAGAGGCCGA |
| | GGCCGCCTCTGCCTCTGAGCTATTCCAGAAGTAGTGAGGAGGCTTTTTTGGAGGCCTAGGCTTTT |
| | GCAAAAAGCTCCCGGGAGCTTGTATATCCATTTTCGGATCTGATCAAGAGACAGGATGAGGATCG |
| | TTTCGCATGATTGAACAAGATGGATTGCACGCAGGTTCTCCGGCCGCTTGGGTGGAGAGGCTATT |
| | CGGCTATGACTGGGCACAACAGACAATCGGCTGCTCTGATGCCGCCGTGTTCCGGCTGTCAGCGC |
| | AGGGGCGCCCGGTTCTTTTTGTCAAGACCGACCTGTCCGGTGCCCTGAATGAACTGCAGGACGAG |
| | GCAGCGCGGCTATCGTGGCTGGCCACGACGGGCGTTCCTTGCGCAGCTGTGCTCGACGTTGTCAC |
| | TGAAGCGGGAAGGGACTGGCTGCTATTGGGCGAAGTGCCGGGGCAGGATCTCCTGTCATCTCACC |
| | TTGCTCCTGCCGAGAAAGTATCCATCATGGCTGATGCAATGCGGCGGCTGCATACGCTTGATCCG |
| | GCTACCTGCCCATTCGACCACCAAGCGAAACATCGCATCGAGCGAGCACGTACTCGGATGGAAGC |
| | CGGTCTTGTCGATCAGGATGATCTGGACGAAGAGCATCAGGGGCTCGCGCCAGCCGAACTGTTCG |
| | CCAGGCTCAAGGCGCGCATGCCCGACGGCGAGGATCTCGTCGTGACCCATGGCGATGCCTGCTTG |
| | CCGAATATCATGGTGGAAAATGGCCGCTTTTCTGGATTCATCGACTGTGGCCGGCTGGGTGTGGC |
| | GGACCGCTATCAGGACATAGCGTTGGCTACCCGTGATATTGCTGAAGAGCTTGGCGGCGAATGGG |
| | CTGACCGCTTCCTCGTGCTTTACGGTATCGCCGCTCCCGATTCGCAGCGCATCGCCTTCTATCGC |
| | CTTCTTGACGAGTTCTTCTGAGCGGGACTCTGGGGTTCGCGAAATGACCGACCAAGCGACGCCCA |
| | ACCTGCCATCACGAGATTTCGATTCCACCGCCGCCTTCTATGAAAGGTTGGGCTTCGGAATCGTT |
| | TTCCGGGACGCCGGCTGGATGATCCTCCAGCGCGGGGATCTCATGCTGGAGTTCTTCGCCCACCC |
| | CAACTTGTTTATTGCAGCTTATAATGGTTACAAATAAAGCAATAGCATCACAAATTTCACAAATA |
| | AAGCATTTTTTTCACTGCATTCTAGTTGTGGTTTGTCCAAACTCATCAATGTATCTTATCATGTC |
| | TGTATACCGTCGACCTCTAGCTAGAGCTTGGCGTAATCATGGTCATAGCTGTTTCCTGTGTGAAA |
| | TTGTTATCCGCTCACAATTCCACACAACATACGAGCCGGAAGCATAAAGTGTAAAGCCTGGGGTG |
| | CCTAATGAGTGAGCTAACTCACATTAATTGCGTTGCGCTCACTGCCCGCTTTCCAGTCGGGAAAC |
| | CTGTCGTGCCAGCTGCATTAATGAATCGGCCAACGCGCGGGGAGAGGCGGTTTGCGTATTGGGCG |
| | CTCTTCCGCTTCCTCGCTCACTGACTCGCTGCGCTCGGTCGTTCGGCTGCGGCGAGCGGTATCAG |
| | CTCACTCAAAGGCGGTAATACGGTTATCCACAGAATCAGGGGATAACGCAGGAAAGAACATGTGA |
| | GCAAAAGGCCAGCAAAAGGCCAGGAACCGTAAAAAGGCCGCGTTGCTGGCGTTTTTCCATAGGCT |
| | CCGCCCCCCTGACGAGCATCACAAAAATCGACGCTCAAGTCAGAGGTGGCGAAACCCGACAGGAC |
| | TATAAAGATACCAGGCGTTTCCCCCTGGAAGCTCCCTCGTGCGCTCTCCTGTTCCGACCCTGCCG |
| | CTTACCGGATACCTGTCCGCCTTTCTCCCTTCGGGAAGCGTGGCGCTTTCTCAATGCTCACGCTG |
| | TAGGTATCTCAGTTCGGTGTAGGTCGTTCGCTCCAAGCTGGGCTGTGTGCACGAACCCCCCGTTC |
| | AGCCCGACCGCTGCGCCTTATCCGGTAACTATCGTCTTGAGTCCAACCCGGTAAGACACGACTTA |
| | TCGCCACTGGCAGCAGCCACTGGTAACAGGATTAGCAGAGCGAGGTATGTAGGCGGTGCTACAGA |
| | GTTCTTGAAGTGGTGGCCTAACTACGGCTACACTAGAAGGACAGTATTTGGTATCTGCGCTCTGC |
| | TGAAGCCAGTTACCTTCGGAAAAAGAGTTGGTAGCTCTTGATCCGGCAAACAAACCACCGCTGGT |
| | AGCGGTGGTTTTTTTGTTTGCAAGCAGCAGATTACGCGCAGAAAAAAAGGATCTCAAGAAGATCC |
| | TTTGATCTTTTCTACGGGGTCTGACGCTCAGTGGAACGAAAACTCACGTTAAGGGATTTTGGTCA |
| | TGAGATTATCAAAAAGGATCTTCACCTAGATCCTTTTAAATTAAAAATGAAGTTTTAAATCAATC |
| | TAAAGTATATATGAGTAAACTTGGTCTGACAGTTACCAATGCTTAATCAGTGAGGCACCTATCTC |
| | AGCGATCTGTCTATTTCGTTCATCCATAGTTGCCTGACTCCCCGTCGTGTAGATAACTACGATAC |
| | GGGAGGGCTTACCATCTGGCCCCAGTGCTGCAATGATACCGCGAGACCCACGCTCACCGGCTCCA |
| | GATTTATCAGCAATAAACCAGCCAGCCGGAAGGGCCGAGCGCAGAAGTGGTCCTGCAACTTTATC |
| | CGCCTCCATCCAGTCTATTAATTGTTGCCGGGAAGCTAGAGTAAGTAGTTCGCCAGTTAATAGTT |
| | TGCGCAACGTTGTTGCCATTGCTACAGGCATCGTGGTGTCACGCTCGTCGTTTGGTATGGCTTCA |
| | TTCAGCTCCGGTTCCCAACGATCAAGGCGAGTTACATGATCCCCCATGTTGTGCAAAAAAGCGGT |
| | TAGCTCCTTCGGTCCTCCGATCGTTGTCAGAAGTAAGTTGGCCGCAGTGTTATCACTCATGGTTA |
| | TGGCAGCACTGCATAATTCTCTTACTGTCATGCCATCCGTAAGATGCTTTTCTGTGACTGGTGAG |
| | TACTCAACCAAGTCATTCTGAGAATAGTGTATGCGGCGACCGAGTTGCTCTTGCCCGGCGTCAAT |
| | ACGGGATAATACCGCGCCACATAGCAGAACTTTAAAAGTGCTCATCATTGGAAAACGTTCTTCGG |
| | GGCGAAAACTCTCAAGGATCTTACCGCTGTTGAGATCCAGTTCGATGTAACCCACTCGTGCACCC |
| | AACTGATCTTCAGCATCTTTTACTTTCACCAGCGTTTCTGGGTGAGCAAAAACAGGAAGGCAAAA |
| | TGCCGCAAAAAAGGGAATAAGGGCGACACGGAAATGTTGAATACTCATACTCTTCCTTTTTCAAT |
| | ATTATTGAAGCATTTATCAGGGTTATTGTCTCATGAGCGGATACATATTTGAATGTATTTAGAAA |
| | AATAAACAAATAGGGGTTCCGCGCACATTTCCCCGAAAAGTGCCACCTGACGTC |
| BLING | METDTLLLWVLLLWVPGSTGDMVFTLEDFVGDWRQTAGYNLDQVLEQGGVSSLFQNLGVSVTPIQ |
| 0.3 | RIVLSGENGLKIDIHVIIPYEGLSGDQMGQIEKIFKVVYPVDDHHFKVILHYGTLVIDGVTPNMI |
| AA | DYFGRPYEGIAVFDGKKITVTGTLWNGNKIIDERLINPDGSLLFRVTINGGGGGTSTLDKIAKNG |
| (SEQ | VIVVGHRESSVPFSYYDNQQKVVGYSQDYSNAIVEAVKKKLNKPDLQVKLIPITTQNRIPLLQNG |
| ID | TFDFECGSTTNNVERQKQAAFSDTIFVVGTRLLTKKGGDIKDFANLKDKAVVVTSGTTSEVLLNK |
| NO: | LNEEQKMNMRIISAKDHGDSFRTLESGRAVAFMMDDALLAGERAKAKKPDNWEIVGKPQSQEAYG |
| 6) | CMLRKDDPQFKKLMDDTIAQVQTSGEAEKWFDKWFKNPIPPKNLNMNFELSDEMKALFKEPNDGS |
| | GGGVTGWRLCERILLEAVGQDTQEVIVVPHSLPFKVVVISAILALVVLTIISLIILIMLWQKKPR |
| BLING | GACGGATCGGGAGATCTCCCGATCCCCTATGGTCGACTCTCAGTACAATCTGCTCTGATGCCGCA |
| 1.0 | TAGTTAAGCCAGTATCTGCTCCCTGCTTGTGTGTTGGAGGTCGCTGAGTAGTGCGCGAGCAAAAT |
| DNA | TTAAGCTACAACAAGGCAAGGCTTGACCGACAATTGCATGAAGAATCTGCTTAGGGTTAGGCGTT |
| (SEQ | TTGCGCTGCTTCGCGATGTACGGGCCAGATATACGCGTTGACATTGATTATTGACTAGTTATTAA |
| ID | TAGTAATCAATTACGGGGTCATTAGTTCATAGCCCATATATGGAGTTCCGCGTTACATAACTTAC |
| NO: | GGTAAATGGCCCGCCTGGCTGACCGCCCAACGACCCCCGCCCATTGACGTCAATAATGACGTATG |
| 7) | TTCCCATAGTAACGCCAATAGGGACTTTCCATTGACGTCAATGGGTGGACTATTTACGGTAAACT |
| | GCCCACTTGGCAGTACATCAAGTGTATCATATGCCAAGTACGCCCCCTATTGACGTCAATGACGG |
| | TAAATGGCCCGCCTGGCATTATGCCCAGTACATGACCTTATGGGACTTTCCTACTTGGCAGTACA |
| | TCTACGTATTAGTCATCGCTATTACCATGGTGATGCGGTTTTGGCAGTACATCAATGGGCGTGGA |
| | TAGCGGTTTGACTCACGGGGATTTCCAAGTCTCCACCCCATTGACGTCAATGGGAGTTTGTTTTG |
| | GCACCAAAATCAACGGGACTTTCCAAAATGTCGTAACAACTCCGCCCCATTGACGCAAATGGGCG |
| | GTAGGCGTGTACGGTGGGAGGTCTATATAAGCAGAGCTCTCTGGCTAACTAGAGAACCCACTGCT |
| | TACTGGCTTATCGAAATTAATACGACTCACTATAGGGAGACCCAAGCTGGCTAGTTAAGCTTGCC |
| | ATGGAGACAGACACACTCCTGCTATGGGTACTGCTGCTCTGGGTTCCAGGTTCCACTGGTGACAT |
| | GGTCTTCACACTCGAAGATTTCGTTGGGGACTGGCGACAGACAGCCGGCTACAACCTGGACCAAG |
| | TCCTTGAACAGGGAGGTGTGTCCAGTTTGTTTCAGAATCTCGGGGTGTCCGTAACTCCGATCCAA |
| | AGGATTGTCCTGAGCGGTGAAAATGGGCTGAAGATCGACATCCATGTCATCATCCCGTATGAAGG |
| | TTCTCCTTCTAGTACTCTGGACAAAATCGCCAAAAACGGTGTGATTGTCGTCGGTCACCGTGAAT |
| | CTTCAGTGCCTTTCTCTTATTACGACAATCAGCAAAAAGTGGTGGGTTACTCGCAGGATTACTCC |
| | AACGCCATTGTTGAAGCAGTGAAAAAGAAACTCAACAAACCGGACTTGCAGGTAAAACTGATTCC |
| | GATTACAACACAAAACCGTATTCCACTGCTGCAAAACGGCACTTTCGATTTTGAATGTGGTTCTA |
| | CCACCAACAACGTCGAACGCCAAAAACAGGCGGCTTTCTCTGACACTATTTTCGTGGTCGGTACG |
| | CGCCTGTTGACCAAAAAGGGTGGCGATATCAAAGATTTTGCCAACCTGAAAGACAAAGCCGTAGT |
| | CGTCACTTCCGGCACTACCTCTGAAGTTTTGCTCAACAAACTGAATGAAGAGCAAAAAATGAATA |
| | TGCGCATCATCAGCGCCAAAGATCACGGTGACTCTTTCCGCACCCTGGAAAGCGGTCGTGCCGTT |
| | GCCTTTATGATGGATGACGCTCTGCTGGCCGGTGAACGTGCGAAAGCGAAGAAACCAGACAACTG |
| | GGAAATCGTCGGCAAGCCGCAGTCTCAGGAGGCCTACGGTTGTATGTTGCGTAAAGATGATCCGC |
| | AGTTCAAAAAGCTGATGGATGACACCATCGCTCAGGTGCAGACCTCCGGTGAAGCGGAAAAATGG |
| | TTTGATAAGTGGTTCAAAAATCCAATTCCGCCGAAAAACCTGAACATGAATTTCGAACTGTCAGA |
| | CGAAATGAAAGCACTGTTCAAAGAACCGAATGACCCTGCTCCTGGCCTGAGCGGCGACCAAATGG |
| | GCCAGATCGAAAAAATTTTTAAGGTGGTGTACCCTGTGGATGATCATCACTTTAAGGTGATCCTG |
| | CACTATGGCACACTGGTAATCGACGGGGTTACGCCGAACATGATCGACTATTTCGGACGGCCGTA |
| | TGAAGGCATCGCCGTGTTCGACGGCAAAAAGATCACTGTAACAGGGACCCTGTGGAACGGCAACA |
| | AAATTATCGACGAGCGCCTGATCAACCCCGACGGCTCCCTGCTGTTCCGAGTAACCATCAACGGA |
| | GTGACCGGCTGGCGGCTGTGCGAACGCATTCTGGCGCTCGAGGCTGTGGGCCAGGACACGCAGGA |
| | GGTCATCGTGGTGCCACACTCCTTGCCCTTTAAGGTGGTGGTGATCTCAGCCATCCTGGCCCTGG |
| | TGGTGCTCACCATCATCTCCCTTATCATCCTCATCATGCTTTGGCAGAAGAAGCCACGTTAGTAA |
| | CATATGCTCTAGAGGGCCCGCGGTTCGAAGGTAAGCCTATCCCTAACCCTCTCCTCGGTCTCGAT |
| | TCTACGCGTACCGGTCATCATCACCATCACCATTGAGTTTAAACCCGCTGATCAGCCTCGACTGT |
| | GCCTTCTAGTTGCCAGCCATCTGTTGTTTGCCCCTCCCCCGTGCCTTCCTTGACCCTGGAAGGTG |
| | CCACTCCCACTGTCCTTTCCTAATAAAATGAGGAAATTGCATCGCATTGTCTGAGTAGGTGTCAT |
| | TCTATTCTGGGGGGTGGGGTGGGGCAGGACAGCAAGGGGGAGGATTGGGAAGACAATAGCAGGCA |
| | TGCTGGGGATGCGGTGGGCTCTATGGCTTCTGAGGCGGAAAGAACCAGCTGGGGCTCTAGGGGGT |
| | ATCCCCACGCGCCCTGTAGCGGCGCATTAAGCGCGGCGGGTGTGGTGGTTACGCGCAGCGTGACC |
| | GCTACACTTGCCAGCGCCCTAGCGCCCGCTCCTTTCGCTTTCTTCCCTTCCTTTCTCGCCACGTT |
| | CGCCGGCTTTCCCCGTCAAGCTCTAAATCGGGGCATCCCTTTAGGGTTCCGATTTAGTGCTTTAC |
| | GGCACCTCGACCCCAAAAAACTTGATTAGGGTGATGGTTCACGTAGTGGGCCATCGCCCTGATAG |
| | ACGGTTTTTCGCCCTTTGACGTTGGAGTCCACGTTCTTTAATAGTGGACTCTTGTTCCAAACTGG |
| | AACAACACTCAACCCTATCTCGGTCTATTCTTTTGATTTATAAGGGATTTTGGGGATTTCGGCCT |
| | ATTGGTTAAAAAATGAGCTGATTTAACAAAAATTTAACGCGAATTAATTCTGTGGAATGTGTGTC |
| | AGTTAGGGTGTGGAAAGTCCCCAGGCTCCCCAGGCAGGCAGAAGTATGCAAAGCATGCATCTCAA |
| | TTAGTCAGCAACCAGGTGTGGAAAGTCCCCAGGCTCCCCAGCAGGCAGAAGTATGCAAAGCATGC |
| | ATCTCAATTAGTCAGCAACCATAGTCCCGCCCCTAACTCCGCCCATCCCGCCCCTAACTCCGCCC |
| | AGTTCCGCCCATTCTCCGCCCCATGGCTGACTAATTTTTTTTATTTATGCAGAGGCCGAGGCCGC |
| | CTCTGCCTCTGAGCTATTCCAGAAGTAGTGAGGAGGCTTTTTTGGAGGCCTAGGCTTTTGCAAAA |
| | AGCTCCCGGGAGCTTGTATATCCATTTTCGGATCTGATCAAGAGACAGGATGAGGATCGTTTCGC |
| | ATGATTGAACAAGATGGATTGCACGCAGGTTCTCCGGCCGCTTGGGTGGAGAGGCTATTCGGCTA |
| | TGACTGGGCACAACAGACAATCGGCTGCTCTGATGCCGCCGTGTTCCGGCTGTCAGCGCAGGGGC |
| | GCCCGGTTCTTTTTGTCAAGACCGACCTGTCCGGTGCCCTGAATGAACTGCAGGACGAGGCAGCG |
| | CGGCTATCGTGGCTGGCCACGACGGGCGTTCCTTGCGCAGCTGTGCTCGACGTTGTCACTGAAGC |
| | GGGAAGGGACTGGCTGCTATTGGGCGAAGTGCCGGGGCAGGATCTCCTGTCATCTCACCTTGCTC |
| | CTGCCGAGAAAGTATCCATCATGGCTGATGCAATGCGGCGGCTGCATACGCTTGATCCGGCTACC |
| | TGCCCATTCGACCACCAAGCGAAACATCGCATCGAGCGAGCACGTACTCGGATGGAAGCCGGTCT |
| | TGTCGATCAGGATGATCTGGACGAAGAGCATCAGGGGCTCGCGCCAGCCGAACTGTTCGCCAGGC |
| | TCAAGGCGCGCATGCCCGACGGCGAGGATCTCGTCGTGACCCATGGCGATGCCTGCTTGCCGAAT |
| | ATCATGGTGGAAAATGGCCGCTTTTCTGGATTCATCGACTGTGGCCGGCTGGGTGTGGCGGACCG |
| | CTATCAGGACATAGCGTTGGCTACCCGTGATATTGCTGAAGAGCTTGGCGGCGAATGGGCTGACC |
| | GCTTCCTCGTGCTTTACGGTATCGCCGCTCCCGATTCGCAGCGCATCGCCTTCTATCGCCTTCTT |
| | GACGAGTTCTTCTGAGCGGGACTCTGGGGTTCGCGAAATGACCGACCAAGCGACGCCCAACCTGC |
| | CATCACGAGATTTCGATTCCACCGCCGCCTTCTATGAAAGGTTGGGCTTCGGAATCGTTTTCCGG |
| | GACGCCGGCTGGATGATCCTCCAGCGCGGGGATCTCATGCTGGAGTTCTTCGCCCACCCCAACTT |
| | GTTTATTGCAGCTTATAATGGTTACAAATAAAGCAATAGCATCACAAATTTCACAAATAAAGCAT |
| | TTTTTTCACTGCATTCTAGTTGTGGTTTGTCCAAACTCATCAATGTATCTTATCATGTCTGTATA |
| | CCGTCGACCTCTAGCTAGAGCTTGGCGTAATCATGGTCATAGCTGTTTCCTGTGTGAAATTGTTA |
| | TCCGCTCACAATTCCACACAACATACGAGCCGGAAGCATAAAGTGTAAAGCCTGGGGTGCCTAAT |
| | GAGTGAGCTAACTCACATTAATTGCGTTGCGCTCACTGCCCGCTTTCCAGTCGGGAAACCTGTCG |
| | TGCCAGCTGCATTAATGAATCGGCCAACGCGCGGGGAGAGGCGGTTTGCGTATTGGGCGCTCTTC |
| | CGCTTCCTCGCTCACTGACTCGCTGCGCTCGGTCGTTCGGCTGCGGCGAGCGGTATCAGCTCACT |
| | CAAAGGCGGTAATACGGTTATCCACAGAATCAGGGGATAACGCAGGAAAGAACATGTGAGCAAAA |
| | GGCCAGCAAAAGGCCAGGAACCGTAAAAAGGCCGCGTTGCTGGCGTTTTTCCATAGGCTCCGCCC |
| | CCCTGACGAGCATCACAAAAATCGACGCTCAAGTCAGAGGTGGCGAAACCCGACAGGACTATAAA |
| | GATACCAGGCGTTTCCCCCTGGAAGCTCCCTCGTGCGCTCTCCTGTTCCGACCCTGCCGCTTACC |
| | GGATACCTGTCCGCCTTTCTCCCTTCGGGAAGCGTGGCGCTTTCTCAATGCTCACGCTGTAGGTA |
| | TCTCAGTTCGGTGTAGGTCGTTCGCTCCAAGCTGGGCTGTGTGCACGAACCCCCCGTTCAGCCCG |
| | ACCGCTGCGCCTTATCCGGTAACTATCGTCTTGAGTCCAACCCGGTAAGACACGACTTATCGCCA |
| | CTGGCAGCAGCCACTGGTAACAGGATTAGCAGAGCGAGGTATGTAGGCGGTGCTACAGAGTTCTT |
| | GAAGTGGTGGCCTAACTACGGCTACACTAGAAGGACAGTATTTGGTATCTGCGCTCTGCTGAAGC |
| | CAGTTACCTTCGGAAAAAGAGTTGGTAGCTCTTGATCCGGCAAACAAACCACCGCTGGTAGCGGT |
| | GGTTTTTTTGTTTGCAAGCAGCAGATTACGCGCAGAAAAAAAGGATCTCAAGAAGATCCTTTGAT |
| | CTTTTCTACGGGGTCTGACGCTCAGTGGAACGAAAACTCACGTTAAGGGATTTTGGTCATGAGAT |
| | TATCAAAAAGGATCTTCACCTAGATCCTTTTAAATTAAAAATGAAGTTTTAAATCAATCTAAAGT |
| | ATATATGAGTAAACTTGGTCTGACAGTTACCAATGCTTAATCAGTGAGGCACCTATCTCAGCGAT |
| | CTGTCTATTTCGTTCATCCATAGTTGCCTGACTCCCCGTCGTGTAGATAACTACGATACGGGAGG |
| | GCTTACCATCTGGCCCCAGTGCTGCAATGATACCGCGAGACCCACGCTCACCGGCTCCAGATTTA |
| | TCAGCAATAAACCAGCCAGCCGGAAGGGCCGAGCGCAGAAGTGGTCCTGCAACTTTATCCGCCTC |
| | CATCCAGTCTATTAATTGTTGCCGGGAAGCTAGAGTAAGTAGTTCGCCAGTTAATAGTTTGCGCA |
| | ACGTTGTTGCCATTGCTACAGGCATCGTGGTGTCACGCTCGTCGTTTGGTATGGCTTCATTCAGC |
| | TCCGGTTCCCAACGATCAAGGCGAGTTACATGATCCCCCATGTTGTGCAAAAAAGCGGTTAGCTC |
| | CTTCGGTCCTCCGATCGTTGTCAGAAGTAAGTTGGCCGCAGTGTTATCACTCATGGTTATGGCAG |
| | CACTGCATAATTCTCTTACTGTCATGCCATCCGTAAGATGCTTTTCTGTGACTGGTGAGTACTCA |
| | ACCAAGTCATTCTGAGAATAGTGTATGCGGCGACCGAGTTGCTCTTGCCCGGCGTCAATACGGGA |
| | TAATACCGCGCCACATAGCAGAACTTTAAAAGTGCTCATCATTGGAAAACGTTCTTCGGGGCGAA |
| | AACTCTCAAGGATCTTACCGCTGTTGAGATCCAGTTCGATGTAACCCACTCGTGCACCCAACTGA |
| | TCTTCAGCATCTTTTACTTTCACCAGCGTTTCTGGGTGAGCAAAAACAGGAAGGCAAAATGCCGC |
| | AAAAAAGGGAATAAGGGCGACACGGAAATGTTGAATACTCATACTCTTCCTTTTTCAATATTATT |
| | GAAGCATTTATCAGGGTTATTGTCTCATGAGCGGATACATATTTGAATGTATTTAGAAAAATAAA |
| | CAAATAGGGGTTCCGCGCACATTTCCCCGAAAAGTGCCACCTGACGTC |
| BLING | METDTLLLWVLLLWVPGSTGDMVFTLEDFVGDWRQTAGYNLDQVLEQGGVSSLFQNLGVSVTPIQ |
| 1.0 | RIVLSGENGLKIDIHVIIPYEGSPSSTLDKIAKNGVIVVGHRESSVPFSYYDNQQKVVGYSQDYS |
| AA | NAIVEAVKKKLNKPDLQVKLIPITTQNRIPLLQNGTFDFECGSTTNNVERQKQAAFSDTIFVVGT |
| (SEQ | RLLTKKGGDIKDFANLKDKAVVVTSGTTSEVLLNKLNEEQKMNMRIISAKDHGDSFRTLESGRAV |
| ID | AFMMDDALLAGERAKAKKPDNWEIVGKPQSQEAYGCMLRKDDPQFKKLMDDTIAQVQTSGEAEKW |
| NO: | FDKWFKNPIPPKNLNMNFELSDEMKALFKEPNDPAPGLSGDQMGQIEKIFKVVYPVDDHHFKVIL |
| 8) | HYGTLVIDGVTPNMIDYFGRPYEGIAVFDGKKITVTGTLWNGNKIIDERLINPDGSLLFRVTING |
| | VTGWRLCERILALEAVGQDTQEVIVVPHSLPFKVVVISAILALVVLTIISLIILIMLWQKKPR |

### Microscopy

For microscopy experiments, HEK cells seeded in 12 well plates at 8×10⁵ cells per well were transfected the following day using 4 μL Lipofectamine 2000 in 100 μL Opti MEM and 2 μg DNA in 100 μL Opti MEM. The cells were then incubated overnight, trypsinized (TrypLE, Thermo), plated on Poly-D-Lysine-coated 18 mm cover slips (NeuVitro), and imaged one to three days later. Imaging was done using a Zeiss A1 Axioscope, 5×0.17NA objective, Andor iXon 888 EMCCD camera, EM gain of 600, 4×4 binning with an open optical path and microscope within a dark box. Imaging was done in a perfusion chamber with artificial cerebral spinal fluid (ACSF) using 1 μM furimazine (Promega) and heated to 37° C. ACSF was continually perfused, followed by ACSF with furimazine, followed by ACSF with furimazine and the respective concentration of glutamate, followed by a final washout with ACSF. The same ROI was used for all concentrations and statistical analysis was done using a repeated measures two-way ANOVA with Bonferroni post-hoc (n=8 per group). Images were analyzed using ImageJ for background subtraction and de-speckling to reduce noise. ROIs were selected manually for quantification.

### Example 2

### Characterization of BLING Glutamate Sensor Variants

To expand the neuroscientists' toolbox of genetically encoded indicators, bioluminescent genetically encoded neurotransmitter indicators were engineered using a multistep screening approach. In the first step, sensors were rationally designed using a variety of split luciferase variants to fuse the luciferase halves to a binding/sensing protein. Then, an automated workflow was devised to screen for improved variants in mammalian cells. Three separate versions of BLING glutamate constructs were initially created and tested based on the truncated periplasmic glutamate binding protein (Glt1) from the FRET-based glutamate sensor SuperGluSnFr and were flanked by short flexible linkers with a split luciferase “half” on each terminal. Initially, various marine luciferases were investigated that have been engineered into ultra-bright variants, do not require a cofactor to produce light, and have previously validated split sites.

BLING 0.1 was created, which included the Gaussia luciferase (GLuc) variant M43L, M110L, which is referred to as “Slow Burn GLuc” and is brighter than native GLuc. This Slow Burn (sb) GLuc variant also has glow kinetics instead of flash kinetics. The split site 105-106 that had previously been used to successfully engineer calcium indicators was used, including the 17 amino acid native secretion signal from GLuc for surface display with a PDGFRβ membrane anchor. BLING 0.2 was similarly created using NanoLuc (NLuc) at the split site 66-67, which had also been previously used to successfully engineer calcium indicators. This BLING 0.2 construct has an Igk leader sequence for cell surface display. BLING 0.3 was created with NanoLuc large and small bits split at 159-160, which has also previously been used to generate calcium indicators. All initial BLING variants produced bioluminescence with native CTZ for GLuc or h-CTZ for NLuc, with BLING 0.2 having the largest response to glutamate and being the brightest sensor (FIG. 2A and 2C).

Following the testing of the initial three BLING variants, the top performing BLING design (BLING 0.2) was further engineered and improved through linker optimization. The linkers of BLING 0.2 were replaced with 3 amino acid variable linkers that code for A, S, or P at each position (FIG. 2B). This combination of linker variants was used because they have been shown to produce diverse functionalities due to varying levels of rigidity/flexibility while significantly limiting the number of clones that need to be screened. From this library, the top performing variant was selected for further characterization (FIG. 2D). As a result of linker optimization, a

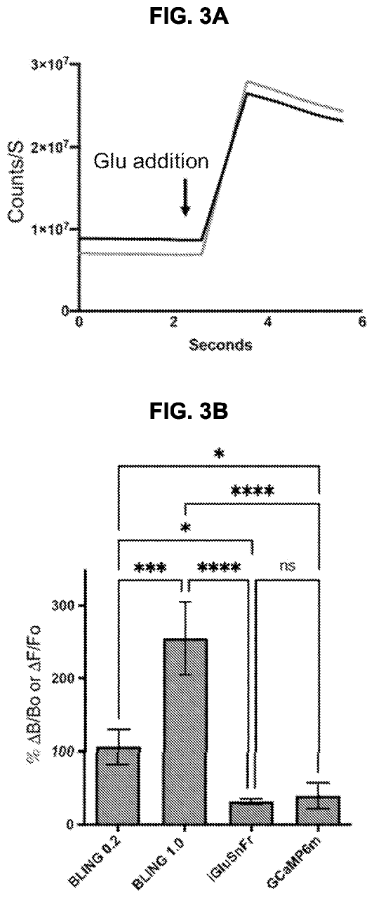

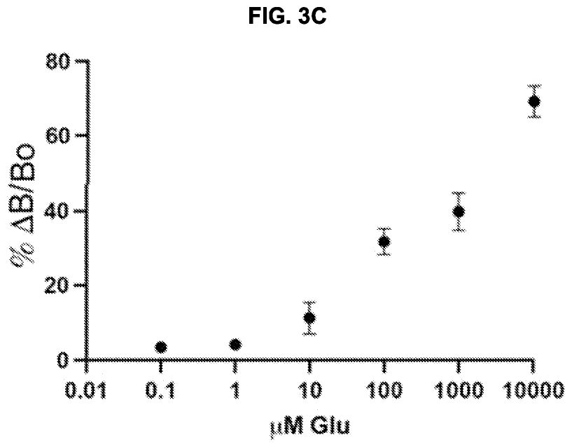

BLING variant (BLING 1.0, Addgene plasmid: 171647) was generated that has a robust response to glutamate addition when expressed in mammalian cells (FIG. 3B). BLING 1.0, derived from BLING 0.2, consistently outperformed the parental construct by 2-fold in terms of response to glutamate while maintaining its brightness using multiple plate reader modalities. However, the response amplitude can vary significantly depending on plate reading modality (FIG. 3C). It was also found that both BLING variants outperformed iGLuSnFr in terms of magnitude of percent change in response to 1 mM glutamate and GCaMP6m's response to 5 mM ionomycin when bulk measurements were conducted using a plate reader (FIG. 3B).

The dose dependent response of BLING 1.0 was then determined when expressed in mammalian cells using a photon counting plate reader (Tecan Spark). The responses to background levels of glutamate were found to be minimal (FIG. 3C). A 10.8% change in response to 10 mM glutamate was observed, with a three-fold greater response to 100 mM glutamate (31.8% change) (FIG. 3C).

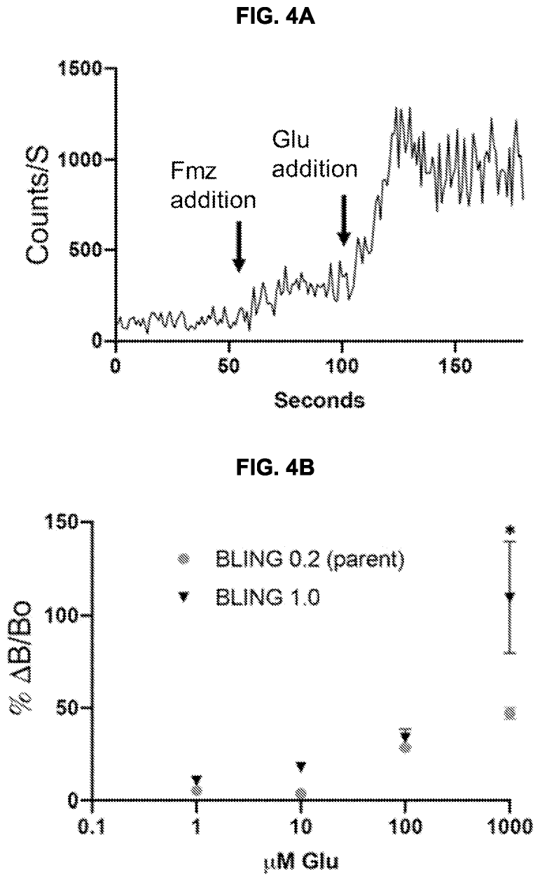

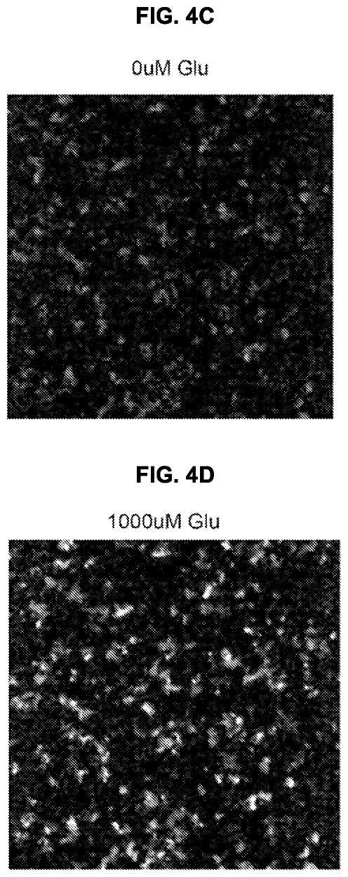

Since BLING 1.0 was found to successfully report changes in extracellular glutamate levels when recorded on the population level, it was next determined whether changes in extracellular glutamate could be observed at the single cell level. For this, live cell bioluminescence microscopy was used with HEK cells expressing BLING and varying concentrations of glutamate were perfused into the cell imaging chamber. It was found that BLING 1.0 reported changes in extracellular glutamate with responses up to 310% at the single cell level to 1 mM glutamate, and an average response of 109.6% (FIG. 4A and 4C). It was also determined that this sensor is able to report glutamate in a dose dependent manner, responding to physiological levels of glutamate and slightly outperforming BLING 0.2 (FIG. 4B).

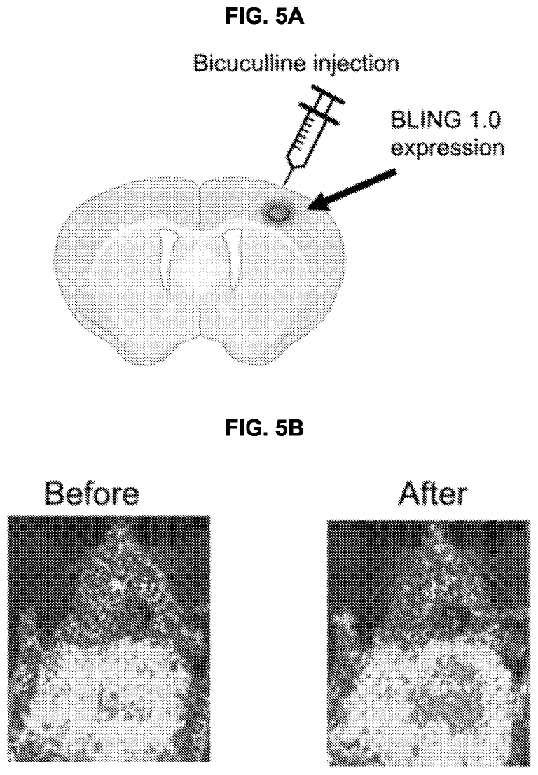

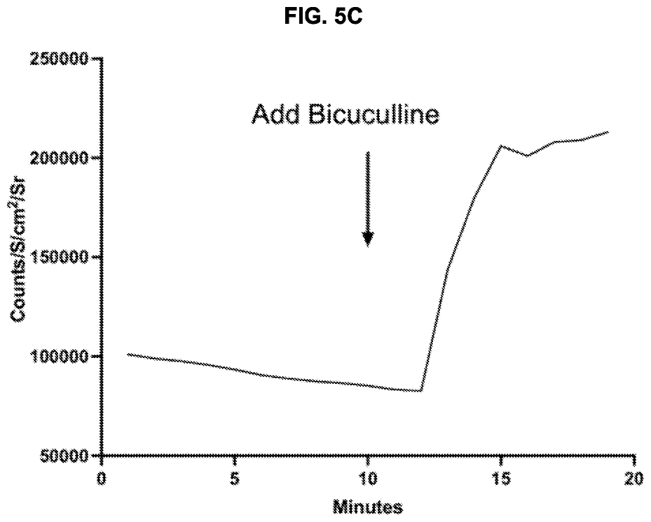

BLING 1.0 was then found to successfully report changes in glutamatergic activity in vivo using an acute seizure rat model induced by local bicuculine injection (FIG. 5A). BLING 1.0 was expressed with AAV in the sensory cortex about 2 mm below the surface of the skull (FIG. 5A). For imaging, h-CTZ was delivered intraperitoneally and rats were imaged with an IVIS Spectrum. Increases in luminescence were able to be detected following seizure induction in 4 of the 6 tested rats, with increases in luminescence over 100% (FIG. 5B-C).

These studies present the first genetically encoded bioluminescent neurotransmitter indicator, which reports changes in extracellular glutamate via changes in luminescence intensity. The bioluminescent glutamate sensors were generated based on previous work to engineer various fluorescent glutamate indicators based on the same periplasmic glutamate binding protein (Glt1), SuperGluSnFr, and iGluSnFr. In this study, the two fluorescent proteins from SuperGluSnFr were replaced with N and C terminal fragments of various marine luciferases. These indicators were found to work well to report changes in extracellular glutamate when expressed in cultured cells. This new tool opens the possibility for high throughput drug screening in cells as the bioluminescent indicators are ultra-sensitive when used in a plate reader compared to fluorescent indicators, which do not perform as well when recorded at the level of whole cell cultures (i.e., bulk measurements). In addition to neuronal recording, BLING constructs are expected to be extremely useful in situations where fluorescent indicators cannot be used, such as when screening drugs or compounds that are optically active (e.g., where the drug itself is fluorescent).

One major advantage that the disclosed bioluminescent BLING indicators present over currently existing fluorescent indicators is that the BLING sensors produce their own light and do not require an excitation light source. Excitation light can become problematic if researchers are not carefully considering the intensity of illumination, such as increasing illumination power when attempting to image deep brain areas. Overpowered excitation light can alter neuronal activity, heat tissue, lead to activation of astrocytes and microglia, cause scaring, and can lead to cell death. An additional limitation of fluorescence imaging is that many of the best fluorescent indicators currently used for optically recording neuronal activity suffer from significant photobleaching, which can severely limit the amount of continuous recording times to less than an hour in most cases. These off-target effects associated with fluorescence imaging need to be carefully considered by researchers when designing experiments. Bioluminescent indicators such as BLING can offer an orthogonal means to confirm and complement results from fluorescence-based activity studies.

Importantly, BLING is anticipated to perform well for molecular imaging of neuronal activity in deep brain structures. This class of sensors will immediately benefit ongoing research efforts to study the mechanisms that give rise to a wide array of neuronal and psychiatric disorders and provide researchers with significantly improved approaches to study neuronal activity at the level of the cell, network, and behaving animals longitudinally. Furthermore, since these reporters produce their own light, they can potentially be used as activators for light-sensitive proteins to carry out a variety of downstream functions within a cell. For example, sensors such as BLING can replace the intact luciferases in current BioLuminescent-OptoGenetic (BL-OG) constructs so the light sensitive ion channels open in response to glutamate, resulting in activity-dependent excitation or inhibition. These can then be used to improve on prior and ongoing work in neurodegenerative disorders such as spinal cord injury and Parkinson's Disease by allowing the non-invasive current stimulation of neurons to be dependent on endogenous activity.

In conclusion, a bioluminescent indicator for the neurotransmitter glutamate was developed by adapting sensing domains and split luciferases that have previously been used with success for fluorescent glutamate sensors and bioluminescent calcium sensors. The most optimized BLING glutamate sensors are already capable of reporting changes in extracellular glutamate and can serve as excellent starting points for engineering derivatives with even higher brightness and dynamic range. These bioluminescent indicators will be useful for imaging brain activity within deep brain regions. Furthermore, these sensors can be adapted and engineered for any type of neurotransmitter. These neurotransmitter sensors are adaptable for a variety of highly selective optogenetic actuators that are dependent on a specific neurotransmitter.

### Example 3

### Luciferase Variants

Bioluminescent variants based on other luciferases have also been prepared using the techniques described in Example 1. The variants utilize luciferases including Gaussia Luc, Renilla Luc, EkL9h, and alternate split sites in NanoLuc, as well as fusion combinations with mNeonGreen to shift the emission wavelength from blue to green. The NanoLuc luciferase is described by Dixon et al., ACS Chem. Biol., 11(2): 400-408 (2016), the entire contents of which are fully incorporated herein by reference. These luciferase variants may be used in combination with sensor/binding domains that are specific to any neurotransmitter, in addition to glutamate. The additional luciferase variants are shown in Table 2.

TABLE 2
Luciferase Variants

| | Sensor region/ | Wavelength |
|---|---|---|
| Luciferase | binding domain | (nm) |
| Gaussia Luc | Glt1 | ~480 |
| Renilla 8.8 535 Luc | Glt1 | ~535 |
| NanoLuc | Glt1 | ~460 |
| EkL9h | Glt1 | ~460 |
| NanoLuc + mNeonGreen | Glt1 | ~520 |
| EkL9h + mNeonGreen | Glt1 | ~520 |

* All luciferase constructs contain an IGK leader and PDFGRβ for membrane display on the cell surface, and various linker compositions and lengths, e.g., glycine and serine (flexible) or proline and alanine (rigid).

### Example 4

### Bioluminescent Dopamine Sensors

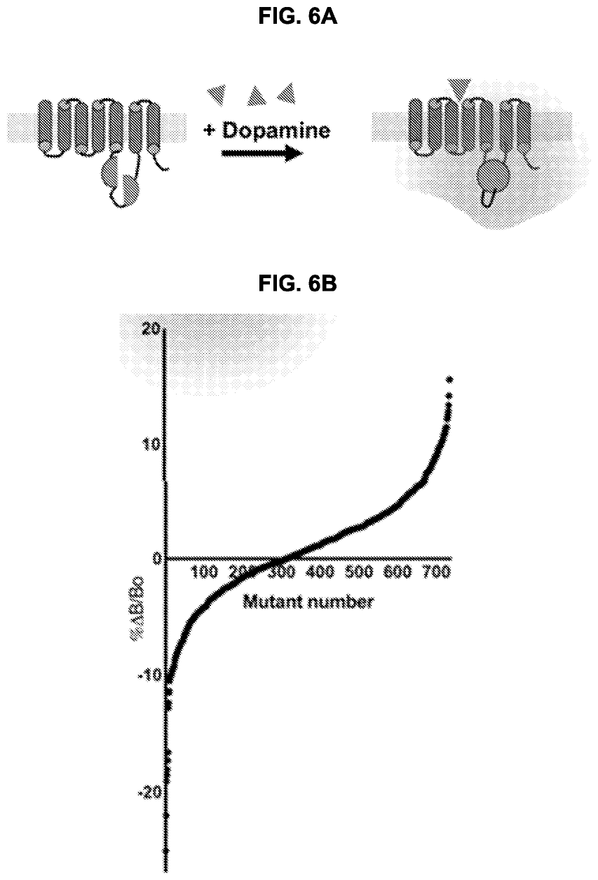

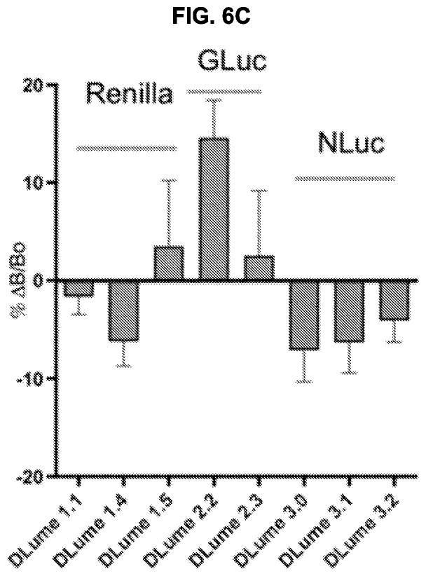

Bioluminescent variants that luminesce in the presence of the neurotransmitter dopamine have also been prepared. These dopamine variants utilize the Gaussia Luc, Renilla Luc, and alternate split sites in NanoLuc, as discussed in Example 2 and shown in Table 2. To generate the dopamine sensors, an initial library was created with circularly permutated luciferases having varied linker lengths and luciferases inserted between the S5 and S6 transmembrane domains of the human dopamine receptor (Addgene: 111053), which was previously used to create Dlight creating luminescent dopamine indicators (DLume) (FIG. 6A-B). An improved dopamine sensor variant (DLume 3.2) was created through linker mutations and library screening found it to have an approximately 20% increase in luminescence as it was the brightest DLume variant prepared (FIG. 6C). These dopamine sensors and libraries were created using the same methodologies as described in Example 1 for the BLING constructs. The DNA and amino acid sequences for the DLume 3.2 dopamine sensor variant are shown in Table 3.

TABLE 3
Bioluminescent Dopamine Sensor Polynucleotide and Amino Acid Sequences

| | |
|---|---|
| DLume | gacggatcgggagatctcccgatcccctatggtcgactctcagtacaatctgctctgat |
| 3.2 | gccgcatagttaagccagtatctgctccctgcttgtgtgttggaggtcgctgagtagtg |
| DNA | cgcgagcaaaatttaagctacaacaaggcaaggcttgaccgacaattgcatgaagaatc |
| (SEQ | tgcttagggttaggcgttttgcgctgcttcgcgatgtacgggccagatatacgcgttga |
| ID | cattgattattgactagttattaatagtaatcaattacggggtcattagttcatagccc |
| NO: | atatatggagttccgcgttacataacttacggtaaatggcccgcctggctgaccgccca |
| 9) | acgacccccgcccattgacgtcaataatgacgtatgttcccatagtaacgccaataggg |
| | actttccattgacgtcaatgggtggactatttacggtaaactgcccacttggcagtaca |
| | tcaagtgtatcatatgccaagtacgccccctattgacgtcaatgacggtaaatggcccg |
| | cctggcattatgcccagtacatgaccttatgggactttcctacttggcagtacatctac |
| | gtattagtcatcgctattaccatggtgatgcggttttggcagtacatcaatgggcgtgg |
| | atagcggtttgactcacggggatttccaagtctccaccccattgacgtcaatgggagtt |
| | tgttttggcaccaaaatcaacgggactttccaaaatgtcgtaacaactccgccccattg |
| | acgcaaatgggcggtaggcgtgtacggtgggaggtctatataagcagagctctctggct |
| | aactagagaacccactgcttactggcttatcgaaattaatacgactcactatagggaga |
| | cccaagctggctagttAAGCTTGCCccatgaagacgatcatcgccctgagctacatctt |
| | ctgcctggtgttcgccgactacaaggacgatgatgacgccatgaggactctgaacacct |
| | ctgccatggacgggactgggctggtggtggagagggacttctctgttcgtatcctcact |
| | gcctgtttcctgtcgctgctcatcctgtccacgctcctggggaacacgctggtctgtgc |
| | tgccgttatcaggttccgacacctgcggtccaaggtgaccaacttctttgtcatctcct |
| | tggctgtgtcagatctcttggtggccgtcctggtcatgccctggaaggcagtggctgag |
| | attgctggcttctggccctttgggtccttctgtaacatctgggtggcctttgacatcat |
| | gtgctccactgcatccatcctcaacctctgtgtgatcagcgtggacaggtattgggcta |
| | tctccagccctttccggtatgagagaaagatgacccccaaggcagccttcatcctgatc |
| | agtgtggcatggaccttgtctgtactcatctccttcatcccagtgcagctcagctggca |
| | caaggcaaaacccacaagcccctctgatggaaatgccacttccctggctgagaccatag |
| | acaactgtgactccagcctcagcaggacatatgccatctcatcctctgtaatcagcttt |
| | tacatccctgtggccatcatgattgtcacctacaccaggatctacaggattgctcagaa |
| | actgagctcactcattctgagcggcgaccaaatgggccagatcgaaaaaatttttaagg |
| | tggtgtaccctgtggatgatcatcactttaaggtgatcctgcactatggcacactggta |
| | atcgacggggttacgccgaacatgatcgactatttcggacggccgtatgaaggcatcgc |
| | cgtgttcgacggcaaaaagatcactgtaacagggaccctgtggaacggcaacaaaatta |
| | tcgacgagcgcctgatcaaccccgacggctccctgctgttccgagtaaccatcaacgga |
| | gtgaccggctggcggctgtgcgaacgcattctggcgGGCGGAGGCGGTAGCGGGGGAGG |
| | CGGTTCTatggtcttcacactcgaagatttcgttggggactggcgacagacagccggct |
| | acaacctggaccaagtccttgaacagggaggtgtgtccagtttgtttcagaatctcggg |
| | gtgtccgtaactccgatccaaaggattgtcctgagcggtgaaaatgggctgaagatcga |
| | catccatgtcatcatcccgtatgaaggtaatcatgaccaactgaaaagagaaactaaag |
| | tcctgaagactctgtcggtgatcatgggtgtgtttgtgtgctgttggctacctttcttc |
| | atcttgaactgcattttgcccttctgtgggtctggggagacgcagcccttctgcattga |
| | ttccaacacctttgacgtgtttgtgtggtttgggtgggctaattcatccttgaacccca |
| | tcatttatgcctttaatgctgattttcggaaggcattttcaaccctcttaggatgctac |
| | agactttgccctgcgacgaataatgccatagagacggtgagtatcaataacaatggggc |
| | cgcgatgttttccagccatcatgagccacgaggctccatctccaaggagtgcaatctgg |
| | tttacctgatcccacatgctgtgggctcctctgaggacctgaaaaaggaggaggcagct |
| | ggcatcgccagacccttggagaagctgtccccagccctatcggtcatattggactatga |
| | cactgacgtctctctggagaagatccaacccatcacacaaaacggtcagcacccaaccG |
| | GGCCCggaagcggagccactaacttctccctgttgaaacaagcaggggatgtcgaagag |
| | aatcccgggccaGCGGCCGCacccgggccaatggtgagcaagggcgaggaggtcatcaa |
| | agagttcatgcgcttcaaggtgcgcatggagggctccatgaacggccacgagttcgaga |
| | tcgagggcgagggcgagggccgcccctacgagggcacccagaccgccaagctgaaggtg |
| | accaagggcggccccctgcccttcgcctgggacatcctgtccccccagttcatgtacgg |
| | ctccaaggcgtacgtgaagcaccccgccgacatccccgattacaagaagctgtccttcc |
| | ccgagggcttcaagtgggagcgcgtgatgaacttcgaggacggcggtctggtgaccgtg |
| | acccaggactcctccctgcaggacggcacgctgatctacaaggtgaagatgcgcggcac |
| | caacttcccccccgacggccccgtaatgcagaagaagaccatgggctgggaggcctcca |
| | ccgagcgcctgtacccccgcgacggcgtgctgaagggcgagatccaccaggccctgaag |
| | ctgaaggacggcggccactacctggtggagttcaagaccatctacatggccaagaagcc |
| | cgtgcaactgcccggctactactacgtggacaccaagctggacatcacctcccacaacg |
| | aggactacaccatcgtggaacagtacgagcgctccgagggccgccaccacctgttcctg |
| | tacggcatggacgagcTGTACAagTAAcatatgcTCTAGAgggcccgcggttcgaaggt |
| | aagcctatccctaaccctctcctcggtctcgattctacgcgtaccggtcatcatcacca |
| | tcaccattgagtttaaacccgctgatcagcctcgactgtgccttctagttgccagccat |
| | ctgttgtttgcccctcccccgtgccttccttgaccctggaaggtgccactcccactgtc |
| | ctttcctaataaaatgaggaaattgcatcgcattgtctgagtaggtgtcattctattct |
| | ggggggtggggtggggcaggacagcaagggggaggattgggaagacaatagcaggcatg |
| | ctggggatgcggtgggctctatggcttctgaggcggaaagaaccagctggggctctagg |
| | gggtatccccacgcgccctgtagcggcgcattaagcgcggcgggtgtggtggttacgcg |
| | cagcgtgaccgctacacttgccagcgccctagcgcccgctcctttcgctttcttccctt |
| | cctttctcgccacgttcgccggctttccccgtcaagctctaaatcggggcatcccttta |
| | gggttccgatttagtgctttacggcacctcgaccccaaaaaacttgattagggtgatgg |
| | ttcacgtagtgggccatcgccctgatagacggtttttcgccctttgacgttggagtcca |
| | cgttctttaatagtggactcttgttccaaactggaacaacactcaaccctatctcggtc |
| | tattcttttgatttataagggattttggggatttcggcctattggttaaaaaatgagct |
| | gatttaacaaaaatttaacgcgaattaattctgtggaatgtgtgtcagttagggtgtgg |
| | aaagtccccaggctccccaggcaggcagaagtatgcaaagcatgcatctcaattagtca |
| | gcaaccaggtgtggaaagtccccaggctccccagcaggcagaagtatgcaaagcatgca |
| | tctcaattagtcagcaaccatagtcccgcccctaactccgcccatcccgcccctaactc |
| | cgcccagttccgcccattctccgccccatggctgactaattttttttatttatgcagag |
| | gccgaggccgcctctgcctctgagctattccagaagtagtgaggaggcttttttggagg |
| | cctaggcttttgcaaaaagctcccgggagcttgtatatccattttcggatctgatcaag |
| | agacaggatgaggatcgtttcgcatgattgaacaagatggattgcacgcaggttctccg |
| | gccgcttgggtggagaggctattcggctatgactgggcacaacagacaatcggctgctc |
| | tgatgccgccgtgttccggctgtcagcgcaggggcgcccggttctttttgtcaagaccg |
| | acctgtccggtgccctgaatgaactgcaggacgaggcagcgcggctatcgtggctggcc |
| | acgacgggcgttccttgcgcagctgtgctcgacgttgtcactgaagcgggaagggactg |
| | gctgctattgggcgaagtgccggggcaggatctcctgtcatctcaccttgctcctgccg |
| | agaaagtatccatcatggctgatgcaatgcggcggctgcatacgcttgatccggctacc |
| | tgcccattcgaccaccaagcgaaacatcgcatcgagcgagcacgtactcggatggaagc |
| | cggtcttgtcgatcaggatgatctggacgaagagcatcaggggctcgcgccagccgaac |
| | tgttcgccaggctcaaggcgcgcatgcccgacggcgaggatctcgtcgtgacccatggc |
| | gatgcctgcttgccgaatatcatggtggaaaatggccgcttttctggattcatcgactg |
| | tggccggctgggtgtggcggaccgctatcaggacatagcgttggctacccgtgatattg |
| | ctgaagagcttggcggcgaatgggctgaccgcttcctcgtgctttacggtatcgccgct |
| | cccgattcgcagcgcatcgccttctatcgccttcttgacgagttcttctgagcgggact |
| | ctggggttcgcgaaatgaccgaccaagcgacgcccaacctgccatcacgagatttcgat |
| | tccaccgccgccttctatgaaaggttgggcttcggaatcgttttccgggacgccggctg |
| | gatgatcctccagcgcggggatctcatgctggagttcttcgcccaccccaacttgttta |
| | ttgcagcttataatggttacaaataaagcaatagcatcacaaatttcacaaataaagca |
| | tttttttcactgcattctagttgtggtttgtccaaactcatcaatgtatcttatcatgt |
| | ctgtataccgtcgacctctagctagagcttggcgtaatcatggtcatagctgtttcctg |
| | tgtgaaattgttatccgctcacaattccacacaacatacgagccggaagcataaagtgt |
| | aaagcctggggtgcctaatgagtgagctaactcacattaattgcgttgcgctcactgcc |
| | cgctttccagtcgggaaacctgtcgtgccagctgcattaatgaatcggccaacgcgcgg |
| | ggagaggcggtttgcgtattgggcgctcttccgcttcctcgctcactgactcgctgcgc |
| | tcggtcgttcggctgcggcgagcggtatcagctcactcaaaggcggtaatacggttatc |
| | cacagaatcaggggataacgcaggaaagaacatgtgagcaaaaggccagcaaaaggcca |
| | ggaaccgtaaaaaggccgcgttgctggcgtttttccataggctccgcccccctgacgag |
| | catcacaaaaatcgacgctcaagtcagaggtggcgaaacccgacaggactataaagata |
| | ccaggcgtttccccctggaagctccctcgtgcgctctcctgttccgaccctgccgctta |
| | ccggatacctgtccgcctttctcccttcgggaagcgtggcgctttctcaatgctcacgc |
| | tgtaggtatctcagttcggtgtaggtcgttcgctccaagctgggctgtgtgcacgaacc |
| | ccccgttcagcccgaccgctgcgccttatccggtaactatcgtcttgagtccaacccgg |
| | taagacacgacttatcgccactggcagcagccactggtaacaggattagcagagcgagg |
| | tatgtaggcggtgctacagagttcttgaagtggtggcctaactacggctacactagaag |
| | gacagtatttggtatctgcgctctgctgaagccagttaccttcggaaaaagagttggta |
| | gctcttgatccggcaaacaaaccaccgctggtagcggtggtttttttgtttgcaagcag |
| | cagattacgcgcagaaaaaaaggatctcaagaagatcctttgatcttttctacggggtc |
| | tgacgctcagtggaacgaaaactcacgttaagggattttggtcatgagattatcaaaaa |
| | ggatcttcacctagatccttttaaattaaaaatgaagttttaaatcaatctaaagtata |
| | tatgagtaaacttggtctgacagttaccaatgcttaatcagtgaggcacctatctcagc |
| | gatctgtctatttcgttcatccatagttgcctgactccccgtcgtgtagataactacga |
| | tacgggagggcttaccatctggccccagtgctgcaatgataccgcgagacccacgctca |
| | ccggctccagatttatcagcaataaaccagccagccggaagggccgagcgcagaagtgg |
| | tcctgcaactttatccgcctccatccagtctattaattgttgccgggaagctagagtaa |
| | gtagttcgccagttaatagtttgcgcaacgttgttgccattgctacaggcatcgtggtg |
| | tcacgctcgtcgtttggtatggcttcattcagctccggttcccaacgatcaaggcgagt |
| | tacatgatcccccatgttgtgcaaaaaagcggttagctccttcggtcctccgatcgttg |
| | tcagaagtaagttggccgcagtgttatcactcatggttatggcagcactgcataattct |
| | cttactgtcatgccatccgtaagatgcttttctgtgactggtgagtactcaaccaagtc |
| | attctgagaatagtgtatgcggcgaccgagttgctcttgcccggcgtcaatacgggata |
| | ataccgcgccacatagcagaactttaaaagtgctcatcattggaaaacgttcttcgggg |
| | cgaaaactctcaaggatcttaccgctgttgagatccagttcgatgtaacccactcgtgc |
| | acccaactgatcttcagcatcttttactttcaccagcgtttctgggtgagcaaaaacag |
| | gaaggcaaaatgccgcaaaaaagggaataagggcgacacggaaatgttgaatactcata |
| | ctcttcctttttcaatattattgaagcatttatcagggttattgtctcatgagcggata |
| | catatttgaatgtatttagaaaaataaacaaataggggttccgcgcacatttccccgaa |
| | aagtgccacctgacgtc |
| DLume | MKTIIALSYIFCLVFADYKDDDDAMRTLNTSAMDGTGLVVERDFSVRILTACFLSLLIL |
| 3.2 | STLLGNTLVCAAVIRFRHLRSKVTNFFVISLAVSDLLVAVLVMPWKAVAEIAGFWPFGS |
| AA | FCNIWVAFDIMCSTASILNLCVISVDRYWAISSPFRYERKMTPKAAFILISVAWTLSVL |
| (SEQ | ISFIPVQLSWHKAKPTSPSDGNATSLAETIDNCDSSLSRTYAISSSVISFYIPVAIMIV |
| ID | TYTRIYRIAQKLSSLILSGDQMGQIEKIFKVVYPVDDHHFKVILHYGTLVIDGVTPNMI |
| NO: | DYFGRPYEGIAVFDGKKITVTGTLWNGNKIIDERLINPDGSLLFRVTINGVTGWRLCER |
| 10) | ILAGGGGSGGGGSMVFTLEDFVGDWRQTAGYNLDQVLEQGGVSSLFQNLGVSVTPIQRI |
| | VLSGENGLKIDIHVIIPYEGNHDQLKRETKVLKTLSVIMGVFVCCWLPFFILNCILPFC |
| | GSGETQPFCIDSNTFDVFVWFGWANSSLNPIIYAFNADFRKAFSTLLGCYRLCPATNNA |
| | IETVSINNNGAAMFSSHHEPRGSISKECNLVYLIPHAVGSSEDLKKEEAAGIARPLEKL |
| | SPALSVILDYDTDVSLEKIQPITQNGQHPTGP |

Example 5

Bioluminescent Acetylcholine and GABA Sensors

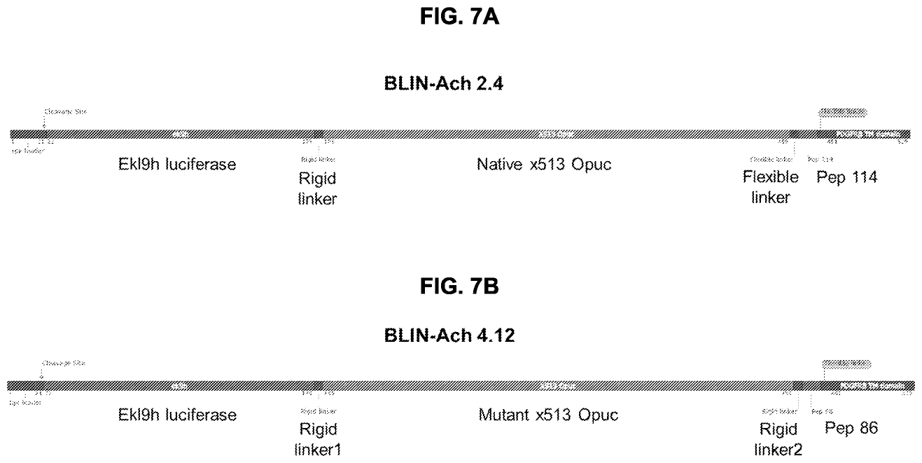

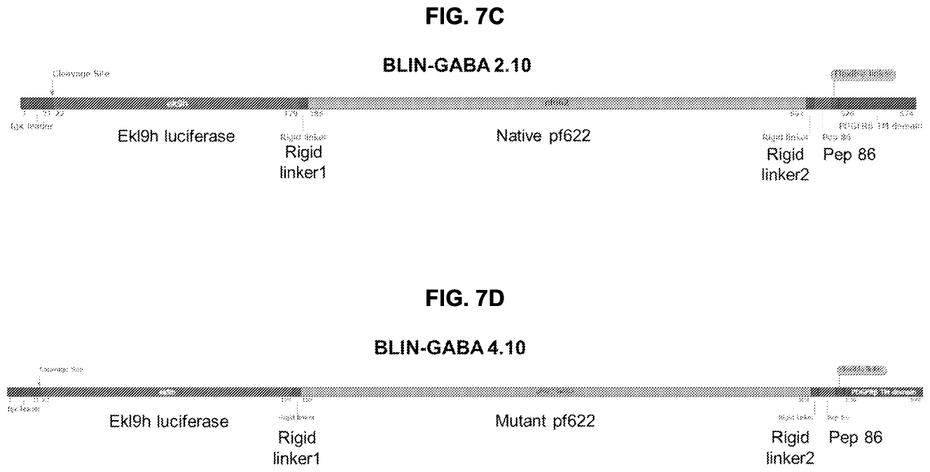

Bioluminescent sensor variants that luminesce in the presence of the neurotransmitters acetylcholine (Ach) and GABA have also been prepared (FIG. 7A-D). These neurotransmitter sensor variants utilize an N-terminal Ek19H luciferase (shown in Table 2) or a NanoLuc small bit (smBit) luciferase and were created using the same methodologies as described in Example 1 for the BLING glutamate constructs.

The BLIN-Ach 2.4 acetylcholine variant comprises a rigid linker, the native Thermoanaerobacter sp. X513 OpuBC sequence (RefSeq No. WP_009052931.1), a flexible linker, and Pep 114, Pep 86, or native peptide (FIG. 7A). The X513 OpuBC from Thermoanaerobacter sp. is an acetylcholine binding protein. The BLIN-Ach 4.12 acetylcholine variant comprises a first rigid linker, a mutant Thermoanaerobacter sp. X513 OpuBC sequence, a second rigid linker, and Pep 114, Pep 86, or native peptide (FIG. 7B). The mutant Thermoanaerobacter sp. X513 OpuBC sequence is described by Borden et al., BioRxiv, doi: 10.1101/2020.02.07.939504 (2020), the entire contents of which are fully incorporated herein by reference. In addition, a BLIN-Ach 4.10 acetylcholine variant was prepared and found to exhibit the best luminescence properties from an Ach sensor library. The DNA and amino acid sequences for these different Ach variants are shown in Table 4.

The BLIN-GABA 2.10 GABA variant comprises a first rigid linker, the native Pseudomonas fl. pf622 sequence (RefSeq No. WP_078824171.1), a second rigid linker, and Pep 86 (FIG. 7C).

The pf622 from Pseudomonas fl. is a GABA binding protein. The BLIN-GABA 4.10 GABA variant comprises a first rigid linker, a mutant Pseudomonas fl. pf622 sequence (N260A), a second rigid linker, and Pep 114, Pep 86, or native peptide (FIG. 7D). The mutant Pseudomonas fl. pf622 sequence (N260A) is described by Marvin et al., Nature Methods, 16 (8): 763-770 (2019), the entire contents of which are fully incorporated herein by reference. In addition, a BLIN-GABA 4.11 GABA variant was prepared and found to exhibit the best luminescence properties from a GABA sensor library. The DNA and amino acid sequences for these different GABA variants are shown in Table 4.

TABLE 4
BLIN (Ach and GABA) Variant Polynucleotide and Amino Acid Sequences

| | |
|---|---|
| BLIN- | ATGGAGACAGACACACTCCTGCTATGGGTACTGCTGCTCTGGGTTCCAGGTTCCACTGG |
| Ach | TGACATGGTTTTCACCCTGGAAGACTTCGTTGGTGACTGGGAGCAGACCGCTGCTTACA |
| 2.4 | ACCTGGACCAGGTTCTGGAACAGGGTGGTGTGTCTTCTGTGCTCCAGACGCTGGCTGTT |
| DNA | TCTGTGACTCCGATCCAGCGTATCGTTCGTTCTGGTGAAAACGGTCTGAAAATCGACAT |
| (SEQ | CCACGTTATCATCCCGTACGAAGGTCTGTCTGCTGATCAGATGGCTCATATCGAAGAGG |
| ID | TCTTCAAAGTTGTTTACCCGGTTGACGACCACCACTTCAAAGTCATCATGGAGTACGGT |
| NO: | ACCCTGGTTATCGACGGTGTTACCCCGAACATGCTCAACTACTTCGGTCGTCCGTACGA |
| 11) | GGGTATCGCTGTTTTCGACGGTAAGAAGATCACCGTTACCGGTACCCTGTGGAACGGTA |
| | ACAAAATCATCGACGAACGTCTGATCACCCCGGACGGTTCTATGCTGTTCCGTGTTACG |
| | ATCAACCCGGCACCAGCGCCGGCAAATGACACAGTCGTTGTCGGAAGCAAGAATTTCAC |
| | GGAACAAATAATCGTTGCCAATATGGTTGCTGAGATGATCGAAGCGCACACCGACCTTA |
| | AAGTTGTACGCAAGTTGAACTTGGGCGGAACGAATGTTAATTTTGAGGCGATAAAACGA |
| | GGCGGAGCCAATAACGGTATAGACATATATGTAGAGTATACTGGAACCGGGCTGGTCGA |
| | TATTCTCGGGATGGAACCAACGACTGATCCAGAAAAAGCTTATGAAACTGTTAAAAAGG |
| | AATATAAGGATAAATGGAATATCGTGTGGCTCAAGCCACTCGGTTTCAATAATACCTAC |
| | ACTCTTGCTGTCAAGGATGAGTTGGCGAAACAATATAATCTTAAAACGTTCTCAGACTT |
| | GGCGAAAATAAGTGACAAGCTGATCCTCGGAGCTACGATGGAGTTTTTGGAGCGGCCAG |
| | ACGGCTACCCCGGATTGCAAAAGGTATATAACTTTAAATTTAAGCATACCAAGTCTATG |
| | GATATGGGCATCCGCTACACCGCCATTGACAACAACGAAGTGCAGGTTATTGACGCTTT |
| | CGCTACGGACGGGCTCCTCGTTAGCCACAAATTGAAGATCCTGGAAGATGATAAGCATT |
| | TTTTTCCACCGTATTATGCAGCGCCAATAATAAGACAAGATGTGCTGGATAAGCATCCG |
| | GAGCTTAAGGACGTTTTGAATAAGCTTGCCAACCAAATTAGTGATGAAGAAATGCAAAA |
| | ATTGAATTATAAGGTGGATGGTGAAGGTCAGGACCCAGCTAAGGTCGCCAAGGAGTTCC |
| | TGAAGGAGAAGGGACTGATCGGATCCGGAGGCGGCGTGACGGGATACAGACTGTTCGAG |
| | GAGATCCTTGGAGGCGGTGGCAGCGCTGTGGGCCAGGACACGCAGGAGGTCATCGTGGT |
| | GCCACACTCCTTGCCCTTTAAGGTGGTGGTGATCTCAGCCATCCTGGCCCTGGTGGTGC |
| | TCACCATCATCTCCCTTATCATCCTCATCATGCTTTGGCAGAAGAAGCCACGTTAG |
| BLIN- | METDTLLLWVLLLWVPGSTGDMVFTLEDFVGDWEQTAAYNLDQVLEQGGVSSVLQTLAV |
| Ach | SVTPIQRIVRSGENGLKIDIHVIIPYEGLSADQMAHIEEVFKVVYPVDDHHFKVIMEYG |
| 2.4 | TLVIDGVTPNMLNYFGRPYEGIAVFDGKKITVTGTLWNGNKIIDERLITPDGSMLFRVT |
| AA | INPAPAPANDTVVVGSKNFTEQIIVANMVAEMIEAHTDLKVVRKLNLGGTNVNFEAIKR |
| (SEQ | GGANNGIDIYVEYTGTGLVDILGMEPTTDPEKAYETVKKEYKDKWNIVWLKPLGFNNTY |
| ID | TLAVKDELAKQYNLKTFSDLAKISDKLILGATMEFLERPDGYPGLQKVYNFKFKHTKSM |
| NO: | DMGIRYTAIDNNEVQVIDAFATDGLLVSHKLKILEDDKHFFPPYYAAPIIRQDVLDKHP |
| 12) | ELKDVLNKLANQISDEEMQKLNYKVDGEGQDPAKVAKEFLKEKGLIGSGGGVTGYRLFE |
| | EILGGGGSAVGQDTQEVIVVPHSLPFKVVVISAILALVVLTIISLIILIMLWQKKPR* |
| BLIN- | gacggatcgggagatctcccgatcccctatggtcgactctcagtacaatctgctctgat |
| Ach | gccgcatagttaagccagtatctgctccctgcttgtgtgttggaggtcgctgagtagtg |
| 4.10 | cgcgagcaaaatttaagctacaacaaggcaaggcttgaccgacaattgcatgaagaatc |
| DNA | tgcttagggttaggcgttttgcgctgcttcgcgatgtacgggccagatatacgcgttga |
| (SEQ | cattgattattgactagttattaatagtaatcaattacggggtcattagttcatagccc |
| ID | atatatggagttccgcgttacataacttacggtaaatggcccgcctggctgaccgccca |
| NO: | acgacccccgcccattgacgtcaataatgacgtatgttcccatagtaacgccaataggg |
| 13) | actttccattgacgtcaatgggtggactatttacggtaaactgcccacttggcagtaca |
| | tcaagtgtatcatatgccaagtacgccccctattgacgtcaatgacggtaaatggcccg |
| | cctggcattatgcccagtacatgaccttatgggactttcctacttggcagtacatctac |
| | gtattagtcatcgctattaccatggtgatgcggttttggcagtacatcaatgggcgtgg |
| | atagcggtttgactcacggggatttccaagtctccaccccattgacgtcaatgggagtt |
| | tgttttggcaccaaaatcaacgggactttccaaaatgtcgtaacaactccgccccattg |
| | acgcaaatgggcggtaggcgtgtacggtgggaggtctatataagcagagctctctggct |
| | aactagagaacccactgcttactggcttatcgaaattaatacgactcactatagggaga |
| | cccaagctggctagttAAGCTTGCCatggagacagacacactcctgctatgggtactgc |
| | tgctctgggttccaggttccactggtgacatggttttcaccctggaggacttcgttggt |
| | gactgggagcagaccgctgcttacaacctggaccaggttctggaacagggtggtgtgtc |
| | ttctgtgctccagacgctggctgtttctgtgactccgatccagcgtatcgttcgttctg |
| | gtgaaaacggtctgaaaatcgacatccacgttatcatcccgtacgaaggtctgtctgct |
| | gatcagatggctcatatcgaagaggtcttcaaagttgtttacccggttgacgaccacca |
| | cttcaaagtcatcatggagtacggtaccctggttatcgacggtgttaccccgaacatgc |
| | tcaactacttcggtcgtccgtacgagggtatcgctgttttcgacggtaagaagatcacc |
| | gttaccggtaccctgtggaacggtaacaaaatcatcgacgaacgtctgatcaccccgga |
| | cggttctatgctgttccgtgttacgatcaacCCGGCACCAGCGCCGGCAAATGACACAG |
| | TCGTTGTCGGAATCATTAATTTCACGGAAGGGATAATCGTTGCCAATATGGTTGCTGAG |
| | ATGATCGAAGCGCACACCGACCTTAAAGTTGTACGCAAGTTGAACTTGGGCGGAGTAAA |
| | TGTTAATTTTGAGGCGATAAAACGAGGCGGAGCCAATAACGGTATAGACATATATGTAG |
| | AGTATACTGGACATGGGCTGGTCGATATTCTCGGGTTCCCTGCAACGACTGATCCAGAA |
| | GGTGCTTATGAAACTGTTAAAAAGGAATATAGGGATAAATGGAATATCGTGTGGCTCAA |
| | GCCACTCGGTTTCAATAATACCTACACTCTTACCGTCAAGGATGAGTTGGCGAAACAAT |
| | ATAATCTTAAAACGTTCTCAGACTTGGCGAAAATAAGTGACAAGCTGATCCTCGGAGCT |
| | ACGATGTTCTTTTTGGAGGGTCCAGACGGCTACCCCGGATTGCAAAAGTTGTATAACTT |
| | TAAATTTAAGCATACCAAGTCTATGGATATGGGCATCCGCTACACCGCCATTGACAACA |
| | ACGAAGTGCAGGTTATTGACGCTTGGGCTACGGACGGGCTCCTCGTTAGCCACAAATTG |
| | AAGATCCTGGAAGATGATAAGGCTTTTTTTCCACCGTATTATGCAGCGCCAATAATAAG |
| | ACAAGATGTGCTGGATAAGCATCCGGAGCTTAAGGACGTTTTGAATAAGCTTGCCAACC |
| | AAATTAGTCTGGAAGAAATGCAAAAATTGAATTATAAGGTGGATGGTGAAGGTCAGGAC |
| | CCAGCTAAGGTCGCCAAGGAGTTCCTGAAGGAGAAGGGACTGATCCCTGCTCCAGCACC |
| | AGTGTCCggctggcggctgttcAAGAAAattTCTGGAGGCGGTGGCAGCgctgtgggcc |
| | aggacacgcaggaggtcatcgtggtgccacactccttgccctttaaggtggtggtgatc |
| | tcagccatcctggccctggtggtgctcaccatcatctcccttatcatcctcatcatgct |
| | ttggcagaagaagccacgttagTAAcatatgcTCTAGAgggcccgcggttcgaaggtaa |
| | gcctatccctaaccctctcctcggtctcgattctacgcgtaccggtcatcatcaccatc |
| | accattgagtttaaacccgctgatcagcctcgactgtgccttctagttgccagccatct |
| | gttgtttgcccctcccccgtgccttccttgaccctggaaggtgccactcccactgtcct |
| | ttcctaataaaatgaggaaattgcatcgcattgtctgagtaggtgtcattctattctgg |
| | ggggtggggtggggcaggacagcaagggggaggattgggaagacaatagcaggcatgct |
| | ggggatgcggtgggctctatggcttctgaggcggaaagaaccagctggggctctagggg |
| | gtatccccacgcgccctgtagcggcgcattaagcgcggcgggtgtggtggttacgcgca |
| | gcgtgaccgctacacttgccagcgccctagcgcccgctcctttcgctttcttcccttcc |
| | tttctcgccacgttcgccggctttccccgtcaagctctaaatcggggcatccctttagg |
| | gttccgatttagtgctttacggcacctcgaccccaaaaaacttgattagggtgatggtt |
| | cacgtagtgggccatcgccctgatagacggtttttcgccctttgacgttggagtccacg |
| | ttctttaatagtggactcttgttccaaactggaacaacactcaaccctatctcggtcta |
| | ttcttttgatttataagggattttggggatttcggcctattggttaaaaaatgagctga |
| | tttaacaaaaatttaacgcgaattaattctgtggaatgtgtgtcagttagggtgtggaa |
| | agtccccaggctccccaggcaggcagaagtatgcaaagcatgcatctcaattagtcagc |
| | aaccaggtgtggaaagtccccaggctccccagcaggcagaagtatgcaaagcatgcatc |
| | tcaattagtcagcaaccatagtcccgcccctaactccgcccatcccgcccctaactccg |
| | cccagttccgcccattctccgccccatggctgactaattttttttatttatgcagaggc |
| | cgaggccgcctctgcctctgagctattccagaagtagtgaggaggcttttttggaggcc |
| | taggcttttgcaaaaagctcccgggagcttgtatatccattttcggatctgatcaagag |
| | acaggatgaggatcgtttcgcatgattgaacaagatggattgcacgcaggttctccggc |
| | cgcttgggtggagaggctattcggctatgactgggcacaacagacaatcggctgctctg |
| | atgccgccgtgttccggctgtcagcgcaggggcgcccggttctttttgtcaagaccgac |
| | ctgtccggtgccctgaatgaactgcaggacgaggcagcgcggctatcgtggctggccac |
| | gacgggcgttccttgcgcagctgtgctcgacgttgtcactgaagcgggaagggactggc |
| | tgctattgggcgaagtgccggggcaggatctcctgtcatctcaccttgctcctgccgag |
| | aaagtatccatcatggctgatgcaatgcggcggctgcatacgcttgatccggctacctg |
| | cccattcgaccaccaagcgaaacatcgcatcgagcgagcacgtactcggatggaagccg |
| | gtcttgtcgatcaggatgatctggacgaagagcatcaggggctcgcgccagccgaactg |
| | ttcgccaggctcaaggcgcgcatgcccgacggcgaggatctcgtcgtgacccatggcga |
| | tgcctgcttgccgaatatcatggtggaaaatggccgcttttctggattcatcgactgtg |
| | gccggctgggtgtggcggaccgctatcaggacatagcgttggctacccgtgatattgct |
| | gaagagcttggcggcgaatgggctgaccgcttcctcgtgctttacggtatcgccgctcc |
| | cgattcgcagcgcatcgccttctatcgccttcttgacgagttcttctgagcgggactct |
| | ggggttcgcgaaatgaccgaccaagcgacgcccaacctgccatcacgagatttcgattc |
| | caccgccgccttctatgaaaggttgggcttcggaatcgttttccgggacgccggctgga |
| | tgatcctccagcgcggggatctcatgctggagttcttcgcccaccccaacttgtttatt |
| | gcagcttataatggttacaaataaagcaatagcatcacaaatttcacaaataaagcatt |
| | tttttcactgcattctagttgtggtttgtccaaactcatcaatgtatcttatcatgtct |
| | gtataccgtcgacctctagctagagcttggcgtaatcatggtcatagctgtttcctgtg |
| | tgaaattgttatccgctcacaattccacacaacatacgagccggaagcataaagtgtaa |
| | agcctggggtgcctaatgagtgagctaactcacattaattgcgttgcgctcactgcccg |
| | ctttccagtcgggaaacctgtcgtgccagctgcattaatgaatcggccaacgcgcgggg |
| | agaggcggtttgcgtattgggcgctcttccgcttcctcgctcactgactcgctgcgctc |
| | ggtcgttcggctgcggcgagcggtatcagctcactcaaaggcggtaatacggttatcca |
| | cagaatcaggggataacgcaggaaagaacatgtgagcaaaaggccagcaaaaggccagg |
| | aaccgtaaaaaggccgcgttgctggcgtttttccataggctccgcccccctgacgagca |
| | tcacaaaaatcgacgctcaagtcagaggtggcgaaacccgacaggactataaagatacc |
| | aggcgtttccccctggaagctccctcgtgcgctctcctgttccgaccctgccgcttacc |
| | ggatacctgtccgcctttctcccttcgggaagcgtggcgctttctcaatgctcacgctg |
| | taggtatctcagttcggtgtaggtcgttcgctccaagctgggctgtgtgcacgaacccc |
| | ccgttcagcccgaccgctgcgccttatccggtaactatcgtcttgagtccaacccggta |
| | agacacgacttatcgccactggcagcagccactggtaacaggattagcagagcgaggta |
| | tgtaggcggtgctacagagttcttgaagtggtggcctaactacggctacactagaagga |
| | cagtatttggtatctgcgctctgctgaagccagttaccttcggaaaaagagttggtagc |
| | tcttgatccggcaaacaaaccaccgctggtagcggtggtttttttgtttgcaagcagca |
| | gattacgcgcagaaaaaaaggatctcaagaagatcctttgatcttttctacggggtctg |
| | acgctcagtggaacgaaaactcacgttaagggattttggtcatgagattatcaaaaagg |
| | atcttcacctagatccttttaaattaaaaatgaagttttaaatcaatctaaagtatata |
| | tgagtaaacttggtctgacagttaccaatgcttaatcagtgaggcacctatctcagcga |
| | tctgtctatttcgttcatccatagttgcctgactccccgtcgtgtagataactacgata |
| | cgggagggcttaccatctggccccagtgctgcaatgataccgcgagacccacgctcacc |
| | ggctccagatttatcagcaataaaccagccagccggaagggccgagcgcagaagtggtc |
| | ctgcaactttatccgcctccatccagtctattaattgttgccgggaagctagagtaagt |
| | agttcgccagttaatagtttgcgcaacgttgttgccattgctacaggcatcgtggtgtc |
| | acgctcgtcgtttggtatggcttcattcagctccggttcccaacgatcaaggcgagtta |
| | catgatcccccatgttgtgcaaaaaagcggttagctccttcggtcctccgatcgttgtc |
| | agaagtaagttggccgcagtgttatcactcatggttatggcagcactgcataattctct |
| | tactgtcatgccatccgtaagatgcttttctgtgactggtgagtactcaaccaagtcat |
| | tctgagaatagtgtatgcggcgaccgagttgctcttgcccggcgtcaatacgggataat |
| | accgcgccacatagcagaactttaaaagtgctcatcattggaaaacgttcttcggggcg |
| | aaaactctcaaggatcttaccgctgttgagatccagttcgatgtaacccactcgtgcac |
| | ccaactgatcttcagcatcttttactttcaccagcgtttctgggtgagcaaaaacagga |
| | aggcaaaatgccgcaaaaaagggaataagggcgacacggaaatgttgaatactcatact |
| | cttcctttttcaatattattgaagcatttatcagggttattgtctcatgagcggataca |
| | tatttgaatgtatttagaaaaataaacaaataggggttccgcgcacatttccccgaaaa |
| | gtgccacctgacgtc |
| BLIN- | METDTLLLWVLLLWVPGSTGDMVFTLEDFVGDWEQTAAYNLDQVLEQGGVSSVLQTLAV |
| Ach | SVTPIQRIVRSGENGLKIDIHVIIPYEGLSADQMAHIEEVEKVVYPVDDHHFKVIMEYG |
| 4.10 | TLVIDGVTPNMLNYFGRPYEGIAVFDGKKITVTGTLWNGNKIIDERLITPDGSMLFRVT |
| AA | INPAPAPANDTVVVGIINFTEGIIVANMVAEMIEAHTDLKVVRKLNLGGVNVNFEAIKR |
| (SEQ | GGANNGIDIYVEYTGHGLVDILGFPATTDPEGAYETVKKEYRDKWNIVWLKPLGFNNTY |
| ID | TLTVKDELAKQYNLKTFSDLAKISDKLILGATMFFLEGPDGYPGLQKLYNFKFKHTKSM |
| NO: | DMGIRYTAIDNNEVQVIDAWATDGLLVSHKLKILEDDKAFFPPYYAAPIIRQDVLDKHP |
| 14) | ELKDVLNKLANQISLEEMQKLNYKVDGEGQDPAKVAKEFLKEKGLIPAPAPVSGWRLFK |
| | KISGGGGSAVGQDTQEVIVVPHSLPFKVVVISAILALVVLTIISLIILIMLWQKKPR |
| BLIN- | ATGGAGACAGACACACTCCTGCTATGGGTACTGCTGCTCTGGGTTCCAGGTTCCACTGG |
| Ach | TGACATGGTTTTCACCCTGGAGGACTTCGTTGGTGACTGGGAGCAGACCGCTGCTTACA |
| 4.12 | ACCTGGACCAGGTTCTGGAACAGGGTGGTGTGTCTTCTGTGCTCCAGACGCTGGCTGTT |
| DNA | TCTGTGACTCCGATCCAGCGTATCGTTCGTTCTGGTGAAAACGGTCTGAAAATCGACAT |
| (SEQ | CCACGTTATCATCCCGTACGAAGGTCTGTCTGCTGATCAGATGGCTCATATCGAAGAGG |
| ID | TCTTCAAAGTTGTTTACCCGGTTGACGACCACCACTTCAAAGTCATCATGGAGTACGGT |
| NO: | ACCCTGGTTATCGACGGTGTTACCCCGAACATGCTCAACTACTTCGGTCGTCCGTACGA |
| 15) | GGGTATCGCTGTTTTCGACGGTAAGAAGATCACCGTTACCGGTACCCTGTGGAACGGTA |
| | ACAAAATCATCGACGAACGTCTGATCACCCCGGACGGTTCTATGCTGTTCCGTGTTACG |
| | ATCAACCCGGCACCAGCGCCGGCAAATGACACAGTCGTTGTCGGAATCATTAATTTCAC |
| | GGAAGGGATAATCGTTGCCAATATGGTTGCTGAGATGATCGAAGCGCACACCGACCTTA |
| | AAGTTGTACGCAAGTTGAACTTGGGCGGAGTAAATGTTAATTTTGAGGCGATAAAACGA |
| | GGCGGAGCCAATAACGGTATAGACATATATGTAGAGTATACTGGACATGGGCTGGTCGA |
| | TATTCTCGGGTTCCCTGCAACGACTGATCCAGAAGGTGCTTATGAAACTGTTAAAAAGG |
| | AATATAGGGATAAATGGAATATCGTGTGGCTCAAGCCACTCGGTTTCAATAATACCTAC |
| | ACTCTTACCGTCAAGGATGAGTTGGCGAAACAATATAATCTTAAAACGTTCTCAGACTT |
| | GGCGAAAATAAGTGACAAGCTGATCCTCGGAGCTACGATGTTCTTTTTGGAGGGTCCAG |
| | ACGGCTACCCCGGATTGCAAAAGTTGTATAACTTTAAATTTAAGCATACCAAGTCTATG |
| | GATATGGGCATCCGCTACACCGCCATTGACAACAACGAAGTGCAGGTTATTGACGCTTG |
| | GGCTACGGACGGGCTCCTCGTTAGCCACAAATTGAAGATCCTGGAAGATGATAAGGCTT |
| | TTTTTCCACCGTATTATGCAGCGCCAATAATAAGACAAGATGTGCTGGATAAGCATCCG |
| | GAGCTTAAGGACGTTTTGAATAAGCTTGCCAACCAAATTAGTCTGGAAGAAATGCAAAA |
| | ATTGAATTATAAGGTGGATGGTGAAGGTCAGGACCCAGCTAAGGTCGCCAAGGAGTTCC |
| | TGAAGGAGAAGGGACTGATCCCTGCTCCAGCACCAGTGTCCGGCTGGCGGCTGTTCAAG |
| | AAAATTTCTGGAGGCGGTGGCAGCGCTGTGGGCCAGGACACGCAGGAGGTCATCGTGGT |
| | GCCACACTCCTTGCCCTTTAAGGTGGTGGTGATCTCAGCCATCCTGGCCCTGGTGGTGC |
| | TCACCATCATCTCCCTTATCATCCTCATCATGCTTTGGCAGAAGAAGCCACGTTAG |
| BLIN- | METDTLLLWVLLLWVPGSTGDMVFTLEDFVGDWEQTAAYNLDQVLEQGGVSSVLQTLAV |
| Ach | SVTPIQRIVRSGENGLKIDIHVIIPYEGLSADQMAHIEEVFKVVYPVDDHHFKVIMEYG |
| 4.12 | TLVIDGVTPNMLNYFGRPYEGIAVFDGKKITVTGTLWNGNKIIDERLITPDGSMLFRVT |
| AA | INPAPAPANDTVVVGIINFTEGIIVANMVAEMIEAHTDLKVVRKLNLGGVNVNFEAIKR |
| (SEQ | GGANNGIDIYVEYTGHGLVDILGFPATTDPEGAYETVKKEYRDKWNIVWLKPLGFNNTY |
| ID | TLTVKDELAKQYNLKTFSDLAKISDKLILGATMFFLEGPDGYPGLQKLYNFKFKHTKSM |
| NO: | DMGIRYTAIDNNEVQVIDAWATDGLLVSHKLKILEDDKAFFPPYYAAPIIRQDVLDKHP |
| 16) | ELKDVLNKLANQISLEEMQKLNYKVDGEGQDPAKVAKEFLKEKGLIPAPAPVSGWRLFK |
| | KISGGGGSAVGQDTQEVIVVPHSLPFKVVVISAILALVVLTIISLIILIMLWQKKPR* |
| BLIN- | ATGGAGACAGACACACTCCTGCTATGGGTACTGCTGCTCTGGGTTCCAGGTTCCACTGG |
| GABA | TGACATGGTTTTCACCCTGGAGGACTTCGTTGGTGACTGGGAGCAGACCGCTGCTTACA |
| 2.10 | ACCTGGACCAGGTTCTGGAACAGGGTGGTGTGTCTTCTGTGCTCCAGACGCTGGCTGTT |
| DNA | TCTGTGACTCCGATCCAGCGTATCGTTCGTTCTGGTGAAAACGGTCTGAAAATCGACAT |
| (SEQ | CCACGTTATCATCCCGTACGAAGGTCTGTCTGCTGATCAGATGGCTCATATCGAAGAGG |
| ID | TCTTCAAAGTTGTTTACCCGGTTGACGACCACCACTTCAAAGTCATCATGGAGTACGGT |
| NO: | ACCCTGGTTATCGACGGTGTTACCCCGAACATGCTCAACTACTTCGGTCGTCCGTACGA |
| 17) | GGGTATCGCTGTTTTCGACGGTAAGAAGATCACCGTTACCGGTACCCTGTGGAACGGTA |
| | ACAAAATCATCGACGAACGTCTGATCACCCCGGACGGTTCTATGCTGTTCCGTGTTACG |
| | ATCAACCCGGCACCAGCGCCGTCTGAGAGTATCAATTTCGTCTCTTGGGGAGGATCTAC |
| | ACAAGATGCACAGAAGCAGGCCTGGGCGGATCCGTTTTCTAAGGCCTCAGGCATAACAG |
| | TCGTTCAAGACGGCCCTACCGATTATGGTAAGCTTAAAGCGATGGTCGAGTCCGGCAAT |
| | GTACAATGGGATGTCGTAGACGTAGAGGCTGATTTCGCACTGAGAGCGGCGGCAGAGGG |
| | TCTTTTGGAGCCGTTGGATTTCAGTGTGATACAACGGGACAAGATCGACCCTCGATTTG |
| | TCAGCGACCACGGGGTCGGTTCCTTCTTTTTTTCTTTTGTACTGGGTTACAATGAGGGC |
| | AAACTCGGAGCGTCCAAGCCTCAGGATTGGACGGCTCTTTTTGACACGAAAACCTATCC |
| | AGGGAAACGAGCCCTCTACAAGTGGCCGAGCCCGGGGGTACTTGAGCTTGCACTCCTGG |
| | CTGACGGGGTACCAGCCGACAAATTGTATCCTCTGGACCTGGATCGGGCGTTTAAGAAA |
| | CTTGATACGATCAAGAAAGACATTGTGTGGTGGGGAGGGGGTGCTCAAAGTCAACAGCT |
| | GCTGGCGAGTGGGGAAGTGTCAATGGGCCAGTTCTGGAACGGGAGGATTCACGCTTTGC |
| | AAGAAGATGGAGCACCTGTGGGGGTCTCATGGAAGCAGAACCTCGTCATGGCCGACATA |
| | CTGGTGGTTCCTAAAGGGACCAAGAATAAAGCGGCTGCAATGAAGTTCCTCGCTAGCGC |
| | TTCTTCTGCTAAAGGACAAGACGATTTTTCCAACCTCACCGCATACGCGCCCGTCAATA |
| | TAGACTCAGTACAACGCCTCGATAGTGTACTGGCGCCAAATTTGCCCACTGCATATGTG |
| | AAAGATCAGATTACACTGGATTTCGCGTATTGGGCCAAGAATGGCCCCGCAATAGCAAC |
| | GCGCTGGAACGAGTGGCTCGTAAAGTTGCAGGTTGACCCTGCTCCAGCACCAGTGTCCG |
| | GCTGGCGGCTGTTCAAGAAAATTTCTGGAGGCGGTGGCAGCGCTGTGGGCCAGGACACG |
| | CAGGAGGTCATCGTGGTGCCACACTCCTTGCCCTTTAAGGTGGTGGTGATCTCAGCCAT |
| | CCTGGCCCTGGTGGTGCTCACCATCATCTCCCTTATCATCCTCATCATGCTTTGGCAGA |
| | AGAAGCCACGT |
| BLIN- | METDTLLLWVLLLWVPGSTGDMVFTLEDFVGDWEQTAAYNLDQVLEQGGVSSVLQTLAV |
| GABA | SVTPIQRIVRSGENGLKIDIHVIIPYEGLSADQMAHIEEVFKVVYPVDDHHFKVIMEYG |
| 2.10 | TLVIDGVTPNMLNYFGRPYEGIAVFDGKKITVTGTLWNGNKIIDERLITPDGSMLFRVT |
| AA | INPAPAPSESINFVSWGGSTQDAQKQAWADPFSKASGITVVQDGPTDYGKLKAMVESGN |
| (SEQ | VQWDVVDVEADFALRAAAEGLLEPLDFSVIQRDKIDPRFVSDHGVGSFFFSFVLGYNEG |
| ID | KLGASKPQDWTALFDTKTYPGKRALYKWPSPGVLELALLADGVPADKLYPLDLDRAFKK |
| NO: | LDTIKKDIVWWGGGAQSQQLLASGEVSMGQFWNGRIHALQEDGAPVGVSWKQNLVMADI |
| 18) | LVVPKGTKNKAAAMKFLASASSAKGQDDFSNLTAYAPVNIDSVQRLDSVLAPNLPTAYV |
| | KDQITLDFAYWAKNGPAIATRWNEWLVKLQVDPAPAPVSGWRLFKKISGGGGSAVGQDT |
| | QEVIVVPHSLPFKVVVISAILALVVLTIISLIILIMLWQKKPR |
| BLIN- | ATGGAGACAGACACACTCCTGCTATGGGTACTGCTGCTCTGGGTTCCAGGTTCCACTGG |
| GABA | TGACATGGTTTTCACCCTGGAGGACTTCGTTGGTGACTGGGAGCAGACCGCTGCTTACA |
| 4.10 | ACCTGGACCAGGTTCTGGAACAGGGTGGTGTGTCTTCTGTGCTCCAGACGCTGGCTGTT |
| DNA | TCTGTGACTCCGATCCAGCGTATCGTTCGTTCTGGTGAAAACGGTCTGAAAATCGACAT |
| (SEQ | CCACGTTATCATCCCGTACGAAGGTCTGTCTGCTGATCAGATGGCTCATATCGAAGAGG |
| ID | TCTTCAAAGTTGTTTACCCGGTTGACGACCACCACTTCAAAGTCATCATGGAGTACGGT |
| NO: | ACCCTGGTTATCGACGGTGTTACCCCGAACATGCTCAACTACTTCGGTCGTCCGTACGA |
| 19) | GGGTATCGCTGTTTTCGACGGTAAGAAGATCACCGTTACCGGTACCCTGTGGAACGGTA |
| | ACAAAATCATCGACGAACGTCTGATCACCCCGGACGGTTCTATGCTGTTCCGTGTTACG |
| | ATCAACCCGGCACCAGCGCCGTCTGAGAGTATCAATTTCGTCTCTTGGGGAGGATCTAC |
| | ACAAGATGCACAGAAGCAGGCCTGGGCGGATCCGTTTTCTAAGGCCTCAGGCATAACAG |
| | TCGTTCAAGACGGCCCTACCGATTATGGTAAGCTTAAAGCGATGGTCGAGTCCGGCAAT |
| | GTACAATGGGATGTCGTAGACGTAGAGGCTGATTTCGCACTGAGAGCGGCGGCAGAGGG |
| | TCTTTTGGAGCCGTTGGATTTCAGTGTGATACAACGGGACAAGATCGACCCTCGATTTG |
| | TCAGCGACCACGGGGTCGGTTCCTTCTTGTTTTCTTTTGTACTGGGTTACAATGAGGGC |
| | AAACTCGGAGCGTCCAAGCCTCAGGATTGGACGGCTCTTTTTGACACGAAAACCTATCC |
| | AGGGAAACGAGCCCTCTACAAGTGGCCGAGCCCGGGGGTACTTGAGCTTGCACTCCTGG |
| | CTGACGGGGTACCAGCCGACAAATTGTATCCTCTGGACCTGGATCGGGCGTTTAAGAAA |
| | CTTGATACGATCAAGAAAGACATTGTGTGGTGGGGAGGGGGTGCTCAAAGTCAACAGCT |
| | GCTGGCGAGTGGGGAAGTGTCAATGGGCCAGTTCTGGAACGGGAGGATTCACGCTTTGC |
| | AAGAAGATGGAGCACCTGTGGGGGTCTCATGGAAGCAGAACCTCGTCATGGCCGACATA |
| | CTGGTGGTTCCTAAAGGGACCAAGAATAAAGCGGCTGCAATGAAGTTCCTCGCTAGCGC |
| | TTCTTCTGCTAAAGGACAAGACGATTTTTCCGCACTCACCGCATACGCGCCCGTCAATA |
| | TAGACTCAGTACAACGCCTCGATGCTAAGCTGGCGCCAAATTTGCCCACTGCATATGTG |
| | AAAGATCAGATTACACTGGATTTCGCGTATTGGGCCAAGAATGGCCCCGCAATAGCAAC |
| | GCGCTGGAACGAGTGGCTCGTAAAGTTGCAGGTTGACCCTGCTCCAGCACCAGTGTCCG |
| | GCTGGCGGCTGTTCAAGAAAATTTCTGGAGGCGGTGGCAGCGCTGTGGGCCAGGACACG |
| | CAGGAGGTCATCGTGGTGCCACACTCCTTGCCCTTTAAGGTGGTGGTGATCTCAGCCAT |
| | CCTGGCCCTGGTGGTGCTCACCATCATCTCCCTTATCATCCTCATCATGCTTTGGCAGA |
| | AGAAGCCACGTTAGTAACATATGCTCTAGAGGGCCCGCGGTTCGAAGGTAAGCCTATCC |
| | CTAACCCTCTCCTCGGTCTCGATTCTACGCGTACCGGTCATCATCACCATCACCATTGA |
| BLIN- | METDTLLLWVLLLWVPGSTGDMVFTLEDFVGDWEQTAAYNLDQVLEQGGVSSVLQTLAV |
| GABA | SVTPIQRIVRSGENGLKIDIHVIIPYEGLSADQMAHIEEVEKVVYPVDDHHFKVIMEYG |
| 4.10 | TLVIDGVTPNMLNYFGRPYEGIAVFDGKKITVTGTLWNGNKIIDERLITPDGSMLFRVT |
| AA | INPAPAPSESINFVSWGGSTQDAQKQAWADPFSKASGITVVQDGPTDYGKLKAMVESGN |
| (SEQ | VQWDVVDVEADFALRAAAEGLLEPLDESVIQRDKIDPRFVSDHGVGSFLFSFVLGYNEG |
| ID | KLGASKPQDWTALFDTKTYPGKRALYKWPSPGVLELALLADGVPADKLYPLDLDRAFKK |
| NO: | LDTIKKDIVWWGGGAQSQQLLASGEVSMGQFWNGRIHALQEDGAPVGVSWKQNLVMADI |
| 20) | LVVPKGTKNKAAAMKFLASASSAKGQDDFSALTAYAPVNIDSVQRLDAKLAPNLPTAYV |
| | KDQITLDFAYWAKNGPAIATRWNEWLVKLQVDPAPAPVSGWRLFKKISGGGGSAVGQDT |
| | QEVIVVPHSLPFKVVVISAILALVVLTIISLIILIMLWQKKPR**HML*RARGSKVSLS |
| | LTLSSVSILRVPVIITITI |
| BLIN- | gacggatcgggagatctcccgatcccctatggtcgactctcagtacaatctgctctgat |
| GABA | gccgcatagttaagccagtatctgctccctgcttgtgtgttggaggtcgctgagtagtg |
| 4.11 | cgcgagcaaaatttaagctacaacaaggcaaggcttgaccgacaattgcatgaagaatc |
| DNA | tgcttagggttaggcgttttgcgctgcttcgcgatgtacgggccagatatacgcgttga |
| (SEQ | cattgattattgactagttattaatagtaatcaattacggggtcattagttcatagccc |
| ID | atatatggagttccgcgttacataacttacggtaaatggcccgcctggctgaccgccca |
| NO: | acgacccccgcccattgacgtcaataatgacgtatgttcccatagtaacgccaataggg |
| 21) | actttccattgacgtcaatgggggactatttacggtaaactgcccacttggcagtaca |
| | tcaagtgtatcatatgccaagtacgccccctattgacgtcaatgacggtaaatggcccg |
| | cctggcattatgcccagtacatgaccttatgggactttcctacttggcagtacatctac |
| | gtattagtcatcgctattaccatggtgatgcggttttggcagtacatcaatgggcgtgg |
| | atagcggtttgactcacggggatttccaagtctccaccccattgacgtcaatgggagtt |
| | tgttttggcaccaaaatcaacgggactttccaaaatgtcgtaacaactccgccccattg |
| | acgcaaatgggcggtaggcgtgtacggtgggaggtctatataagcagagctctctggct |
| | aactagagaacccactgcttactggcttatcgaaattaatacgactcactatagggaga |
| | cccaagctggctagttAAGCTTGCCatggagacagacacactcctgctatgggtactgc |
| | tgctctgggttccaggttccactggtgacatggttttcaccctggaagacttcgttggt |
| | gactgggagcagaccgctgcttacaacctggaccaggttctggaacagggtggtgtgtc |
| | ttctgtgctccagacgctggctgtttctgtgactccgatccagcgtatcgttcgttctg |
| | gtgaaaacggtctgaaaatcgacatccacgttatcatcccgtacgaaggtctgtctgct |
| | gatcagatggctcatatcgaagaggtcttcaaagttgtttacccggttgacgaccacca |
| | cttcaaagtcatcatggagtacggtaccctggttatcgacggtgttaccccgaacatgc |
| | tcaactacttcggtcgtccgtacgagggtatcgctgttttcgacggtaagaagatcacc |
| | gttaccggtaccctgtggaacggtaacaaaatcatcgacgaacgtctgatcaccccgga |
| | cggttctatgctgttccgtgttacgatcaacggaggaggaggcGGTTCTGAGAGTATCA |
| | ATTTCGTCTCTTGGGGAGGATCTACACAAGATGCACAGAAGCAGGCCTGGGCGGATCCG |
| | TTTTCTAAGGCCTCAGGCATAACAGTCGTTCAAGACGGCCCTACCGATTATGGTAAGCT |
| | TAAAGCGATGGTCGAGTCCGGCAATGTACAATGGGATGTCGTAGACGTAGAGGCTGATT |
| | TCGCACTGAGAGCGGCGGCAGAGGGTCTTTTGGAGCCGTTGGATTTCAGTGTGATACAA |
| | CGGGACAAGATCGACCCTCGATTTGTCAGCGACCACGGGGTCGGTTCCTTCTTgTTTTC |
| | TTTTGTACTGGGTTACAATGAGGGCAAACTCGGAGCGTCCAAGCCTCAGGATTGGACGG |
| | CTCTTTTTGACACGAAAACCTATCCAGGGAAACGAGCCCTCTACAAGTGGCCGAGCCCG |
| | GGGGTACTTGAGCTTGCACTCCTGGCTGACGGGGTACCAGCCGACAAATTGTATCCTCT |
| | GGACCTGGATCGGGCGTTTAAGAAACTTGATACGATCAAGAAAGACATTGTGTGGTGGG |
| | GAGGGGGTGCTCAAAGTCAACAGCTGCTGGCGAGTGGGGAAGTGTCAATGGGCCAGTTC |
| | TGGAACGGGAGGATTCACGCTTTGCAAGAAGATGGAGCACCTGTGGGGGTCTCATGGAA |
| | GCAGAACCTCGTCATGGCCGACATACTGGTGGTTCCTAAAGGGACCAAGAATAAAGCGG |
| | CTGCAATGAAGTTCCTCGCTAGCGCTTCTTCTGCTAAAGGACAAGACGATTTTTCCgca |
| | CTCACCGCATACGCGCCCGTCAATATAGACTCAGTACAACGCCTCGATgctaagCTGGC |
| | GCCAAATTTGCCCACTGCATATGTGAAAGATCAGATTACACTGGATTTCGCGTATTGGG |
| | CCAAGAATGGCCCCGCAATAGCAACGCGCTGGAACGAGTGGCTCGTAAAGTTGCAGGTT |
| | GACCCTGCTCCAGCACCAGTGTCCggctggcggctgttcAAGAAAattTCTGGAGGCGG |
| | TGGCAGCgctgtgggccaggacacgcaggaggtcatcgtggtgccacactccttgccct |
| | ttaaggtggtggtgatctcagccatcctggccctggtggtgctcaccatcatctccctt |
| | atcatcctcatcatgctttggcagaagaagccacgttagTAAcatatgcTCTAGAgggc |
| | ccgcggttcgaaggtaagcctatccctaaccctctcctcggtctcgattctacgcgtac |
| | cggtcatcatcaccatcaccattgagtttaaacccgctgatcagcctcgactgtgcctt |
| | ctagttgccagccatctgttgtttgcccctcccccgtgccttccttgaccctggaaggt |
| | gccactcccactgtcctttcctaataaaatgaggaaattgcatcgcattgtctgagtag |
| | gtgtcattctattctggggggtggggtggggcaggacagcaagggggaggattgggaag |
| | acaatagcaggcatgctggggatgcggtgggctctatggcttctgaggcggaaagaacc |
| | agctggggctctagggggtatccccacgcgccctgtagcggcgcattaagcgcggcggg |
| | tgtggtggttacgcgcagcgtgaccgctacacttgccagcgccctagcgcccgctcctt |
| | tcgctttcttcccttcctttctcgccacgttcgccggctttccccgtcaagctctaaat |
| | cggggcatccctttagggttccgatttagtgctttacggcacctcgaccccaaaaaact |
| | tgattagggtgatggttcacgtagtgggccatcgccctgatagacggtttttcgccctt |
| | tgacgttggagtccacgttctttaatagtggactcttgttccaaactggaacaacactc |
| | aaccctatctcggtctattcttttgatttataagggattttggggatttcggcctattg |
| | gttaaaaaatgagctgatttaacaaaaatttaacgcgaattaattctgtggaatgtgtg |
| | tcagttagggtgtggaaagtccccaggctccccaggcaggcagaagtatgcaaagcatg |
| | catctcaattagtcagcaaccaggtgtggaaagtccccaggctccccagcaggcagaag |
| | tatgcaaagcatgcatctcaattagtcagcaaccatagtcccgcccctaactccgccca |
| | tcccgcccctaactccgcccagttccgcccattctccgccccatggctgactaattttt |
| | tttatttatgcagaggccgaggccgcctctgcctctgagctattccagaagtagtgagg |
| | aggcttttttggaggcctaggcttttgcaaaaagctcccgggagcttgtatatccattt |
| | tcggatctgatcaagagacaggatgaggatcgtttcgcatgattgaacaagatggattg |
| | cacgcaggttctccggccgcttgggtggagaggctattcggctatgactgggcacaaca |
| | gacaatcggctgctctgatgccgccgtgttccggctgtcagcgcaggggcgcccggttc |
| | tttttgtcaagaccgacctgtccggtgccctgaatgaactgcaggacgaggcagcgcgg |
| | ctatcgtggctggccacgacgggcgttccttgcgcagctgtgctcgacgttgtcactga |
| | agcgggaagggactggctgctattgggcgaagtgccggggcaggatctcctgtcatctc |
| | accttgctcctgccgagaaagtatccatcatggctgatgcaatgcggcggctgcatacg |
| | cttgatccggctacctgcccattcgaccaccaagcgaaacatcgcatcgagcgagcacg |
| | tactcggatggaagccggtcttgtcgatcaggatgatctggacgaagagcatcaggggc |
| | tcgcgccagccgaactgttcgccaggctcaaggcgcgcatgcccgacggcgaggatctc |
| | gtcgtgacccatggcgatgcctgcttgccgaatatcatggtggaaaatggccgcttttc |
| | tggattcatcgactgtggccggctgggtgtggcggaccgctatcaggacatagcgttgg |
| | ctacccgtgatattgctgaagagcttggcggcgaatgggctgaccgcttcctcgtgctt |
| | tacggtatcgccgctcccgattcgcagcgcatcgccttctatcgccttcttgacgagtt |
| | cttctgagcgggactctggggttcgcgaaatgaccgaccaagcgacgcccaacctgcca |
| | tcacgagatttcgattccaccgccgccttctatgaaaggttgggcttcggaatcgtttt |
| | ccgggacgccggctggatgatcctccagcgcggggatctcatgctggagttcttcgccc |
| | accccaacttgtttattgcagcttataatggttacaaataaagcaatagcatcacaaat |
| | ttcacaaataaagcatttttttcactgcattctagttgtggtttgtccaaactcatcaa |
| | tgtatcttatcatgtctgtataccgtcgacctctagctagagcttggcgtaatcatggt |
| | catagctgtttcctgtgtgaaattgttatccgctcacaattccacacaacatacgagcc |
| | ggaagcataaagtgtaaagcctggggtgcctaatgagtgagctaactcacattaattgc |
| | gttgcgctcactgcccgctttccagtcgggaaacctgtcgtgccagctgcattaatgaa |
| | tcggccaacgcgcggggagaggcggtttgcgtattgggcgctcttccgcttcctcgctc |
| | actgactcgctgcgctcggtcgttcggctgcggcgagcggtatcagctcactcaaaggc |
| | ggtaatacggttatccacagaatcaggggataacgcaggaaagaacatgtgagcaaaag |
| | gccagcaaaaggccaggaaccgtaaaaaggccgcgttgctggcgtttttccataggctc |
| | cgcccccctgacgagcatcacaaaaatcgacgctcaagtcagaggtggcgaaacccgac |
| | aggactataaagataccaggcgtttccccctggaagctccctcgtgcgctctcctgttc |
| | cgaccctgccgcttaccggatacctgtccgcctttctcccttcgggaagcgtggcgctt |
| | tctcaatgctcacgctgtaggtatctcagttcggtgtaggtcgttcgctccaagctggg |
| | ctgtgtgcacgaaccccccgttcagcccgaccgctgcgccttatccggtaactatcgtc |
| | ttgagtccaacccggtaagacacgacttatcgccactggcagcagccactggtaacagg |
| | attagcagagcgaggtatgtaggcggtgctacagagttcttgaagtggtggcctaacta |
| | cggctacactagaaggacagtatttggtatctgcgctctgctgaagccagttaccttcg |
| | gaaaaagagttggtagctcttgatccggcaaacaaaccaccgctggtagcggtggtttt |
| | tttgtttgcaagcagcagattacgcgcagaaaaaaaggatctcaagaagatcctttgat |
| | cttttctacggggtctgacgctcagtggaacgaaaactcacgttaagggattttggtca |
| | tgagattatcaaaaaggatcttcacctagatccttttaaattaaaaatgaagttttaaa |
| | tcaatctaaagtatatatgagtaaacttggtctgacagttaccaatgcttaatcagtga |
| | ggcacctatctcagcgatctgtctatttcgttcatccatagttgcctgactccccgtcg |
| | tgtagataactacgatacgggagggcttaccatctggccccagtgctgcaatgataccg |
| | cgagacccacgctcaccggctccagatttatcagcaataaaccagccagccggaagggc |
| | cgagcgcagaagtggtcctgcaactttatccgcctccatccagtctattaattgttgcc |
| | gggaagctagagtaagtagttcgccagttaatagtttgcgcaacgttgttgccattgct |
| | acaggcatcgtggtgtcacgctcgtcgtttggtatggcttcattcagctccggttccca |
| | acgatcaaggcgagttacatgatcccccatgttgtgcaaaaaagcggttagctccttcg |
| | gtcctccgatcgttgtcagaagtaagttggccgcagtgttatcactcatggttatggca |
| | gcactgcataattctcttactgtcatgccatccgtaagatgcttttctgtgactggtga |
| | gtactcaaccaagtcattctgagaatagtgtatgcggcgaccgagttgctcttgcccgg |
| | cgtcaatacgggataataccgcgccacatagcagaactttaaaagtgctcatcattgga |
| | aaacgttcttcggggcgaaaactctcaaggatcttaccgctgttgagatccagttcgat |
| | gtaacccactcgtgcacccaactgatcttcagcatcttttactttcaccagcgtttctg |
| | ggtgagcaaaaacaggaaggcaaaatgccgcaaaaaagggaataagggcgacacggaaa |
| | tgttgaatactcatactcttcctttttcaatattattgaagcatttatcagggttattg |
| | tctcatgagcggatacatatttgaatgtatttagaaaaataaacaaataggggttccgc |
| | gcacatttccccgaaaagtgccacctgacgtc |
| BLIN- | METDTLLLWVLLLWVPGSTGDMVFTLEDFVGDWEQTAAYNLDQVLEQGGVSSVLQTLAV |
| GABA | SVTPIQRIVRSGENGLKIDIHVIIPYEGLSADQMAHIEEVFKVVYPVDDHHFKVIMEYG |
| 4.11 | TLVIDGVTPNMLNYFGRPYEGIAVFDGKKITVTGTLWNGNKIIDERLITPDGSMLFRVT |
| AA | INGGGGGSESINFVSWGGSTQDAQKQAWADPFSKASGITVVQDGPTDYGKLKAMVESGN |
| (SEQ | VQWDVVDVEADFALRAAAEGLLEPLDFSVIQRDKIDPRFVSDHGVGSFLFSFVLGYNEG |
| ID | KLGASKPQDWTALFDTKTYPGKRALYKWPSPGVLELALLADGVPADKLYPLDLDRAFKK |
| NO: | LDTIKKDIVWWGGGAQSQQLLASGEVSMGQFWNGRIHALQEDGAPVGVSWKQNLVMADI |
| 22 | LVVPKGTKNKAAAMKFLASASSAKGQDDFSALTAYAPVNIDSVQRLDAKLAPNLPTAYV |
| | KDQITLDFAYWAKNGPAIATRWNEWLVKLQVDPAPAPVSGWRLFKKISGGGGSAVGQDT |
| | QEVIVVPHSLPFKVVVISAILALVVLTIISLIILIMLWQKKPR |

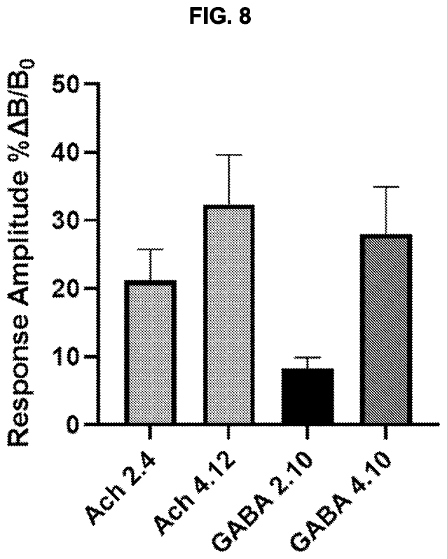

FIG. 8 shows the measured bioluminescence response amplitudes for the BLIN-Ach 2.4 and BLIN-Ach 4.12 Ach sensor variants and the BLIN-GABA 2.10 and BLIN-GABA 4.10 GABA sensor variants in the presence of their respective neurotransmitters.

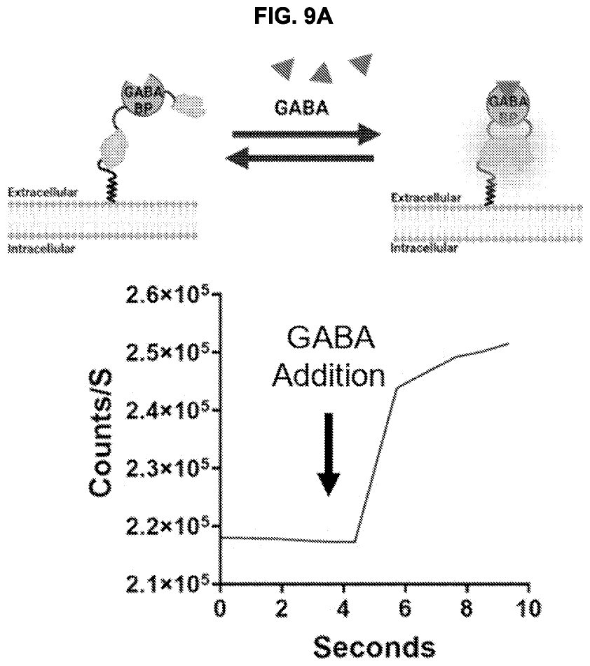

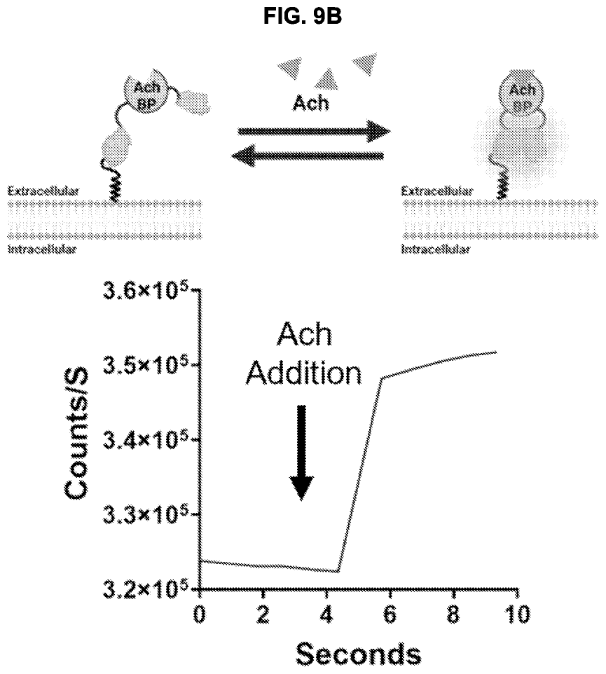

FIG. 9A shows the design and luminescent response for the top performing GABA sensor variant, BLIN-GABA 4.11, when GABA neurotransmitter was added, and luminescence counts were recorded with a plate reader over time. FIG. 9B shows the design and luminescent response for the top performing Ach sensor variant, BLIN-Ach 4.10, when Ach neurotransmitter was added, and luminescence counts were recorded with a plate reader over time.

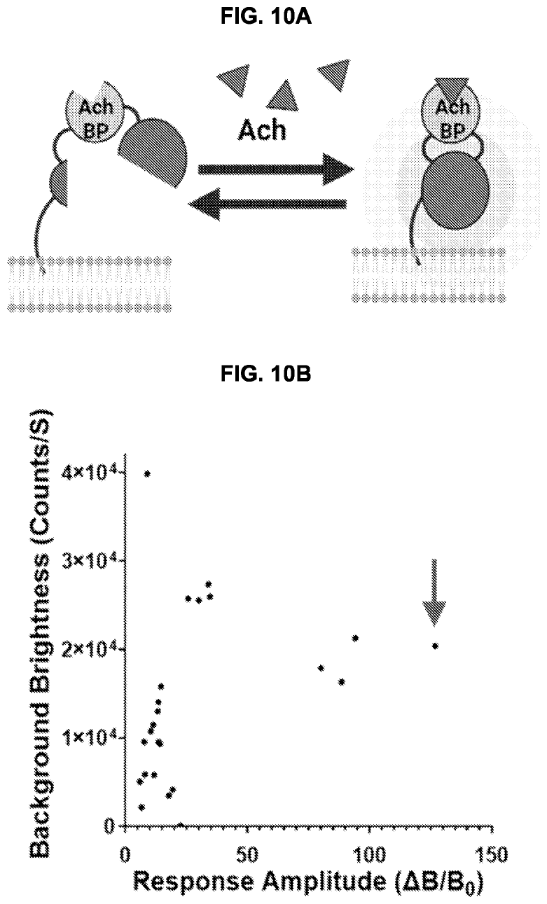

FIG. 10A-B show the design and luminescent response of exemplary Ach sensors. FIG. 10A shows the design of exemplary BLIN-Ach sensors. FIG. 10B shows the luminescent response vs. background luminescence (i.e., noise) of an Ach sensor library. The arrow indicates the top performing Ach sensor variant from the library, BLIN-Ach 4.10, that exhibited the best signal to noise ratio.

### Example 6

### Bioluminescent Serotonin Sensors

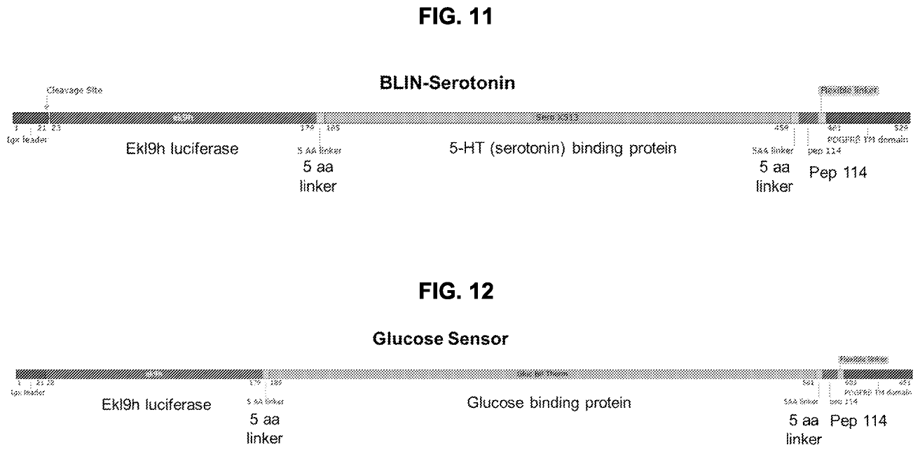

Bioluminescent sensor variants that luminesce in the presence of the neurotransmitter serotonin have also been prepared (BLIN-Serotonin) (FIG. 11). These exemplary BLIN-Serotonin sensor variants utilize an N-terminal Ek19H luciferase (shown in Table 2) or a NanoLuc small bit (smBit) luciferase, and comprise a 5 amino acid linker, a 5-HT (serotonin) binding protein, another 5 amino acid linker, and Pep 114 (FIG. 11). The 5-HT (serotonin) binding protein is described by Unger et al., “Directed evolution of a selective and sensitive serotonin sensor via machine learning,” Cell 183 (7): 1986-2002 (2020), the entire contents of which are fully incorporated herein by reference. This exemplary BLIN-Serotonin neurotransmitter sensor variant was created using the same methodologies as described in Example 1 for the BLING constructs. The DNA and amino acid sequences for the BLIN-Serotonin construct are shown in Table 5.

TABLE 5
BLIN (Serotonin) Variant Polynucleotide and
Amino Acid Sequences

| | |
|---|---|
| BLIN- | atggagacagacacactcctgctatgggtactgctgct |
| Sero- | ctgggttccaggttccactggtgacatggttttcaccc |
| tonin | tggaagacttcgttggtgactgggagcagaccgctgct |
| DNA | tacaacctggaccaggttctggaacagggtggtgtgtc |
| (SEQ | ttctgtgctccagacgctggctgtttctgtgactccga |
| ID | tccagcgtatcgttcgttctggtgaaaacggtctgaaa |
| NO: | atcgacatccacgttatcatcccgtacgaaggtctgtc |
| 23) | tgctgatcagatggctcatatcgaagaggtcttcaaag |
| | ttgtttacccggttgacgaccaccacttcaaagtcatc |
| | atggagtacggtaccctggttatcgacggtgttacccc |
| | gaacatgctcaactacttcggtcgtccgtacgagggta |
| | tcgctgttttcgacggtaagaagatcaccgttaccggt |
| | accctgtggaacggtaacaaaatcatcgacgaacgtct |
| | gatcaccccggacggttctatgctgttccgtgttacga |
| | tcaacggaggaggaggcGGTGCGAACGACACCGTCGTT |
| | GTGGGCTCCATCAACCATACAGAGCAGATCATCGTCGC |
| | AAACATGTTGGCAGAGATGATAGAGGCGCATACAGACC |
| | TTAAGGTGGTTCGCAAGCTGAACCTAGGCGGGGTTAAT |
| | GTTAACTTTGAAGCCATCAAACGAGGAGGAGCCAATAA |
| | TGGAATTGACATTTACCTGGAGTACGTCGGGTATGGTC |
| | TTGTGGATATACTGGGGATGGAATTTGCTACCGACCCA |
| | GAAGGTGCATACGAAACAGTGAAGAAGGAGTACAAACG |
| | GAAATGGAATATTGTATGGCTCAAACCACTGGGATTCA |
| | ACGCTTCCTATGTGTTGGCGGTGAAGGACGAACTGGCC |
| | AAACAGTATAACCTTAAGACGTTTAGTGACTTAGCAAA |
| | GATCAGCGACAAGCTGATACTGGGCGCAAACATGATGT |
| | TTCTCGAAAATCCCGATGGGTACCCTGGCCTGCAAAAA |
| | CTCTACAATTTCAAGTTCAAGCACACCAAATCTATGGA |
| | CGCTGGTATCCCTTATACCGCCATTGATAATAACGAAG |
| | TGCAGGTCATCGATGCCACTGCCACTGATGGCTTGCTC |
| | GTGAGCCACAAACTTAAGATTCTGGAGGATGATAAAGC |
| | CTTCTTCCCGCCATATTATGCTGCCCCCATCATCAGAC |
| | AGGATGTCTTAGATAAGCACCCTGAGCTGAAGGACGTG |
| | CTGAACAAACTCGCTAATCAAATTTCACTGGAAGAGAT |
| | GCAGAAACTAAATTACAAGAGGGACGGCGAGGGGCAGG |
| | ACCCCGCCAAAGTAGCTAAGGAGTTTTTGAAGGAGAAG |
| | GGACTCATTGGATCCggaggcggcGTGTCCggctggcg |
| | gctgttcAAGAAAattTCTGGAGGCGGTGGCAGCgctg |
| | tgggccaggacacgcaggaggtcatcgtggtgccacac |
| | tccttgccctttaaggtggtggtgatctcagccatcct |
| | ggccctggtggtgctcaccatcatctcccttatcatcc |
| | tcatcatgctttggcagaagaagccacgt |
| BLIN- | METDTLLLWVLLLWVPGSTGDMVFTLEDFVGDWEQTAA |
| Sero- | YNLDQVLEQGGVSSVLQTLAVSVTPIQRIVRSGENGLK |
| tonin | IDIHVIIPYEGLSADQMAHIEEVEKVVYPVDDHHFKVI |
| AA | MEYGTLVIDGVTPNMLNYFGRPYEGIAVFDGKKITVTG |
| (SEQ | TLWNGNKIIDERLITPDGSMLFRVTINGGGGGANDTVV |
| ID | VGSINHTEQIIVANMLAEMIEAHTDLKVVRKLNLGGVN |
| NO: | VNFEAIKRGGANNGIDIYLEYVGYGLVDILGMEFATDP |
| 24) | EGAYETVKKEYKRKWNIVWLKPLGFNASYVLAVKDELA |
| | KQYNLKTFSDLAKISDKLILGANMMFLENPDGYPGLQK |
| | LYNFKFKHTKSMDAGIPYTAIDNNEVQVIDATATDGLL |
| | VSHKLKILEDDKAFFPPYYAAPIIRQDVLDKHPELKDV |
| | LNKLANQISLEEMQKLNYKRDGEGQDPAKVAKEFLKEK |
| | GLIGSGGGVSGWRLFKKISGGGGSAVGQDTQEVIVVPH |
| | SLPFKVVVISAILALVVLTIISLIILIMLWQKKPR |

### Example 7

### Bioluminescent Glucose Sensors

Bioluminescent sensor variants that luminesce in the presence of glucose have also been prepared (FIG. 12). These exemplary glucose sensor variants utilize an N-terminal Ek19H luciferase (shown in Table 2) or a NanoLuc small bit (smBit) luciferase, and comprise a 5 amino acid linker, a glucose binding protein, another 5 amino acid linker, and Pep 114 (FIG. 12). This glucose sensor variant was created using the same methodologies as described in Example 1 for the BLING constructs. The DNA and amino acid sequences for the glucose sensor construct are shown in Table 6.

TABLE 6
Glucose Sensor Variant Polynucleotide and
Amino Acid Sequences

| | |
|---|---|
| Glucose | atggagacagacacactcctgctatgggtactg |
| Sensor | ctgctctgggttccaggttccactggtgacatg |
| DNA | gttttcaccctggaagacttcgttggtgactgg |
| (SEQ | gagcagaccgctgcttacaacctggaccaggtt |
| ID | ctggaacagggtggtgtgtcttctgtgctccag |
| NO: | acgctggctgtttctgtgactccgatccagcgt |
| 25) | atcgttcgttctggtgaaaacggtctgaaaatc |
| | gacatccacgttatcatcccgtacgaaggtctg |
| | tctgctgatcagatggctcatatcgaagaggtc |
| | ttcaaagttgtttacccggttgacgaccaccac |
| | ttcaaagtcatcatggagtacggtaccctggtt |
| | atcgacggtgttaccccgaacatgctcaactac |
| | ttcggtcgtccgtacgagggtatcgctgttttc |
| | gacggtaagaagatcaccgttaccggtaccctg |
| | tggaacggtaacaaaatcatcgacgaacgtctg |
| | atcaccccggacggttctatgctgttccgtgtt |
| | acgatcaacggaggaggaggcGGTGGGGATAGG |
| | TCAAAATTGGAAATATTCTCATGGTGGGCGGGG |
| | GACGAAGGTCCTGCGTTGGAAGCACTCATCCGC |
| | CTCTATAAGCAAAAGTACCCCGGAGTGGAGGTG |
| | ATCAATGCGACGGTCACAGGGGGCGCAGGAGTG |
| | AACGCCCGCGCAGTTCTCAAAACGAGAATGCTG |
| | GGCGGAGATCCACCAGACACATTCCAGGTTCAC |
| | GCTGGGATGGAGCTGATCGGGACGTGGGTCGTC |
| | GCCAACAGGATGGAGGATCTTAGTGCTCTCTTT |
| | CGACAGGAGGGTTGGTTGCAGGCGTTCCCCAAG |
| | GGCTTGATCGACCTGATCTCCTACAAGGGCGGT |
| | ATATGGTCTGTCCCTGTAAATATCCACCGGAGT |
| | AATGTTATGTGGTACCTCCCCGCAAAGCTGAAG |
| | GAATGGGGGGTGAACCCACCGCGCACCTGGGAT |
| | GAGTTTCTGGCTACATGCCAGACCCTGAAACAA |
| | AAGGGCTTGGAGGCGCCGCTTGCGCTCGGTGAA |
| | AACTGGACACAACAGCATCTGTGGGAATCCGTT |
| | GCCCTCGCCGTACTGGGTCCCGACGACTGGAAT |
| | AATCTGTGGAATGGTAAACTTAAATTCACGGAT |
| | CCAAAGGCAGTACGCGCATGGGAAGTCTTCGGG |
| | CGGGTGCTGGACTGCGCGAATAAAGACGCAGCA |
| | GGTTTGAGCTGGCAACAAGCAGTTGATAGGGTA |
| | GTCCAAGGTAAGGCCGCTTTTAACGTCATGGGA |
| | GACTGGGCTGCTGGTTATATGACGACAACACTT |
| | AAACTCAAACCGGGCACGGACTTCGCGTGGGCT |
| | CCATCACCTGGCACTCAAGGAGTCTTCATGATG |
| | CTCTCTGACAGCTTCGGTCTGCCTAAAGGCGCT |
| | AAAAATCGGCAGAACGCCATTAATTGGTTGAGG |
| | CTTGTGGGGTCCAAAGAAGGACAGGATACATTT |
| | AATCCGCTCAAAGGCTCAATTGCCGCTAGGCTC |
| | GACAGCGACCCATCCAAATACAATGCCTACGGC |
| | CAGTCAGCCATGAGAGACTGGAGATCCAACCGA |
| | ATAGTAGGGAGTTTGGTGCATGGGGCTGTCGCG |
| | CCTGAGTCTTTTATGTCTCAATTCGGAACAGTT |
| | ATGGAAATATTCTTGCAAACCCGCAACCCCCAG |
| | GCCGCCGCTAACGCAGCCCAGGCAATTGCGGAT |
| | CAGGTCGGCCTTGGCAGGCTTGGACAGGGATCC |
| | ggaggcggcGTGTCCggctggcggctgttcAAG |
| | AAAattTCTGGAGGCGGTGGCAGCgctgtgggc |
| | caggacacgcaggaggtcatcgtggtgccacac |
| | tccttgccctttaaggtggtggtgatctcagcc |
| | atcctggccctggtggtgctcaccatcatctcc |
| | cttatcatcctcatcatgctttggcagaagaag |
| | ccacgt |
| Glucose | METDTLLLWVLLLWVPGSTGDMVFTLEDFVGDW |
| Sensor | EQTAAYNLDQVLEQGGVSSVLQTLAVSVTPIQR |
| AA | IVRSGENGLKIDIHVIIPYEGLSADQMAHIEEV |
| (SEQ | FKVVYPVDDHHFKVIMEYGTLVIDGVTPNMLNY |
| ID | FGRPYEGIAVFDGKKITVTGTLWNGNKIIDERL |
| NO: | ITPDGSMLFRVTINGGGGGGDRSKLEIFSWWAG |
| 26) | DEGPALEALIRLYKQKYPGVEVINATVTGGAGV |
| | NARAVLKTRMLGGDPPDTFQVHAGMELIGTWVV |
| | ANRMEDLSALFRQEGWLQAFPKGLIDLISYKGG |
| | IWSVPVNIHRSNVMWYLPAKLKEWGVNPPRTWD |
| | EFLATCQTLKQKGLEAPLALGENWTQQHLWESV |
| | ALAVLGPDDWNNLWNGKLKFTDPKAVRAWEVFG |
| | RVLDCANKDAAGLSWQQAVDRVVQGKAAFNVMG |
| | DWAAGYMTTTLKLKPGTDFAWAPSPGTQGVFMM |
| | LSDSFGLPKGAKNRQNAINWLRLVGSKEGQDTF |
| | NPLKGSIAARLDSDPSKYNAYGQSAMRDWRSNR |
| | IVGSLVHGAVAPESFMSQFGTVMEIFLQTRNPQ |
| | AAANAAQAIADQVGLGRLGQGSGGGVSGWRLFK |
| | KISGGGGSAVGQDTQEVIVVPHSLPFKVVVISA |
| | ILALVVLTIISLIILIMLWQKKPR |

1. A recombinant bioluminescent polypeptide sensor comprising:
(a) a luminescent signaling domain comprising a luciferase polypeptide that is split into two luciferase polypeptide domains;
(b) an analyte binding domain comprising one or more acetylcholine, GABA, serotonin, or glucose binding domains, or functional variants, mutants, or fragments thereof; and
(c) one or more peptide linkers;
wherein the analyte binding domain is present between the two luciferase polypeptide domains such that binding of an analyte to the analyte binding domain induces a conformational change in the luminescent signaling domain, thereby bringing the two luciferase polypeptide domains together to generate a luminescent signal.

2. The sensor of claim 1, further comprising (d) one or more cellular trafficking peptides comprising membrane trafficking peptides.

3. The sensor of claim 1, wherein the luminescent signaling domain is allosterically regulated by the analyte binding domain such that signaling from the luminescent signaling domain is altered upon interaction of the analyte binding domain with the analyte, and wherein signaling by the luminescent signaling domain is proportional to the level of interaction between the analyte binding domain and the analyte.

4-6. (canceled)

7. The sensor of claim 1, wherein the luciferase polypeptide emits luminescence at a wavelength ranging from about 450 nm to about 540 nm.

8-21. (canceled)

22. The sensor of claim 1, wherein the analyte binding domain binds specifically to the neurotransmitter acetylcholine, GABA, serotonin, or glucose.

23. The sensor of claim 1, wherein the sensor is encoded by a polynucleotide sequence having at least 90-99% identity to any one of SEQ ID NO: 11, 13, or 15, or wherein the sensor is encoded by a polynucleotide sequence selected from any one of SEQ ID NO: 11, 13, or 15.

24. (canceled)

25. The sensor of claim 1, wherein the sensor has an amino acid sequence having at least 90-99% identity to any one of SEQ ID NO: 12, 14, or 16, or wherein the sensor has an amino acid sequence selected from any one of SEQ ID NO:
12, 14, or 16.

26-28. (canceled)

29. The sensor of claim 1, wherein the sensor is encoded by a polynucleotide sequence having at least 90-99% identity to any one of SEQ ID NO: 17, 19, or 21, or wherein the sensor is encoded by a polynucleotide sequence selected from any one of SEQ ID NO: 17, 19, or 21.

30. (canceled)

31. The sensor of claim 1, wherein the sensor has an amino acid sequence having at least 90-99% identity to any one of SEQ ID NO: 18, 20, or 22, or wherein the sensor has an amino acid sequence selected from any one of SEQ ID NO:
18, 20, or 22.

32-34. (canceled)

35. The sensor of claim 1, wherein the sensor is encoded by a polynucleotide sequence having at least 90-99% identity to SEQ ID NO: 23, or wherein the sensor is encoded by a polynucleotide sequence selected from SEQ ID NO: 23.

36. (canceled)

37. The sensor of claim 1, wherein the sensor has an amino acid sequence having at least 90-99% identity to SEQ ID NO: 24, or wherein the sensor has an amino acid sequence selected from SEQ ID NO: 24.

38-40. (canceled)

41. The sensor of claim 1, wherein the sensor is encoded by a polynucleotide sequence having at least 90-99% identity to SEQ ID NO: 25, or wherein the sensor is encoded by a polynucleotide sequence selected from SEQ ID NO: 25.

42. (canceled)

43. The sensor of claim 1, wherein the sensor has an amino acid sequence having at least 90-99% identity to SEQ ID NO: 26, or wherein the sensor has an amino acid sequence selected from SEQ ID NO: 26.

44. (canceled)

45. A vector comprising a polynucleotide sequence encoding the recombinant bioluminescent polypeptide sensor of claim 1.

46. The vector of claim 45, wherein the vector is selected from a viral vector, a plasmid expression vector, an adeno-associated virus (AAV) vector, a recombinant AAV (rAAV) vector, a single-stranded AAV vector, a double-stranded AAV vector, or a self-complementary AAV (scAAV) vector.

47. The vector of claim 46, wherein the vector is an AAV vector of a serotype selected from AAV1, AAV2, AAV3, AAV4, AAV5, AAV6, AAV7, AAV8, AAV9, AAV10, AAV11, AAV12, or a hybrid serotype thereof, or wherein the vector is a pcDNA3.1 plasmid expression vector.

48. (canceled)

49. A cell comprising the vector of claim 45.

50. (canceled)

51. A method for detecting one or more analytes in a subject, the method comprising measuring a level of luminescence emitted by the recombinant bioluminescent polypeptide sensor of claim 1 and correlating the measured level of luminescence with the presence of the one or more analytes in the subject, wherein the recombinant bioluminescent polypeptide sensor is encoded and expressed from a polynucleotide sequence that is administered to the subject.

52. (canceled)

53. The method of claim 51, wherein the polynucleotide sequence has at least 90-99% identity to any one of SEQ ID NO: 11, 13, 15, 17, 19, 21, 23, or 25, or wherein the polynucleotide sequence is selected from any one of SEQ ID NO: 11, 13, 15, 17, 19, 21, 23, or 25.

54. (canceled)

55. The method of claim 51, wherein the recombinant bioluminescent polypeptide sensor has an amino acid sequence having at least 90-99% identity to any one of SEQ ID NO: 12, 14, 16, 18, 20, 22, 24, or 26, or wherein the recombinant bioluminescent polypeptide sensor has an amino acid sequence selected from any one of SEQ ID NO: 12, 14, 16, 18, 20, 22, 24, or 26.

56-60. (canceled)


SEQUENCE LISTING (26 sequences)

SEQ ID NO: 1 - DNA, 6991 bp, synthetic construct
  source 1..6991: mol_type = other DNA
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagttaagcttgccatgggagtcaaagttctgtttgccctgatctgcatcgctgtggccgaggccaagcccaccgagaacaacgaagacttcaacatcgtggccgtggccagcaacttcgcgaccacggatctcgatgctgaccgcgggaagttgcccggcaagaagctgccgctggaggtgctcaaagagctggaagccaatgcccggaaagctggctgcaccaggggctgtctgatctgcctgtcccacatcaagtgcacgcccaagatgaagaagttcatcccaggacgctgccacacctacgaaggcgacaaagagtccgcacagggaggaggcggtaccagtactctggacaaaatcgccaaaaacggtgtgattgtcgtcggtcaccgtgaatcttcagtgcctttctcttattacgacaatcagcaaaaagtggtgggttactcgcaggattactccaacgccattgttgaagcagtgaaaaagaaactcaacaaaccggacttgcaggtaaaactgattccgattacaacacaaaaccgtattccactgctgcaaaacggcactttcgattttgaatgtggttctaccaccaacaacgtcgaacgccaaaaacaggcggctttctctgacactattttcgtggtcggtacgcgcctgttgaccaaaaagggtggcgatatcaaagattttgccaacctgaaagacaaagccgtagtcgtcacttccggcactacctctgaagttttgctcaacaaactgaatgaagagcaaaaaatgaatatgcgcatcatcagcgccaaagatcacggtgactctttccgcaccctggaaagcggtcgtgccgttgcctttatgatggatgacgctctgctggccggtgaacgtgcgaaagcgaagaaaccagacaactgggaaatcgtcggcaagccgcagtctcaggaggcctacggttgtatgttgcgtaaagatgatccgcagttcaaaaagctgatggatgacaccatcgctcaggtgcagacctccggtgaagcggaaaaatggtttgataagtggttcaaaaatccaattccgccgaaaaacctgaacatgaatttcgaactgtcagacgaaatgaaagcactgttcaaagaaccgaatgacggatccggaggcggcggcggcataggcgaggcgatcgtcgacattcctgagattcctgggttcaaggacttggagcccctggagcagttcatcgcacaggtcgatctgtgtgtggactgcacaactggctgcctcaaagggcttgccaacgtgcagtgttctgacctgctcaagaagtggctgccgcaacgctgtgcgacctttgccagcaagatccagggccaggtggacaagatcaagggggccggtggtgacctcgaggctgtgggccaggacacgcaggaggtcatcgtggtgccacactccttgccctttaaggtggtggtgatctcagccatcctggccctggtggtgctcaccatcatctcccttatcatcctcatcatgctttggcagaagaagccacgttagtaacatatgctctagagggcccgcggttcgaaggtaagcctatccctaaccctctcctcggtctcgattctacgcgtaccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 2 - protein, 514 aa, synthetic construct
MGVKVLFALICIAVAEAKPTENNEDFNIVAVASNFATTDLDADRGKLPGKKLPLEVLKELEANARKAGCTRGCLICLSHIKCTPKMKKFIPGRCHTYEGDKESAQGGGGTSTLDKIAKNGVIVVGHRESSVPFSYYDNQQKVVGYSQDYSNAIVEAVKKKLNKPDLQVKLIPITTQNRIPLLQNGTFDFECGSTTNNVERQKQAAFSDTIFVVGTRLLTKKGGDIKDFANLKDKAVVVTSGTTSEVLLNKLNEEQKMNMRIISAKDHGDSFRTLESGRAVAFMMDDALLAGERAKAKKPDNWEIVGKPQSQEAYGCMLRKDDPQFKKLMDDTIAQVQTSGEAEKWFDKWFKNPIPPKNLNMNFELSDEMKALFKEPNDGSGGGGGIGEAIVDIPEIPGFKDLEPLEQFIAQVDLCVDCTTGCLKGLANVQCSDLLKKWLPQRCATFASKIQGQVDKIKGAGGDLEAVGQDTQEVIVVPHSLPFKVVVISAILALVVLTIISLIILIMLWQKKPR

SEQ ID NO: 3 - DNA, 7012 bp, synthetic construct
  source 1..7012: mol_type = other DNA
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagttaagcttgccatggagacagacacactcctgctatgggtactgctgctctgggttccaggttccactggtgacatggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtggaggaggcggtaccagtactctggacaaaatcgccaaaaacggtgtgattgtcgtcggtcaccgtgaatcttcagtgcctttctcttattacgacaatcagcaaaaagtggtgggttactcgcaggattactccaacgccattgttgaagcagtgaaaaagaaactcaacaaaccggacttgcaggtaaaactgattccgattacaacacaaaaccgtattccactgctgcaaaacggcactttcgattttgaatgtggttctaccaccaacaacgtcgaacgccaaaaacaggcggctttctctgacactattttcgtggtcggtacgcgcctgttgaccaaaaagggtggcgatatcaaagattttgccaacctgaaagacaaagccgtagtcgtcacttccggcactacctctgaagttttgctcaacaaactgaatgaagagcaaaaaatgaatatgcgcatcatcagcgccaaagatcacggtgactctttccgcaccctggaaagcggtcgtgccgttgcctttatgatggatgacgctctgctggccggtgaacgtgcgaaagcgaagaaaccagacaactgggaaatcgtcggcaagccgcagtctcaggaggcctacggttgtatgttgcgtaaagatgatccgcagttcaaaaagctgatggatgacaccatcgctcaggtgcagacctccggtgaagcggaaaaatggtttgataagtggttcaaaaatccaattccgccgaaaaacctgaacatgaatttcgaactgtcagacgaaatgaaagcactgttcaaagaaccgaatgacggatccggaggcggcctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgctcgaggctgtgggccaggacacgcaggaggtcatcgtggtgccacactccttgccctttaaggtggtggtgatctcagccatcctggccctggtggtgctcaccatcatctcccttatcatcctcatcatgctttggcagaagaagccacgttagtaacatatgctctagagggcccgcggttcgaaggtaagcctatccctaaccctctcctcggtctcgattctacgcgtaccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 4 - protein, 521 aa, synthetic construct
METDTLLLWVLLLWVPGSTGDMVFTLEDFVGDWRQTAGYNLDQVLEQGGVSSLFQNLGVSVTPIQRIVLSGENGLKIDIHVIIPYEGGGGGTSTLDKIAKNGVIVVGHRESSVPFSYYDNQQKVVGYSQDYSNAIVEAVKKKLNKPDLQVKLIPITTQNRIPLLQNGTFDFECGSTTNNVERQKQAAFSDTIFVVGTRLLTKKGGDIKDFANLKDKAVVVTSGTTSEVLLNKLNEEQKMNMRIISAKDHGDSFRTLESGRAVAFMMDDALLAGERAKAKKPDNWEIVGKPQSQEAYGCMLRKDDPQFKKLMDDTIAQVQTSGEAEKWFDKWFKNPIPPKNLNMNFELSDEMKALFKEPNDGSGGGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILALEAVGQDTQEVIVVPHSLPFKVVVISAILALVVLTIISLIILIMLWQKKPR

SEQ ID NO: 5 - DNA, 7009 bp, synthetic construct
  source 1..7009: mol_type = other DNA
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagttaagcttgccatggagacagacacactcctgctatgggtactgctgctctgggttccaggttccactggtgacatggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggaggaggaggcggtaccagtactctggacaaaatcgccaaaaacggtgtgattgtcgtcggtcaccgtgaatcttcagtgcctttctcttattacgacaatcagcaaaaagtggtgggttactcgcaggattactccaacgccattgttgaagcagtgaaaaagaaactcaacaaaccggacttgcaggtaaaactgattccgattacaacacaaaaccgtattccactgctgcaaaacggcactttcgattttgaatgtggttctaccaccaacaacgtcgaacgccaaaaacaggcggctttctctgacactattttcgtggtcggtacgcgcctgttgaccaaaaagggtggcgatatcaaagattttgccaacctgaaagacaaagccgtagtcgtcacttccggcactacctctgaagttttgctcaacaaactgaatgaagagcaaaaaatgaatatgcgcatcatcagcgccaaagatcacggtgactctttccgcaccctggaaagcggtcgtgccgttgcctttatgatggatgacgctctgctggccggtgaacgtgcgaaagcgaagaaaccagacaactgggaaatcgtcggcaagccgcagtctcaggaggcctacggttgtatgttgcgtaaagatgatccgcagttcaaaaagctgatggatgacaccatcgctcaggtgcagacctccggtgaagcggaaaaatggtttgataagtggttcaaaaatccaattccgccgaaaaacctgaacatgaatttcgaactgtcagacgaaatgaaagcactgttcaaagaaccgaatgacggatccggaggcggcgtgaccggctggcggctgtgcgaacgcattctgctcgaggctgtgggccaggacacgcaggaggtcatcgtggtgccacactccttgccctttaaggtggtggtgatctcagccatcctggccctggtggtgctcaccatcatctcccttatcatcctcatcatgctttggcagaagaagccacgttagtaacatatgctctagagggcccgcggttcgaaggtaagcctatccctaaccctctcctcggtctcgattctacgcgtaccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 6 - protein, 520 aa, synthetic construct
METDTLLLWVLLLWVPGSTGDMVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGGGGGTSTLDKIAKNGVIVVGHRESSVPFSYYDNQQKVVGYSQDYSNAIVEAVKKKLNKPDLQVKLIPITTQNRIPLLQNGTFDFECGSTTNNVERQKQAAFSDTIFVVGTRLLTKKGGDIKDFANLKDKAVVVTSGTTSEVLLNKLNEEQKMNMRIISAKDHGDSFRTLESGRAVAFMMDDALLAGERAKAKKPDNWEIVGKPQSQEAYGCMLRKDDPQFKKLMDDTIAQVQTSGEAEKWFDKWFKNPIPPKNLNMNFELSDEMKALFKEPNDGSGGGVTGWRLCERILLEAVGQDTQEVIVVPHSLPFKVVVISAILALVVLTIISLIILIMLWQKKPR

SEQ ID NO: 7 - DNA, 7003 bp, synthetic construct
  source 1..7003: mol_type = other DNA
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagttaagcttgccatggagacagacacactcctgctatgggtactgctgctctgggttccaggttccactggtgacatggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggttctccttctagtactctggacaaaatcgccaaaaacggtgtgattgtcgtcggtcaccgtgaatcttcagtgcctttctcttattacgacaatcagcaaaaagtggtgggttactcgcaggattactccaacgccattgttgaagcagtgaaaaagaaactcaacaaaccggacttgcaggtaaaactgattccgattacaacacaaaaccgtattccactgctgcaaaacggcactttcgattttgaatgtggttctaccaccaacaacgtcgaacgccaaaaacaggcggctttctctgacactattttcgtggtcggtacgcgcctgttgaccaaaaagggtggcgatatcaaagattttgccaacctgaaagacaaagccgtagtcgtcacttccggcactacctctgaagttttgctcaacaaactgaatgaagagcaaaaaatgaatatgcgcatcatcagcgccaaagatcacggtgactctttccgcaccctggaaagcggtcgtgccgttgcctttatgatggatgacgctctgctggccggtgaacgtgcgaaagcgaagaaaccagacaactgggaaatcgtcggcaagccgcagtctcaggaggcctacggttgtatgttgcgtaaagatgatccgcagttcaaaaagctgatggatgacaccatcgctcaggtgcagacctccggtgaagcggaaaaatggtttgataagtggttcaaaaatccaattccgccgaaaaacctgaacatgaatttcgaactgtcagacgaaatgaaagcactgttcaaagaaccgaatgaccctgctcctggcctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgctcgaggctgtgggccaggacacgcaggaggtcatcgtggtgccacactccttgccctttaaggtggtggtgatctcagccatcctggccctggtggtgctcaccatcatctcccttatcatcctcatcatgctttggcagaagaagccacgttagtaacatatgctctagagggcccgcggttcgaaggtaagcctatccctaaccctctcctcggtctcgattctacgcgtaccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 8 - protein, 518 aa, synthetic construct
METDTLLLWVLLLWVPGSTGDMVFTLEDFVGDWRQTAGYNLDQVLEQGGVSSLFQNLGVSVTPIQRIVLSGENGLKIDIHVIIPYEGSPSSTLDKIAKNGVIVVGHRESSVPFSYYDNQQKVVGYSQDYSNAIVEAVKKKLNKPDLQVKLIPITTQNRIPLLQNGTFDFECGSTTNNVERQKQAAFSDTIFVVGTRLLTKKGGDIKDFANLKDKAVVVTSGTTSEVLLNKLNEEQKMNMRIISAKDHGDSFRTLESGRAVAFMMDDALLAGERAKAKKPDNWEIVGKPQSQEAYGCMLRKDDPQFKKLMDDTIAQVQTSGEAEKWFDKWFKNPIPPKNLNMNFELSDEMKALFKEPNDPAPGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILALEAVGQDTQEVIVVPHSLPFKVVVISAILALVVLTIISLIILIMLWQKKPR

SEQ ID NO: 9 - DNA, 8100 bp, synthetic construct
  source 1..8100: mol_type = other DNA
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagttaagcttgccccatgaagacgatcatcgccctgagctacatcttctgcctggtgttcgccgactacaaggacgatgatgacgccatgaggactctgaacacctctgccatggacgggactgggctggtggtggagagggacttctctgttcgtatcctcactgcctgtttcctgtcgctgctcatcctgtccacgctcctggggaacacgctggtctgtgctgccgttatcaggttccgacacctgcggtccaaggtgaccaacttctttgtcatctccttggctgtgtcagatctcttggtggccgtcctggtcatgccctggaaggcagtggctgagattgctggcttctggccctttgggtccttctgtaacatctgggtggcctttgacatcatgtgctccactgcatccatcctcaacctctgtgtgatcagcgtggacaggtattgggctatctccagccctttccggtatgagagaaagatgacccccaaggcagccttcatcctgatcagtgtggcatggaccttgtctgtactcatctccttcatcccagtgcagctcagctggcacaaggcaaaacccacaagcccctctgatggaaatgccacttccctggctgagaccatagacaactgtgactccagcctcagcaggacatatgccatctcatcctctgtaatcagcttttacatccctgtggccatcatgattgtcacctacaccaggatctacaggattgctcagaaactgagctcactcattctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgggcggaggcggtagcgggggaggcggttctatggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtaatcatgaccaactgaaaagagaaactaaagtcctgaagactctgtcggtgatcatgggtgtgtttgtgtgctgttggctacctttcttcatcttgaactgcattttgcccttctgtgggtctggggagacgcagcccttctgcattgattccaacacctttgacgtgtttgtgtggtttgggtgggctaattcatccttgaaccccatcatttatgcctttaatgctgattttcggaaggcattttcaaccctcttaggatgctacagactttgccctgcgacgaataatgccatagagacggtgagtatcaataacaatggggccgcgatgttttccagccatcatgagccacgaggctccatctccaaggagtgcaatctggtttacctgatcccacatgctgtgggctcctctgaggacctgaaaaaggaggaggcagctggcatcgccagacccttggagaagctgtccccagccctatcggtcatattggactatgacactgacgtctctctggagaagatccaacccatcacacaaaacggtcagcacccaaccgggcccggaagcggagccactaacttctccctgttgaaacaagcaggggatgtcgaagagaatcccgggccagcggccgcacccgggccaatggtgagcaagggcgaggaggtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccactacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctgtacggcatggacgagctgtacaagtaacatatgctctagagggcccgcggttcgaaggtaagcctatccctaaccctctcctcggtctcgattctacgcgtaccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 10 - protein, 622 aa, synthetic construct
MKTIIALSYIFCLVFADYKDDDDAMRTLNTSAMDGTGLVVERDFSVRILTACFLSLLILSTLLGNTLVCAAVIRFRHLRSKVTNFFVISLAVSDLLVAVLVMPWKAVAEIAGFWPFGSFCNIWVAFDIMCSTASILNLCVISVDRYWAISSPFRYERKMTPKAAFILISVAWTLSVLISFIPVQLSWHKAKPTSPSDGNATSLAETIDNCDSSLSRTYAISSSVISFYIPVAIMIVTYTRIYRIAQKLSSLILSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILAGGGGSGGGGSMVFTLEDFVGDWRQTAGYNLDQVLEQGGVSSLFQNLGVSVTPIQRIVLSGENGLKIDIHVIIPYEGNHDQLKRETKVLKTLSVIMGVFVCCWLPFFILNCILPFCGSGETQPFCIDSNTFDVFVWFGWANSSLNPIIYAFNADFRKAFSTLLGCYRLCPATNNAIETVSINNNGAAMFSSHHEPRGSISKECNLVYLIPHAVGSSEDLKKEEAAGIARPLEKLSPALSVILDYDTDVSLEKIQPITQNGQHPTGP

SEQ ID NO: 11 - DNA, 1590 bp, synthetic construct
  source 1..1590: mol_type = other DNA
atggagacagacacactcctgctatgggtactgctgctctgggttccaggttccactggtgacatggttttcaccctggaagacttcgttggtgactgggagcagaccgctgcttacaacctggaccaggttctggaacagggtggtgtgtcttctgtgctccagacgctggctgtttctgtgactccgatccagcgtatcgttcgttctggtgaaaacggtctgaaaatcgacatccacgttatcatcccgtacgaaggtctgtctgctgatcagatggctcatatcgaagaggtcttcaaagttgtttacccggttgacgaccaccacttcaaagtcatcatggagtacggtaccctggttatcgacggtgttaccccgaacatgctcaactacttcggtcgtccgtacgagggtatcgctgttttcgacggtaagaagatcaccgttaccggtaccctgtggaacggtaacaaaatcatcgacgaacgtctgatcaccccggacggttctatgctgttccgtgttacgatcaacccggcaccagcgccggcaaatgacacagtcgttgtcggaagcaagaatttcacggaacaaataatcgttgccaatatggttgctgagatgatcgaagcgcacaccgaccttaaagttgtacgcaagttgaacttgggcggaacgaatgttaattttgaggcgataaaacgaggcggagccaataacggtatagacatatatgtagagtatactggaaccgggctggtcgatattctcgggatggaaccaacgactgatccagaaaaagcttatgaaactgttaaaaaggaatataaggataaatggaatatcgtgtggctcaagccactcggtttcaataatacctacactcttgctgtcaaggatgagttggcgaaacaatataatcttaaaacgttctcagacttggcgaaaataagtgacaagctgatcctcggagctacgatggagtttttggagcggccagacggctaccccggattgcaaaaggtatataactttaaatttaagcataccaagtctatggatatgggcatccgctacaccgccattgacaacaacgaagtgcaggttattgacgctttcgctacggacgggctcctcgttagccacaaattgaagatcctggaagatgataagcatttttttccaccgtattatgcagcgccaataataagacaagatgtgctggataagcatccggagcttaaggacgttttgaataagcttgccaaccaaattagtgatgaagaaatgcaaaaattgaattataaggtggatggtgaaggtcaggacccagctaaggtcgccaaggagttcctgaaggagaagggactgatcggatccggaggcggcgtgacgggatacagactgttcgaggagatccttggaggcggtggcagcgctgtgggccaggacacgcaggaggtcatcgtggtgccacactccttgccctttaaggtggtggtgatctcagccatcctggccctggtggtgctcaccatcatctcccttatcatcctcatcatgctttggcagaagaagccacgttag

SEQ ID NO: 12 - protein, 529 aa, synthetic construct
METDTLLLWVLLLWVPGSTGDMVFTLEDFVGDWEQTAAYNLDQVLEQGGVSSVLQTLAVSVTPIQRIVRSGENGLKIDIHVIIPYEGLSADQMAHIEEVFKVVYPVDDHHFKVIMEYGTLVIDGVTPNMLNYFGRPYEGIAVFDGKKITVTGTLWNGNKIIDERLITPDGSMLFRVTINPAPAPANDTVVVGSKNFTEQIIVANMVAEMIEAHTDLKVVRKLNLGGTNVNFEAIKRGGANNGIDIYVEYTGTGLVDILGMEPTTDPEKAYETVKKEYKDKWNIVWLKPLGFNNTYTLAVKDELAKQYNLKTFSDLAKISDKLILGATMEFLERPDGYPGLQKVYNFKFKHTKSMDMGIRYTAIDNNEVQVIDAFATDGLLVSHKLKILEDDKHFFPPYYAAPIIRQDVLDKHPELKDVLNKLANQISDEEMQKLNYKVDGEGQDPAKVAKEFLKEKGLIGSGGGVTGYRLFEEILGGGGSAVGQDTQEVIVVPHSLPFKVVVISAILALVVLTIISLIILIMLWQKKPR

SEQ ID NO: 13 - DNA, 7036 bp, synthetic construct
  source 1..7036: mol_type = other DNA
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagttaagcttgccatggagacagacacactcctgctatgggtactgctgctctgggttccaggttccactggtgacatggttttcaccctggaggacttcgttggtgactgggagcagaccgctgcttacaacctggaccaggttctggaacagggtggtgtgtcttctgtgctccagacgctggctgtttctgtgactccgatccagcgtatcgttcgttctggtgaaaacggtctgaaaatcgacatccacgttatcatcccgtacgaaggtctgtctgctgatcagatggctcatatcgaagaggtcttcaaagttgtttacccggttgacgaccaccacttcaaagtcatcatggagtacggtaccctggttatcgacggtgttaccccgaacatgctcaactacttcggtcgtccgtacgagggtatcgctgttttcgacggtaagaagatcaccgttaccggtaccctgtggaacggtaacaaaatcatcgacgaacgtctgatcaccccggacggttctatgctgttccgtgttacgatcaacccggcaccagcgccggcaaatgacacagtcgttgtcggaatcattaatttcacggaagggataatcgttgccaatatggttgctgagatgatcgaagcgcacaccgaccttaaagttgtacgcaagttgaacttgggcggagtaaatgttaattttgaggcgataaaacgaggcggagccaataacggtatagacatatatgtagagtatactggacatgggctggtcgatattctcgggttccctgcaacgactgatccagaaggtgcttatgaaactgttaaaaaggaatatagggataaatggaatatcgtgtggctcaagccactcggtttcaataatacctacactcttaccgtcaaggatgagttggcgaaacaatataatcttaaaacgttctcagacttggcgaaaataagtgacaagctgatcctcggagctacgatgttctttttggagggtccagacggctaccccggattgcaaaagttgtataactttaaatttaagcataccaagtctatggatatgggcatccgctacaccgccattgacaacaacgaagtgcaggttattgacgcttgggctacggacgggctcctcgttagccacaaattgaagatcctggaagatgataaggctttttttccaccgtattatgcagcgccaataataagacaagatgtgctggataagcatccggagcttaaggacgttttgaataagcttgccaaccaaattagtctggaagaaatgcaaaaattgaattataaggtggatggtgaaggtcaggacccagctaaggtcgccaaggagttcctgaaggagaagggactgatccctgctccagcaccagtgtccggctggcggctgttcaagaaaatttctggaggcggtggcagcgctgtgggccaggacacgcaggaggtcatcgtggtgccacactccttgccctttaaggtggtggtgatctcagccatcctggccctggtggtgctcaccatcatctcccttatcatcctcatcatgctttggcagaagaagccacgttagtaacatatgctctagagggcccgcggttcgaaggtaagcctatccctaaccctctcctcggtctcgattctacgcgtaccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 14 - protein, 529 aa, synthetic construct
METDTLLLWVLLLWVPGSTGDMVFTLEDFVGDWEQTAAYNLDQVLEQGGVSSVLQTLAVSVTPIQRIVRSGENGLKIDIHVIIPYEGLSADQMAHIEEVFKVVYPVDDHHFKVIMEYGTLVIDGVTPNMLNYFGRPYEGIAVFDGKKITVTGTLWNGNKIIDERLITPDGSMLFRVTINPAPAPANDTVVVGIINFTEGIIVANMVAEMIEAHTDLKVVRKLNLGGVNVNFEAIKRGGANNGIDIYVEYTGHGLVDILGFPATTDPEGAYETVKKEYRDKWNIVWLKPLGFNNTYTLTVKDELAKQYNLKTFSDLAKISDKLILGATMFFLEGPDGYPGLQKLYNFKFKHTKSMDMGIRYTAIDNNEVQVIDAWATDGLLVSHKLKILEDDKAFFPPYYAAPIIRQDVLDKHPELKDVLNKLANQISLEEMQKLNYKVDGEGQDPAKVAKEFLKEKGLIPAPAPVSGWRLFKKISGGGGSAVGQDTQEVIVVPHSLPFKVVVISAILALVVLTIISLIILIMLWQKKPR

SEQ ID NO: 15 - DNA, 1590 bp, synthetic construct
  source 1..1590: mol_type = other DNA
atggagacagacacactcctgctatgggtactgctgctctgggttccaggttccactggtgacatggttttcaccctggaggacttcgttggtgactgggagcagaccgctgcttacaacctggaccaggttctggaacagggtggtgtgtcttctgtgctccagacgctggctgtttctgtgactccgatccagcgtatcgttcgttctggtgaaaacggtctgaaaatcgacatccacgttatcatcccgtacgaaggtctgtctgctgatcagatggctcatatcgaagaggtcttcaaagttgtttacccggttgacgaccaccacttcaaagtcatcatggagtacggtaccctggttatcgacggtgttaccccgaacatgctcaactacttcggtcgtccgtacgagggtatcgctgttttcgacggtaagaagatcaccgttaccggtaccctgtggaacggtaacaaaatcatcgacgaacgtctgatcaccccggacggttctatgctgttccgtgttacgatcaacccggcaccagcgccggcaaatgacacagtcgttgtcggaatcattaatttcacggaagggataatcgttgccaatatggttgctgagatgatcgaagcgcacaccgaccttaaagttgtacgcaagttgaacttgggcggagtaaatgttaattttgaggcgataaaacgaggcggagccaataacggtatagacatatatgtagagtatactggacatgggctggtcgatattctcgggttccctgcaacgactgatccagaaggtgcttatgaaactgttaaaaaggaatatagggataaatggaatatcgtgtggctcaagccactcggtttcaataatacctacactcttaccgtcaaggatgagttggcgaaacaatataatcttaaaacgttctcagacttggcgaaaataagtgacaagctgatcctcggagctacgatgttctttttggagggtccagacggctaccccggattgcaaaagttgtataactttaaatttaagcataccaagtctatggatatgggcatccgctacaccgccattgacaacaacgaagtgcaggttattgacgcttgggctacggacgggctcctcgttagccacaaattgaagatcctggaagatgataaggctttttttccaccgtattatgcagcgccaataataagacaagatgtgctggataagcatccggagcttaaggacgttttgaataagcttgccaaccaaattagtctggaagaaatgcaaaaattgaattataaggtggatggtgaaggtcaggacccagctaaggtcgccaaggagttcctgaaggagaagggactgatccctgctccagcaccagtgtccggctggcggctgttcaagaaaatttctggaggcggtggcagcgctgtgggccaggacacgcaggaggtcatcgtggtgccacactccttgccctttaaggtggtggtgatctcagccatcctggccctggtggtgctcaccatcatctcccttatcatcctcatcatgctttggcagaagaagccacgttag

SEQ ID NO: 16 - protein, 529 aa, synthetic construct; identical to SEQ ID NO: 14

SEQ ID NO: 17 - DNA, 1722 bp, synthetic construct
  source 1..1722: mol_type = other DNA
atggagacagacacactcctgctatgggtactgctgctctgggttccaggttccactggtgacatggttttcaccctggaggacttcgttggtgactgggagcagaccgctgcttacaacctggaccaggttctggaacagggtggtgtgtcttctgtgctccagacgctggctgtttctgtgactccgatccagcgtatcgttcgttctggtgaaaacggtctgaaaatcgacatccacgttatcatcccgtacgaaggtctgtctgctgatcagatggctcatatcgaagaggtcttcaaagttgtttacccggttgacgaccaccacttcaaagtcatcatggagtacggtaccctggttatcgacggtgttaccccgaacatgctcaactacttcggtcgtccgtacgagggtatcgctgttttcgacggtaagaagatcaccgttaccggtaccctgtggaacggtaacaaaatcatcgacgaacgtctgatcaccccggacggttctatgctgttccgtgttacgatcaacccggcaccagcgccgtctgagagtatcaatttcgtctcttggggaggatctacacaagatgcacagaagcaggcctgggcggatccgttttctaaggcctcaggcataacagtcgttcaagacggccctaccgattatggtaagcttaaagcgatggtcgagtccggcaatgtacaatgggatgtcgtagacgtagaggctgatttcgcactgagagcggcggcagagggtcttttggagccgttggatttcagtgtgatacaacgggacaagatcgaccctcgatttgtcagcgaccacggggtcggttccttctttttttcttttgtactgggttacaatgagggcaaactcggagcgtccaagcctcaggattggacggctctttttgacacgaaaacctatccagggaaacgagccctctacaagtggccgagcccgggggtacttgagcttgcactcctggctgacggggtaccagccgacaaattgtatcctctggacctggatcgggcgtttaagaaacttgatacgatcaagaaagacattgtgtggtggggagggggtgctcaaagtcaacagctgctggcgagtggggaagtgtcaatgggccagttctggaacgggaggattcacgctttgcaagaagatggagcacctgtgggggtctcatggaagcagaacctcgtcatggccgacatactggtggttcctaaagggaccaagaataaagcggctgcaatgaagttcctcgctagcgcttcttctgctaaaggacaagacgatttttccaacctcaccgcatacgcgcccgtcaatatagactcagtacaacgcctcgatagtgtactggcgccaaatttgcccactgcatatgtgaaagatcagattacactggatttcgcgtattgggccaagaatggccccgcaatagcaacgcgctggaacgagtggctcgtaaagttgcaggttgaccctgctccagcaccagtgtccggctggcggctgttcaagaaaatttctggaggcggtggcagcgctgtgggccaggacacgcaggaggtcatcgtggtgccacactccttgccctttaaggtggtggtgatctcagccatcctggccctggtggtgctcaccatcatctcccttatcatcctcatcatgctttggcagaagaagccacgt

SEQ ID NO: 18 - protein, 574 aa, synthetic construct
METDTLLLWVLLLWVPGSTGDMVFTLEDFVGDWEQTAAYNLDQVLEQGGVSSVLQTLAVSVTPIQRIVRSGENGLKIDIHVIIPYEGLSADQMAHIEEVFKVVYPVDDHHFKVIMEYGTLVIDGVTPNMLNYFGRPYEGIAVFDGKKITVTGTLWNGNKIIDERLITPDGSMLFRVTINPAPAPSESINFVSWGGSTQDAQKQAWADPFSKASGITVVQDGPTDYGKLKAMVESGNVQWDVVDVEADFALRAAAEGLLEPLDFSVIQRDKIDPRFVSDHGVGSFFFSFVLGYNEGKLGASKPQDWTALFDTKTYPGKRALYKWPSPGVLELALLADGVPADKLYPLDLDRAFKKLDTIKKDIVWWGGGAQSQQLLASGEVSMGQFWNGRIHALQEDGAPVGVSWKQNLVMADILVVPKGTKNKAAAMKFLASASSAKGQDDFSNLTAYAPVNIDSVQRLDSVLAPNLPTAYVKDQITLDFAYWAKNGPAIATRWNEWLVKLQVDPAPAPVSGWRLFKKISGGGGSAVGQDTQEVIVVPHSLPFKVVVISAILALVVLTIISLIILIMLWQKKPR

SEQ ID NO: 19 - DNA, 1829 bp, synthetic construct
  source 1..1829: mol_type = other DNA
atggagacagacacactcctgctatgggtactgctgctctgggttccaggttccactggtgacatggttttcaccctggaggacttcgttggtgactgggagcagaccgctgcttacaacctggaccaggttctggaacagggtggtgtgtcttctgtgctccagacgctggctgtttctgtgactccgatccagcgtatcgttcgttctggtgaaaacggtctgaaaatcgacatccacgttatcatcccgtacgaaggtctgtctgctgatcagatggctcatatcgaagaggtcttcaaagttgtttacccggttgacgaccaccacttcaaagtcatcatggagtacggtaccctggttatcgacggtgttaccccgaacatgctcaactacttcggtcgtccgtacgagggtatcgctgttttcgacggtaagaagatcaccgttaccggtaccctgtggaacggtaacaaaatcatcgacgaacgtctgatcaccccggacggttctatgctgttccgtgttacgatcaacccggcaccagcgccgtctgagagtatcaatttcgtctcttggggaggatctacacaagatgcacagaagcaggcctgggcggatccgttttctaaggcctcaggcataacagtcgttcaagacggccctaccgattatggtaagcttaaagcgatggtcgagtccggcaatgtacaatgggatgtcgtagacgtagaggctgatttcgcactgagagcggcggcagagggtcttttggagccgttggatttcagtgtgatacaacgggacaagatcgaccctcgatttgtcagcgaccacggggtcggttccttcttgttttcttttgtactgggttacaatgagggcaaactcggagcgtccaagcctcaggattggacggctctttttgacacgaaaacctatccagggaaacgagccctctacaagtggccgagcccgggggtacttgagcttgcactcctggctgacggggtaccagccgacaaattgtatcctctggacctggatcgggcgtttaagaaacttgatacgatcaagaaagacattgtgtggtggggagggggtgctcaaagtcaacagctgctggcgagtggggaagtgtcaatgggccagttctggaacgggaggattcacgctttgcaagaagatggagcacctgtgggggtctcatggaagcagaacctcgtcatggccgacatactggtggttcctaaagggaccaagaataaagcggctgcaatgaagttcctcgctagcgcttcttctgctaaaggacaagacgatttttccgcactcaccgcatacgcgcccgtcaatatagactcagtacaacgcctcgatgctaagctggcgccaaatttgcccactgcatatgtgaaagatcagattacactggatttcgcgtattgggccaagaatggccccgcaatagcaacgcgctggaacgagtggctcgtaaagttgcaggttgaccctgctccagcaccagtgtccggctggcggctgttcaagaaaatttctggaggcggtggcagcgctgtgggccaggacacgcaggaggtcatcgtggtgccacactccttgccctttaaggtggtggtgatctcagccatcctggccctggtggtgctcaccatcatctcccttatcatcctcatcatgctttggcagaagaagccacgttagtaacatatgctctagagggcccgcggttcgaaggtaagcctatccctaaccctctcctcggtctcgattctacgcgtaccggtcatcatcaccatcaccattga

SEQ ID NO: 20 - protein, 606 aa, synthetic construct
METDTLLLWVLLLWVPGSTGDMVFTLEDFVGDWEQTAAYNLDQVLEQGGVSSVLQTLAVSVTPIQRIVRSGENGLKIDIHVIIPYEGLSADQMAHIEEVFKVVYPVDDHHFKVIMEYGTLVIDGVTPNMLNYFGRPYEGIAVFDGKKITVTGTLWNGNKIIDERLITPDGSMLFRVTINPAPAPSESINFVSWGGSTQDAQKQAWADPFSKASGITVVQDGPTDYGKLKAMVESGNVQWDVVDVEADFALRAAAEGLLEPLDFSVIQRDKIDPRFVSDHGVGSFLFSFVLGYNEGKLGASKPQDWTALFDTKTYPGKRALYKWPSPGVLELALLADGVPADKLYPLDLDRAFKKLDTIKKDIVWWGGGAQSQQLLASGEVSMGQFWNGRIHALQEDGAPVGVSWKQNLVMADILVVPKGTKNKAAAMKFLASASSAKGQDDFSALTAYAPVNIDSVQRLDAKLAPNLPTAYVKDQITLDFAYWAKNGPAIATRWNEWLVKLQVDPAPAPVSGWRLFKKISGGGGSAVGQDTQEVIVVPHSLPFKVVVISAILALVVLTIISLIILIMLWQKKPRHMLRARGSKVSLSLTLSSVSILRVPVIITITI

SEQ ID NO: 21 - DNA, 7171 bp, synthetic construct
  source 1..7171: mol_type = other DNA
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagttaagcttgccatggagacagacacactcctgctatgggtactgctgctctgggttccaggttccactggtgacatggttttcaccctggaagacttcgttggtgactgggagcagaccgctgcttacaacctggaccaggttctggaacagggtggtgtgtcttctgtgctccagacgctggctgtttctgtgactccgatccagcgtatcgttcgttctggtgaaaacggtctgaaaatcgacatccacgttatcatcccgtacgaaggtctgtctgctgatcagatggctcatatcgaagaggtcttcaaagttgtttacccggttgacgaccaccacttcaaagtcatcatggagtacggtaccctggttatcgacggtgttaccccgaacatgctcaactacttcggtcgtccgtacgagggtatcgctgttttcgacggtaagaagatcaccgttaccggtaccctgtggaacggtaacaaaatcatcgacgaacgtctgatcaccccggacggttctatgctgttccgtgttacgatcaacggaggaggaggcggttctgagagtatcaatttcgtctcttggggaggatctacacaagatgcacagaagcaggcctgggcggatccgttttctaaggcctcaggcataacagtcgttcaagacggccctaccgattatggtaagcttaaagcgatggtcgagtccggcaatgtacaatgggatgtcgtagacgtagaggctgatttcgcactgagagcggcggcagagggtcttttggagccgttggatttcagtgtgatacaacgggacaagatcgaccctcgatttgtcagcgaccacggggtcggttccttcttgttttcttttgtactgggttacaatgagggcaaactcggagcgtccaagcctcaggattggacggctctttttgacacgaaaacctatccagggaaacgagccctctacaagtggccgagcccgggggtacttgagcttgcactcctggctgacggggtaccagccgacaaattgtatcctctggacctggatcgggcgtttaagaaacttgatacgatcaagaaagacattgtgtggtggggagggggtgctcaaagtcaacagctgctggcgagtggggaagtgtcaatgggccagttctggaacgggaggattcacgctttgcaagaagatggagcacctgtgggggtctcatggaagcagaacctcgtcatggccgacatactggtggttcctaaagggaccaagaataaagcggctgcaatgaagttcctcgctagcgcttcttctgctaaaggacaagacgatttttccgcactcaccgcatacgcgcccgtcaatatagactcagtacaacgcctcgatgctaagctggcgccaaatttgcccactgcatatgtgaaagatcagattacactggatttcgcgtattgggccaagaatggccccgcaatagcaacgcgctggaacgagtggctcgtaaagttgcaggttgaccctgctccagcaccagtgtccggctggcggctgttcaagaaaatttctggaggcggtggcagcgctgtgggccaggacacgcaggaggtcatcgtggtgccacactccttgccctttaaggtggtggtgatctcagccatcctggccctggtggtgctcaccatcatctcccttatcatcctcatcatgctttggcagaagaagccacgttagtaacatatgctctagagggcccgcggttcgaaggtaagcctatccctaaccctctcctcggtctcgattctacgcgtaccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 22 - protein, 574 aa, synthetic construct
METDTLLLWVLLLWVPGSTGDMVFTLEDFVGDWEQTAAYNLDQVLEQGGVSSVLQTLAVSVTPIQRIVRSGENGLKIDIHVIIPYEGLSADQMAHIEEVFKVVYPVDDHHFKVIMEYGTLVIDGVTPNMLNYFGRPYEGIAVFDGKKITVTGTLWNGNKIIDERLITPDGSMLFRVTINGGGGGSESINFVSWGGSTQDAQKQAWADPFSKASGITVVQDGPTDYGKLKAMVESGNVQWDVVDVEADFALRAAAEGLLEPLDFSVIQRDKIDPRFVSDHGVGSFLFSFVLGYNEGKLGASKPQDWTALFDTKTYPGKRALYKWPSPGVLELALLADGVPADKLYPLDLDRAFKKLDTIKKDIVWWGGGAQSQQLLASGEVSMGQFWNGRIHALQEDGAPVGVSWKQNLVMADILVVPKGTKNKAAAMKFLASASSAKGQDDFSALTAYAPVNIDSVQRLDAKLAPNLPTAYVKDQITLDFAYWAKNGPAIATRWNEWLVKLQVDPAPAPVSGWRLFKKISGGGGSAVGQDTQEVIVVPHSLPFKVVVISAILALVVLTIISLIILIMLWQKKPR

SEQ ID NO: 23 - DNA, 1587 bp, synthetic construct
  source 1..1587: mol_type = other DNA
atggagacagacacactcctgctatgggtactgctgctctgggttccaggttccactggtgacatggttttcaccctggaagacttcgttggtgactgggagcagaccgctgcttacaacctggaccaggttctggaacagggtggtgtgtcttctgtgctccagacgctggctgtttctgtgactccgatccagcgtatcgttcgttctggtgaaaacggtctgaaaatcgacatccacgttatcatcccgtacgaaggtctgtctgctgatcagatggctcatatcgaagaggtcttcaaagttgtttacccggttgacgaccaccacttcaaagtcatcatggagtacggtaccctggttatcgacggtgttaccccgaacatgctcaactacttcggtcgtccgtacgagggtatcgctgttttcgacggtaagaagatcaccgttaccggtaccctgtggaacggtaacaaaatcatcgacgaacgtctgatcaccccggacggttctatgctgttccgtgttacgatcaacggaggaggaggcggtgcgaacgacaccgtcgttgtgggctccatcaaccatacagagcagatcatcgtcgcaaacatgttggcagagatgatagaggcgcatacagaccttaaggtggttcgcaagctgaacctaggcggggttaatgttaactttgaagccatcaaacgaggaggagccaataatggaattgacatttacctggagtacgtcgggtatggtcttgtggatatactggggatggaatttgctaccgacccagaaggtgcatacgaaacagtgaagaaggagtacaaacggaaatggaatattgtatggctcaaaccactgggattcaacgcttcctatgtgttggcggtgaaggacgaactggccaaacagtataaccttaagacgtttagtgacttagcaaagatcagcgacaagctgatactgggcgcaaacatgatgtttctcgaaaatcccgatgggtaccctggcctgcaaaaactctacaatttcaagttcaagcacaccaaatctatggacgctggtatcccttataccgccattgataataacgaagtgcaggtcatcgatgccactgccactgatggcttgctcgtgagccacaaacttaagattctggaggatgataaagccttcttcccgccatattatgctgcccccatcatcagacaggatgtcttagataagcaccctgagctgaaggacgtgctgaacaaactcgctaatcaaatttcactggaagagatgcagaaactaaattacaagagggacggcgaggggcaggaccccgccaaagtagctaaggagtttttgaaggagaagggactcattggatccggaggcggcgtgtccggctggcggctgttcaagaaaatttctggaggcggtggcagcgctgtgggccaggacacgcaggaggtcatcgtggtgccacactccttgccctttaaggtggtggtgatctcagccatcctggccctggtggtgctcaccatcatctcccttatcatcctcatcatgctttggcagaagaagccacgt

SEQ ID NO: 24 - protein, 529 aa, synthetic construct
METDTLLLWVLLLWVPGSTGDMVFTLEDFVGDWEQTAAYNLDQVLEQGGVSSVLQTLAVSVTPIQRIVRSGENGLKIDIHVIIPYEGLSADQMAHIEEVFKVVYPVDDHHFKVIMEYGTLVIDGVTPNMLNYFGRPYEGIAVFDGKKITVTGTLWNGNKIIDERLITPDGSMLFRVTINGGGGGANDTVVVGSINHTEQIIVANMLAEMIEAHTDLKVVRKLNLGGVNVNFEAIKRGGANNGIDIYLEYVGYGLVDILGMEFATDPEGAYETVKKEYKRKWNIVWLKPLGFNASYVLAVKDELAKQYNLKTFSDLAKISDKLILGANMMFLENPDGYPGLQKLYNFKFKHTKSMDAGIPYTAIDNNEVQVIDATATDGLLVSHKLKILEDDKAFFPPYYAAPIIRQDVLDKHPELKDVLNKLANQISLEEMQKLNYKRDGEGQDPAKVAKEFLKEKGLIGSGGGVSGWRLFKKISGGGGSAVGQDTQEVIVVPHSLPFKVVVISAILALVVLTIISLIILIMLWQKKPR

SEQ ID NO: 25 - DNA, 1953 bp, synthetic construct
  source 1..1953: mol_type = other DNA
atggagacagacacactcctgctatgggtactgctgctctgggttccaggttccactggtgacatggttttcaccctggaagacttcgttggtgactgggagcagaccgctgcttacaacctggaccaggttctggaacagggtggtgtgtcttctgtgctccagacgctggctgtttctgtgactccgatccagcgtatcgttcgttctggtgaaaacggtctgaaaatcgacatccacgttatcatcccgtacgaaggtctgtctgctgatcagatggctcatatcgaagaggtcttcaaagttgtttacccggttgacgaccaccacttcaaagtcatcatggagtacggtaccctggttatcgacggtgttaccccgaacatgctcaactacttcggtcgtccgtacgagggtatcgctgttttcgacggtaagaagatcaccgttaccggtaccctgtggaacggtaacaaaatcatcgacgaacgtctgatcaccccggacggttctatgctgttccgtgttacgatcaacggaggaggaggcggtggggataggtcaaaattggaaatattctcatggtgggcgggggacgaaggtcctgcgttggaagcactcatccgcctctataagcaaaagtaccccggagtggaggtgatcaatgcgacggtcacagggggcgcaggagtgaacgcccgcgcagttctcaaaacgagaatgctgggcggagatccaccagacacattccaggttcacgctgggatggagctgatcgggacgtgggtcgtcgccaacaggatggaggatcttagtgctctctttcgacaggagggttggttgcaggcgttccccaagggcttgatcgacctgatctcctacaagggcggtatatggtctgtccctgtaaatatccaccggagtaatgttatgtggtacctccccgcaaagctgaaggaatggggggtgaacccaccgcgcacctgggatgagtttctggctacatgccagaccctgaaacaaaagggcttggaggcgccgcttgcgctcggtgaaaactggacacaacagcatctgtgggaatccgttgccctcgccgtactgggtcccgacgactggaataatctgtggaatggtaaacttaaattcacggatccaaaggcagtacgcgcatgggaagtcttcgggcgggtgctggactgcgcgaataaagacgcagcaggtttgagctggcaacaagcagttgatagggtagtccaaggtaaggccgcttttaacgtcatgggagactgggctgctggttatatgacgacaacacttaaactcaaaccgggcacggacttcgcgtgggctccatcacctggcactcaaggagtcttcatgatgctctctgacagcttcggtctgcctaaaggcgctaaaaatcggcagaacgccattaattggttgaggcttgtggggtccaaagaaggacaggatacatttaatccgctcaaaggctcaattgccgctaggctcgacagcgacccatccaaatacaatgcctacggccagtcagccatgagagactggagatccaaccgaatagtagggagtttggtgcatggggctgtcgcgcctgagtcttttatgtctcaattcggaacagttatggaaatattcttgcaaacccgcaacccccaggccgccgctaacgcagcccaggcaattgcggatcaggtcggccttggcaggcttggacagggatccggaggcggcgtgtccggctggcggctgttcaagaaaatttctggaggcggtggcagcgctgtgggccaggacacgcaggaggtcatcgtggtgccacactccttgccctttaaggtggtggtgatctcagccatcctggccctggtggtgctcaccatcatctcccttatcatcctcatcatgctttggcagaagaagccacgt

SEQ ID NO: 26 - protein, 651 aa, synthetic construct
METDTLLLWVLLLWVPGSTGDMVFTLEDFVGDWEQTAAYNLDQVLEQGGVSSVLQTLAVSVTPIQRIVRSGENGLKIDIHVIIPYEGLSADQMAHIEEVFKVVYPVDDHHFKVIMEYGTLVIDGVTPNMLNYFGRPYEGIAVFDGKKITVTGTLWNGNKIIDERLITPDGSMLFRVTINGGGGGGDRSKLEIFSWWAGDEGPALEALIRLYKQKYPGVEVINATVTGGAGVNARAVLKTRMLGGDPPDTFQVHAGMELIGTWVVANRMEDLSALFRQEGWLQAFPKGLIDLISYKGGIWSVPVNIHRSNVMWYLPAKLKEWGVNPPRTWDEFLATCQTLKQKGLEAPLALGENWTQQHLWESVALAVLGPDDWNNLWNGKLKFTDPKAVRAWEVFGRVLDCANKDAAGLSWQQAVDRVVQGKAAFNVMGDWAAGYMTTTLKLKPGTDFAWAPSPGTQGVFMMLSDSFGLPKGAKNRQNAINWLRLVGSKEGQDTFNPLKGSIAARLDSDPSKYNAYGQSAMRDWRSNRIVGSLVHGAVAPESFMSQFGTVMEIFLQTRNPQAAANAAQAIADQVGLGRLGQGSGGGVSGWRLFKKISGGGGSAVGQDTQEVIVVPHSLPFKVVVISAILALVVLTIISLIILIMLWQKKPR